(19) United States
(12) Patent Application Publication
(10) Pub. No.: US 2009/0148843 A1
(43) Pub. Date: Jun. 11, 2009

(54) Means and Methods for the Prediction of Joint Destruction

(76) Inventors: Hendrik Schulze-Koops, Erlangen (DE); Alla Skapenko, Erlangen (DE); Peter E. Lipsky, Rockville, MD (US)

Correspondence Address: FULBRIGHT & JAWORSKI L.L.P., 600 CONGRESS AVE., SUITE 2400, AUSTIN, TX, 78701, US

(21) Appl. No.: 12/089,843

(22) PCT Filed: Oct. 12, 2006
(86) PCT No.: PCT/EP2006/009884

§ 371 Date: Nov. 25, 2008

(30) Foreign Application Priority Data
Oct. 13, 2005 (EP) 050223445

(51) Int. Cl. C12Q 1/68

(52) U.S. Cl. 435/6

(57) ABSTRACT

The present invention relates to a method of diagnosing and/or predicting joint destruction, early joint destruction and/or accelerated joint destruction in particular, in rheumatoid arthritis, comprising determining in a sample obtained from an individual the presence of at least one nucleic acid sequence encoding an IL-4 receptor (IL-4R) which contains a mutation in position 465 of the nucleotide sequence of the wild-type IL4R as shown in SEQ ID NO: 1, whereby at said position the nucleotide A is replaced or a nucleic acid sequence encoding an IL-4 receptor (IL-4R), said IL-4R comprising at position 75 as shown in SEQ ID NO: 2 a valine instead of an isoleucine. Furthermore, the invention provides for a method of diagnosing and/or predicting early joint destruction and/or accelerated joint destruction comprising determining in a sample obtained from an individual the presence of an encoded IL-4 receptor (IL-4R) which comprises at the homologous position 75 of IL-4 receptor as depicted in SEQ ID NO: 2 a mutation, said mutation comprising the exchange from an isoleucine to a valine. In addition, the present invention relates to a use of specific probes and/or primers for the preparation of a diagnostic composition for diagnosing and/or predicting early joint destruction and/or accelerated joint destruction in particular in rheumatoid arthritis.

The present invention relates to a method of diagnosing and/or predicting joint destruction, early joint destruction and/or accelerated joint destruction, in particular in rheumatoid arthritis, comprising determining in a sample obtained from an individual the presence of at least one nucleic acid sequence encoding an IL-4 receptor (IL-4R) which contains a mutation in position 465 of the nucleotide sequence of the wild-type IL4R as shown in SEQ ID NO: 1, whereby at said position the nucleotide A is replaced or a nucleic acid sequence encoding an IL-4 receptor (IL-4R), said IL-4R comprising at position 75 as shown in SEQ ID NO: 2 a valine instead of an isoleucine. Furthermore, the invention provides for a method of diagnosing and/or predicting early joint destruction and/or accelerated joint destruction comprising determining in a sample obtained from an individual the presence of an encoded IL-4 receptor (IL-4R) which comprises at the homologous position 75 of IL-4 receptor as depicted in SEQ ID NO: 2 a mutation, said mutation comprising the exchange from an isoleucine to a valine. In addition, the present invention relates to a use of specific probes and/or primers for the preparation of a diagnostic composition for diagnosing and/or predicting early joint destruction and/or accelerated joint destruction.

Rheumatoid arthritis (RA) is one of the most common human systemic autoimmune diseases characterized by synovial infiltrates and a progressive cell-mediated destruction of the joints, which results in substantial disability of the patients (Firestein (2003) Nature, 423(6937):356-361). Evidence from twin (Aho (1986) J Rheumatol, 13(5):899-902; Silman (1993) Br J Rheumatol, 32(10):903-907) and family (Deighton (1991) Ann Rheum Dis, 50(1):62-65) studies suggests that both genetic and environmental factors contribute to susceptibility to RA, and disease heritability has been estimated to be about 60% (MacGregor (2000) Arthritis Rheum, 43(1):30-37)). To date, only the human leukocyte antigen (HLA) locus on chromosome 6 has been consistently linked to and associated with RA susceptibility (Seldin (1999) Arthritis Rheum, 42(6):1071-1079). This association, however, accounts for only one-third of the genetic susceptibility (Deighton (1989) Clin Genet, 36(3):178-182). In an attempt to identify the non-HLA loci involved in disease susceptibility, genome-wide scans have been performed by different groups in studies involving RA multiplex families with affected siblings pairs (Cornelis (1998) Proc Natl Acad Sci USA, 95(18):10746-10750; Jawaheer (2001) Am J Hum Genet, 68(4):927-936; MacKay (2002) Arthritis Rheum, 46(3):632-639; Shiozawa (1998) Int Immunol, 10(12):1891-1895). Although no common loci apart from the HLA region were identified by all the studies, several non-HLA loci that demonstrate modest linkage with RA were suggested by multiple studies. One such locus is the chromosome 16p12, for which a modest association of 16p12 markers (D16S3103, D16S403, D16S420, and D16S401) with RA was observed in three out of the four studies.

The region located only about 2.5 Mb centromeric from D16S401 at 16p11.2-12.1 contains genes coding for specific receptor subunits of T helper type 2 (Th2) cytokines, interleukin 4 (IL4) and IL21. IL4 and IL4R play an important role in the pathogenesis of RA, as diminished production of IL4 was previously believed to contribute to the characteristic Th1-mediated autoimmune rheumatoid inflammation (Skapenko (1999) J Immunol, 163:491-499; Simon (1994) Proc Natl Acad Sci USA, 91:8562-8566). Within the coding region of the IL4R gene seven single nucleotide polymorphisms (SNPs) that result in amino acid substitutions have been identified (Deichmann (1997) Biochem Biophys Res Commun, 231(3):696-697; Kruse (1999) Immunology, 96(3):365-371; Hershey (1997) N Engl J Med 1997; 337(24):1720-1725). Two of them, the I50V (in the following also designated as “I75V”, in particular in relation to a polypeptide which also comprises a signal sequence) and the Q551R reside within sequences coding for functionally important regions of the IL4R molecule and were reported to be associated with functional alterations in cellular assays (Mitsuyasu (1998) Nat Genet, 19(2):119-120; Mitsuyasu (1999) J Immunol, 162(3):1227-1231; Hershey (1997) N Engl J Med, 337(24):1720-1725; Risma (2002) J Immunol, 169(3):1604-1610; Stephenson (2004) J Immunol, 173(7):4523-4528; Wang (1999) J Immunol, 162(8):4385-4389; Kruse (1999) Immunology, 96(3):365-371). In addition, the association of the IL4R gene with susceptibility to immune disorders mediated by an imbalance in the Th1/Th2 ratio has already been demonstrated, for example in asthma and atopy (Mitsuyasu (1998), loc. cit.; Kruse (1999) loc. cit.; Hershey (1997), loc. cit.), type 1 diabetes (Mirel (2002) Diabetes, 51(11):3336-3341; Bugawan (2003) Am J Hum Genet, 72(6):1505-1514) or lupus (Kanemitsu (1999) Arthritis Rheum, 42(6):1298-1300). However, even if IL4R was considered as a potential candidate for RA susceptibility, a concrete link to RA could not be provided and published data were contradicting.

The RA-characteristic joint destruction occurs in the majority of patients within the first two years of the disease (Lee (2001) Lancet, 358(9285):903-911). The demonstration that early suppression of inflammation may impede the progression of joint destruction even years after treatment (O'Dell (2002) Arthritis Rheum, 46(2):283-285) highlights the importance of identifying at-risk patients as early as possible. However, attempts to identify prognostic factors for a destructive vs. a non-destructive outcome yielded disappointing results. The presence of rheumatoid factor (RF) and, more recently, of antibodies to cyclic citrullinated peptides has been associated with more severe disease (De Rycke (2004) Ann Rheum Dis, 63(12):1587-1593), but, unfortunately, these factors are not reliable enough to allow therapeutic decision-making. The only genetic locus with a predictive value for the development of more severe disease identified to date is, also here, the HLA locus (Thomson (1999) Arthritis Rheum, 42(4):757-762; Gorman (2004) Arthritis Rheum 50(2):400-412). It was speculated that IL4R might be an alternative potential candidate for the prediction of disease progression, because the extent of the Th1 dominance during early disease, which is controlled at least in part by inhibitory signaling through IL4R, correlates with disease progression (Kanik (1998) J Rheumatol, 25(1):16-22; van der Graaff (1998) Lancet 1998; 351(9120):1931).

Thus, the technical problem underlying the present invention was to provide means and methods for an early determination of the potentially erosive and/or fast erosive character of bone and/or joint destruction, for example in rheumatoid arthritis. The solution to said technical problem is achieved by providing the embodiments as characterized in the claims and in the description.

In a first aspect, the present invention provides for a method of diagnosing and/or predicting joint destruction early joint destruction and/or accelerated joint destruction, in particular in rheumatoid arthritis, said method comprising determining in a sample obtained from an individual the presence of at least one nucleic acid sequence selected from the group consisting of
(a) a nucleic acid sequence encoding an IL-4 receptor (IL-4R) which contains a mutation in position 465 of the nucleotide sequence of the wild-type IL4R as shown in SEQ ID NO: 1 (cDNA sequence), whereby at said position the nucleotide A is replaced;
(b) a nucleic acid sequence encoding an IL-4 receptor (IL-4R) which contains a mutation in position 465 of the nucleotide sequence of the wild-type IL4R as shown in SEQ ID NO: 1, whereby at said position the nucleotide “A” (adenosine) is replaced by the nucleotide “G” (guanine);
(c) a nucleic acid sequence encoding an IL-4 receptor (IL-4R), said IL-4R comprising at position 75 as shown in SEQ ID NO: 2 a valine;
(d) a nucleic acid sequence encoding an IL-4 receptor (IL-4R), said IL-4R comprising at position 75 as shown in SEQ ID NO: 2 a valine instead of an isoleucine;
(e) a nucleic acid sequence comprising at least 15 nucleotides of the nucleic acid sequence of any one of (a) to (d) and comprising the mutation as defined in any one of (a) to (d);
(f) a nucleic aid sequence comprising a nucleotide sequence as shown in any one of SEQ ID NOS. 3 or a part thereof, said part comprising the nucleotide sequence “gtc” (or “guc” on the RNA level) as depicted in nucleotides numbers 5758 to 5760 of SEQ ID NO: 3;
(g) a nucleic acid sequence encoding a polypeptide comprising the amino acid sequence as shown in SEQ ID NO(s): 4;
(h) a nucleic acid sequence which hybridizes to a nucleotide sequence defined in any one of (a) to (g) and having a mutation as defined in any one of (a) to (d); and
(i) a nucleic acid sequence being degenerate as a result of the genetic code to the nucleic acid sequence as defined in (h).

The present invention relates, accordingly to an in vitro method for determining the susceptibility, predisposition, presence and/or potential risk of developing an erosive joint destruction, in a particular early after the onset of the disease (in particular rheumatoid arthrithis) in a subject/patient, said method comprising the steps of:
(a) obtaining a biological sample from said subject/patient;
(b) assaying said sample for the IL4R allel as defined herein, namely the herein defined IL4R single nucleotide polymorphism “I50V”/“I75V”; and
(c) comparing said allelic situation with the situation in a normal control (“I50I/I75I”) or a control showing the herein defined mutant single nucleotide polymorphism leading to an exchange of isoleucine (“I) to valine (“V”) on position 50 (or, when a signal peptide of 25 amino acids is concerned, position 75) of the expressed IL4R.

The herein defined point mutation/SNP or allelic situation of IL4R can be assessed on the level of the nucleic acid molecules (e.g. DNA or RNA) as well as on the protein level (i.e. expressed IL4R protein)

As detailed herein below and as also summarized in the experimental part of this invention the present invention provides for a method of predicting/diagnosing at an early point of time a modification in the joints and to predict in particular joint destruction, early joint destruction and/or accelerated joint destruction in particular in rheumatoid arthritis. This “early point of time” relates to a diagnosis and or prognosis even before signs of a joint and/or bone disorder, in particular joint or bone destruction and/or ersosion, can be detected clinically, radiographically, by MRI or by ultrasound. The gist of the present invention lays in the fact that a reliable marker is provided which allows the diagnosis of the further path or prognosis of joint destruction in particular in patients suffering (or being prone to suffer from) rheumatoid arthritis. The means and methods provided herein provide for the first time a reliable diagnostic marker with which it is possible to deduce whether a given individual/human patient is prone to erosive or non-erosive development and/or course of joint destruction, in particular in rheumatoid arthritis. The diagnostic marker provided herein is indicative for joint destruction, in particular, but not-limited to, “early (joint) destruction”. In context of this invention the term “early destruction” or “early joint destruction” does not only relate to the specific destruction of the joints but also to corresponding bone modifications.

Rheumatoid arthritis is diagnosed according to the 1987 revised criteria for the classification of rheumatoid arthritis (1. morning stiffness in an around joints lasting for at least one hour before maximal improvement present for at least six weeks; 2. arthritis of three or more joint areas observed by a physician present for at least six weeks; 3. arthritis of the proximal interphalangeal (PIP), metacarpophalangeal (MCP), or wrist joints present for at least six weeks; 4. symmetric arthritis present for at least six weeks; 5. subcutaneous nodules; 6. positive test for rheumatoid factor; 7. radiographic erosions or periarticular osteopenia in hand or wrist joints) if at least four of the following seven criteria are present: 1. morning stiffness in an around joints lasting for at least one hour before maximal improvement present for at least six weeks; 2. arthritis of three or more joint areas observed by a physician present for at least six weeks; 3. arthritis of the proximal interphalangeal (PIP), metacarpophalangeal (MCP), or wrist joints present for at least six weeks; 4. symmetric arthritis present for at least six weeks; 5. subcutaneous nodules; 6. positive test for rheumatoid factor; and 7. radiographic erosions or periarticular osteopenia in hand or wrist joints; see also Arnett (1988) Arthritis Rheum 31:315-324. Therefore, the person skilled in the art has means to diagnose the onset of RA by the above recited criteria.

An “early destruction” can be defined in context of this invention showing signs of joint and/or bone erosion before 5 years after first conventional diagnosis of RA, before 4 years after first conventional diagnosis of RA, before 3 years after first conventional diagnosis of RA and in particular before 2 years after first conventional diagnosis of RA. In this context, the “first conventional diagnosis of RA” relates to the determination of the onset of the disease. As documented in the appended examples, bone erosions occurred in more than 65% of the (mutant) V50 allele homozygous patients compared to about 37.0% of the patients homozygous for the (non-mutated) 150 allele at two years after disease onset (OR 3.86, p<0.0001). This association was independent of individual factors previously associated with severe disease, such as rheumatoid factor or the HLA-DR shared epitope. Also Machold (2006), Rheumatology (Advance Access published Aug. 9, 2006) teaches that erosive disease (“early erosion and/or early destruction”, developed in 63.6% of patients over 3 years with the majority (74.3%) appearing in the first and 97.2% by the end of the second year. Accordingly, the person skilled in the art considers “early destruction” as appear in less than 5, less than 4, less than 3 and in particular less than 2 years after the onset of rheumatoid arthritis. As shown in the appended examples, the herein identified mutant of IL4R has an impact on the rapidity of disease progression and the investigation was carried out on RA patients who have either erosive or non-erosive disease based on radiographic analysis on the hands and feet two years after disease onset. Therefore, the point mutation disclosed herein is a genetic marker with high predictive value for early erosive RA.

The methods, means and uses of this invention allow also the early determination of the potential risk of arthritis-related erosions which comprise, for example joint space narrowing and general erosion of articular bones or joint structures, normally within the first two years of disease. Accordingly, the methods and means provided herein allow for the early detection of rapid/rapidly erosive rheumatoid arthritis versus less or even non-erosive rheumatoid arthritis. However, it is to be understood that the means and methods provided herein in addition allow for the detection of the future fate, course or progress of erosive events in a patient suffering from rheumatoid arthritis, as will be detailed herein below. These evidences and erosions may, inter alia, be assessed by standardized radiographs, typically of hand and feet, ultrasound and/or by magnetic resonance imaging. In accordance with the teachings of the present invention, the attending physician may, at any point of time, choose a different treatment option, depending whether the patient, preferably the human patient to be treated, shows early signs of joint or bone destruction and is either homozygous for the here described “I50V”/“I75V” IL4R SNP (both alleles comprise the point mutation “V” on position 50/75 as defined herein), is heterozygous (only one allele comprises the “I50V”/“I75V” SNP) or comprises two “wild-type” alleles of IL4R in relation to the “I50”/“I75” position, i.e. is (wild-type) “I50I”/“I75I”. It is understood by the person skilled in the art that the present invention provides for a specific “point mutation” in the IL4R gene which is diagnostic for (erosive) joint destruction in a human patient. Accordingly, a human individual assessed for said point mutation is either “homozygote” for the mutant allele “V” (both genes comprise “V” on position 50/75 of the amino acid sequence of IL4R as defined herein), homozygote for the allele “I” (both genes comprise “I” on position 50/75 of the amino acid sequence of IL4R as defined herein) or “heterozygote” (i.e. one gene comprises “V” on position 50/75 of the amino acid sequence of IL4R, whereas the other gene comprises “I” on position 50/75 of the amino acid sequence of IL4R as defined herein).

In context of this invention it was surprisingly found that the predictive value of the herein detailed and explained “I50V” (“I75V”) SNP or point mutation of IL-4R (CD124) for erosive disease was not only much higher than that of rheumatoid factor (RF) or the shared epitope (SE) but that this specific point mutation in the allele has also (in contrast to the evaluation in the prior art) a very informative character for predicting the progressive erosive disease, in particular in patients who suffer from rheumatoid arthritis (RA). The present invention also provides for the surprising finding that no association between I50V (I75V) and Q551R IL-4R SNPs and disease (RA) susceptibility was identified. No differences in the genotype and allele frequencies of the I50V or Q551R SNPs were determined between RA patients (471 individuals) and control individuals (371 healthy control persons). Nevertheless and surprisingly, the I50V (I75V) SNP is strongly associated with the mostly fast development of joint destruction, i.e. there is a surprising difference between the erosive and the non-erosive group of patients. Therefore, the I50V/I75V SNP has, in contrast to the previously described Q551R SNP of IL-4R, a highly predictive value in particular for the early onset or potential early onset of erosive disease as well as for the erosive character of the disease. Furthermore, as documented in the appended examples it was found that the combination of the valine 50 (V50) allele with either other predictive markers resulted in an increase of the predictive power whereas the combination of for example rheumatoid factor (RF) and shared epitope (SE) alleles did never reach corresponding values. Accordingly, the predictive value of the RF as well as the SE alleles in combination is much lower than the predictive value of for example the (mutant)V50 homozygocity in IL-4 receptor. Therefore, with the teachings of the present invention, at risk rheumatoid arthritis patients can be identified at a very early phase of the disease and corresponding early medical intervention/therapy can be applied. It is immediately evident for the skilled artisan that the means and methods provided herein are not limited to the predictive value of the detection of the herein identified “V” point mutation (“V50” or “V75”). In contrast, also the determination of the wild-type allel (I on position 50 or 75 of the herein defined IL4R amino acid sequence or corresponding natural occurring isoforms and variants) is indicative for the erosive or potentially erosive character of the disease. Accordingly, when a human patient is characterized as comprising the wild-type alleles (“150” or “175”) in the herein defined position of IL4R, a less aggressive or moderate therapy/medical intervention is indicated since in this patient the joint destruction is less erosive or even non-erosive.

As mentioned above and illustrated herein, an association analysis of IL4R with RA susceptibility was performed in a well-characterized case-control cohort of 471 RA patients and 371 healthy controls. Two single-nucleotide polymorphisms (SNPs) (I50V and Q551R) located in the coding region of IL4R (CD124) were examined by allele-specific polymerase chain reaction using FAM-labeled allele-detecting probes. Patients, from whom radiographs of feet and/or hands were available at two years of disease duration (DD), were further stratified according to the radiological outcome into an erosive and a non-erosive group to evaluate the association of the IL4R SNPs with disease progression. In an in vitro cell culture system the response of CD4 T cells from healthy individuals with known IL4R genotypes to IL4 was assessed with regard to the inhibitory effect of IL4 on Th1 cell differentiation to provide a possible mechanism underlying the association.

In accordance with this invention no differences in the genotype and allele frequencies of the investigated SNPs were determined between RA patients and healthy controls. In contrast, significant differences between the two groups of patients stratified according to the presence of radiographic joint destruction at two years DD were observed in the distribution of I50V (processed or mature protein) IL4R SNP genotypes (χ² 15.68, p=0.0004). 68.1% of the V50 compared to only 37.0% of the 150 homozygous patients showed radiographic erosion at two years DD (OR 3.86, 95% CI 1.95-7.64, p<0.0001). This association was independent of individual factors previously associated with severe disease, such as rheumatoid factor (RF) or shared epitope (SE). The predictive value of the V50 homozygosity for the development of joint erosion had a specificity of 0.85, and in combination with either other investigated predictive marker, SE or RF, the specificity further increased. V50 homozygosity had a higher predictive power for early erosive disease than RF and SE. Moreover, homozygosity for V50 SNP lead to a diminished inhibition by IL4 of Th1 cell differentiation and of IL12Rβ2 expression on CD4 T cells. Nevertheless, it can also be demonstrated that in a heterozygous V50 situation the V50 SNP/V50 allele as described herein has a high predictive value.

This present finding is surprising since it was recently stipulated that in particular the herein defined isoleucine to valine mutation at position 50 of the mature, wild-type IL-4 receptor protein (corresponding to position 75 in the herein shown protein sequence derived from the wild type cDNA nucleotide sequence and comprising a “signal peptide” of 25 amino acids; see SEQ ID NO: 2) has no predictive value for rheumatoid arthritis, in particular early rheumatoid arthritis (RA). In particular, Goronzy (2004) Arthritis & Rheumatism 50, 43 stressed that only a single SNP in the uteroglobin gene has predictive value and concluded that further SNPs, also comprising the herein defined “I50V” (or “I75V”) mutation or variant are not informative.

In contrast to Goronzy (2004, loc. cit.), the data provided herein clearly identify the I50V SNP of IL4R (CD124) as a reliable genetic marker for joint destruction, in particular in RA. The herein defined point mutation or “SNP” has a high predictive value for the erosive character of the disease and, i.e., for early joint destruction. This is of particular significance in the clinic and in a medical setting to identify patients with accelerated joint destruction early before the development of rheumatoid damage.

The term “IL4R” as used herein relates to the interleukin-4 receptor and may be abbreviated herein as “IL-4R” and “IL4R”, as well as “IL-4 receptor”. The term is well known in the art and in the present invention it is of note that the position of nucleotides and/or amino acid residue position relate to the known α-chain of the IL-4R/IL4R. The IL4R is also known in the art as “cluster of differentiation 124” or “CD124” and, accordingly, also in this invention the term “CD 124” is employed as synonym for IL4R, in particular for the IL4R α-chain.

The term “mutation in position 465 of the cDNA sequence encoding IL4R/IL-4R/CD124” relates to position 465 of the SEQ ID NO: 1 as provided herein below.

| (SEQ ID NO: 1) | |
|---|---|
| 1 | ccccgcgcgg cgcgggccag ggaagggcca cccaggggtc ccccacttcc cgcttgggcg |
| 61 | cccggacggc gaatggagca ggggcgcgca gataattaaa gatttacaca cagctggaag |
| 121 | aaatcataga gaagccgggc gtggtggctc atgcctataa tcccagcact tttggaggct |
| 181 | gaggcgggca gatcacttga gatcaggagt tcgagaccag cctggtgcct tggcatctcc |
| 241 | caatggggtg gctttgctct gggctcctgt tccctgtgag ctgcctggtc ctgctgcagg |
| 301 | tggcaagctc tgggaacatg aaggtcttgc aggagcccac ctgcgtctcc gactacatga |
| 361 | gcatctctac ttgcgagtgg aagatgaatg gtcccaccaa ttgcagcacc gagctccgcc |
| 421 | tgttgtacca gctggttttt ctgctctccg aagcccacac gtgtatccct gagaacaacg |
| 481 | gaggcgcggg gtgcgtgtgc cacctgctca tggatgacgt ggtcagtgcg gataactata |
| 541 | cactggacct gtgggctggg cagcagctgc tgtggaaggg ctccttcaag cccagcgagc |
| 601 | atgtgaaacc cagggcccca ggaaacctga cagttcacac caatgtctcc gacactctgc |
| 661 | tgctgacctg gagcaacccg tatccccctg acaattacct gtataatcat ctcacctatg |
| 721 | cagtcaacat ttggagtgaa aacgacccgg cagatttcag aatctataac gtgacctacc |
| 781 | tagaaccctc cctccgcatc gcagccagca ccctgaagtc tgggatttcc tacagggcac |
| 841 | gggtgagggc ctgggctcag tgctataaca ccacctggag tgagtggagc cccagcacca |
| 901 | agtggcacaa ctcctacagg gagcccttcg agcagcacct cctgctgggc gtcagcgttt |
| 961 | cctgcattgt catcctggcc gtctgcctgt tgtgctatgt cagcatcacc aagattaaga |
| 1021 | aagaatggtg ggatcagatt cccaacccag cccgcagccg cctcgtggct ataataatcc |
| 1081 | aggatgctca ggggtcacag tgggagaagc ggtcccgagg ccaggaacca gccaagtgcc |
| 1141 | cacactggaa gaattgtctt accaagctct tgccctgttt tctggagcac aacatgaaaa |
| 1201 | gggatgaaga tcctcacaag gctgccaaag agatgccttt ccagggctct ggaaaatcag |
| 1261 | catggtgccc agtggagatc agcaagacag tcctctggcc agagagcatc agcgtggtgc |
| 1321 | gatgtgtgga gttgtttgag gccccggtgg agtgtgagga ggaggaggag gtagaggaag |
| 1381 | aaaaagggag cttctgtgca tcgcctgaga gcagcaggga tgacttccag gagggaaggg |
| 1441 | agggcattgt ggcccggcta acagagagcc tgttcctgga cctgctcgga gaggagaatg |
| 1501 | ggggcttttg ccagcaggac atgggggagt catgccttct tccaccttcg ggaagtacga |
| 1561 | gtgctcacat gccctgggat gagttcccaa gtgcagggcc caaggaggca cctccctggg |
| 1621 | gcaaggagca gcctctccac ctggagccaa gtcctcctgc cagcccgacc cagagtccag |
| 1681 | acaacctgac ttgcacagag acgcccctcg tcatcgcagg caaccctgct taccgcagct |
| 1741 | tcagcaactc cctgagccag tcaccgtgtc ccagagagct gggtccagac ccactgctgg |
| 1801 | ccagacacct ggaggaagta gaacccgaga tgccctgtgt cccccagctc tctgagccaa |
| 1861 | ccactgtgcc ccaacctgag ccagaaacct gggagcagat cctccgccga aatgtcctcc |
| 1921 | agcatggggc agctgcagcc cccgtctcgg cccccaccag tggctatcag gagtttgtac |
| 1981 | atgcggtgga gcagggtggc acccaggcca gtgcggtggt gggcttgggt cccccaggag |
| 2041 | aggctggtta caaggccttc tcaagcctgc ttgccagcag tgctgtgtcc ccagagaaat |
| 2101 | gtgggtttgg ggctagcagt ggggaagagg ggtataagcc tttccaagac ctcattcctg |
| 2161 | gctgccctgg ggaccctgcc ccagtccctg tccccttgtt cacctttgga ctggacaggg |
| 2221 | agccacctcg cagtccgcag agctcacatc tcccaagcag ctccccagag cacctgggtc |
| 2281 | tggagccggg ggaaaaggta gaggacatgc caaagccccc acttccccag gagcaggcca |
| 2341 | cagaccccct tgtggacagc ctgggcagtg gcattgtcta ctcagccctt acctgccacc |
| 2401 | tgtgcggcca cctgaaacag tgtcatggcc aggaggatgg tggccagacc cctgtcatgg |
| 2461 | ccagtccttg ctgtggctgc tgctgtggag acaggtcctc gccccctaca acccccctga |
| 2521 | gggccccaga cccctctcca ggtggggttc cactggaggc cagtctgtgt ccggcctccc |
| 2581 | tggcaccctc gggcatctca gagaagagta aatcctcatc atccttccat cctgcccctg |
| 2641 | gcaatgctca gagctcaagc cagaccccca aaatcgtgaa ctttgtctcc gtgggaccca |
| 2701 | catacatgag ggtctcttag gtgcatgtcc tcttgttgct gagtctgcag atgaggacta |
| 2761 | gggcttatcc atgcctggga aatgccacct cctggaaggc agccaggctg gcagatttcc |
| 2821 | aaaagacttg aagaaccatg gtatgaaggt gattggcccc actgacgttg gcctaacact |
| 2881 | gggctgcaga gactggaccc cgcccagcat tgggctgggc tcgccacatc ccatgagagt |
| 2941 | agagggcact gggtcgccgt gccccacggc aggcccctgc aggaaaactg aggcccttgg |
| 3001 | gcacctcgac ttgtgaacga gttgttggct gctccctcca cagcttctgc agcagactgt |
| 3061 | ccctgttgta actgcccaag gcatgttttg cccaccagat catggcccac gtggaggccc |
| 3121 | acctgcctct gtctcactga actagaagcc gagcctagaa actaacacag ccatcaaggg |
| 3181 | aatgacttgg gcggccttgg gaaatcgatg agaaattgaa cttcagggag ggtggtcatt |
| 3241 | gcctagaggt gctcattcat ttaacagagc ttccttaggt tgatgctgga ggcagaatcc |
| 3301 | cggctgtcaa ggggtgttca gttaagggga gcaacagagg acatgaaaaa ttgctatgac |
| 3361 | taaagcaggg acaatttgct gccaaacacc catgcccagc tgtatggctg ggggctcctc |
| 3421 | gtatgcatgg aacccccaga ataaatatgc tcagccaccc tgtgggccgg gcaatccaga |
| 3481 | cagcaggcat aaggcaccag ttaccctgca tgttggccca gacctcaggt gctagggaag |
| 3541 | gcgggaacct tgggttgagt aatgctcgtc tgtgtgtttt agtttcatca cctgttatct |
| 3601 | gtgtttgctg aggagagtgg aacagaaggg gtggagtttt gtataaataa agtttctttg |
| 3661 | tctctttaaa aaaaaaaa |

This sequence for the “transcript variant 1” is also accessible under NM_{—}000418 (gi: 56788409) provided by the NCBI database. SEQ ID NO: 1 corresponds to the wild-type cDNA sequence encoding IL-4R, i.e. the IL-4R α-chain. The variant/SNP described herein and to be employed in the diagnostic (ex vivo or in vitro) methods and the corresponding inventive uses and means relates to the exchange of isoleucine (encoded in the wild-type IL4R by the codon/triplett “auc”/“atc”) to valine (encoded by het codon/triplett “guc”/“gtc”). However, as laid down herein, also the determination of homozygous or heterozygous wild-type sequence is of diagnostic value, in particular for the attending physician. The diagnostic value of the wild-type IL4R in the present case is the fact that a patient who suffers from rheumatoid arthritis and has, however, a wild-type IL4R (on the position I50/I75 as described herein or in the corresponding encoding nucleotide sequence) requires a different medical treatment regime than a patient who has the herein described point mutation. A patient suffering from rheumatoid arthritis (RA) but being homozygous for the wild-type allele I50/I75 can be treated less aggressively than a patient who has either homozygously or even heterozygously the mutant allele (V501V75) described herein. Cases with at least one mutant (I50→V/I75→V) allele merit aggressive treatment in order to prevent joint destruction or accelerated joint destruction, whereas (conversely) cases with the wild-type allele, in particular with homozygous wild-type alleles require and allow for a more conservative, less aggressive medical intervention/medical management. Accordingly, it is also part of this invention that in the herein described means and methods the wild-type situation in the herein described “SNP” (I50 I/I75 I) is evaluated. The wild-type ILR4 referred to herein encodes for a polypeptide as shown in the following SEQ ID NO: 2

(SEQ ID NO: 2)

MGWLCSGLLFPVSCLVLLQVASSGNMKVLQEPTCVSDYMSISTCEWKMNG
PTNCSTELRLLYQLVFLLSEAHTCIPENNGGAGCVCHLLMDDVVSADNYT
LDLWAGQQLLWKGSFKPSEHVKPRAPGNLTVHTNVSDTLLLTWSNPYPPD
NYLYNHLTYAVNIWSENDPADFRIYNVTYLEPSLRIAASTLKSGISYRAR
VRAWAQCYNTTWSEWSPSTKWHNSYREPFEQHLLLGVSVSCIVILAVCLL
CYVSITKIKKEWWDQIPNPARSRLVAIIIQDAQGSQWEKRSRGQEPAKCP
HWKNCLTKLLPCFLEHNMKRDEDPHKAAKEMPFQGSGKSAWCPVEISKTV
LWPESISVVRCVELFEAPVECEEEEEVEEEKGSFCASPESSRDDFQEGRE
GIVARLTESLFLDLLGEENGGFCQQDMGESCLLPPSGSTSAHMPWDEFPS
AGPKEAPPWGKEQPLHLEPSPPASPTQSPDNLTCTETPLVIAGNPAYRSF
SNSLSQSPCPRELGPDPLLARHLEEVEPEMPCVPQLSEPTTVPQPEPETW
EQILRRNVLQHGAAAAPVSAPTSGYQEFVHAVEQGGTQASAVVGLGPPGE
AGYKAFSSLLASSAVSPEKCGFGASSGEEGYKPFQDLIPGCPGDPAPVPV
PLFTFGLDREPPRSPQSSHLPSSSPEHLGLEPGEKVEDMPKPPLPQEQAT
DPLVDSLGSGTVYSALTCHLCGHLKQCHGQEDGGQTPVMASPCCGCCCGD
RSSPPTTPLRAPDPSPGGVPLEASLCPASLAPSGISEKSKSSSSFHPAPG
NAQSSSQTPKIVNFVSVGPTYMRVS

This sequence depicted herein above comprises in position 75 an “isoleucine” (I); wild-type sequence with 25 amino acid signal sequence. Said “I” is in the mutant SNP described herein replaced by a “valine” (V). Said point mutation is employed in the inventive methods, means and uses described herein and is a diagnostic marker for joint destruction, i.e. the prognosis or the progression of joint destruction. In particular, in case when such a point mutation is present in a human individual/human patient, it is indicated that this patient is treated in a more aggressive fashion in order to slow down and/or ameliorate joint destruction(s), in particular early joint destruction(s). The present invention is of high importance since an early intervention with medicaments improves the outcome in joint destruction/joint erosions in particular in rheumatoid arthritis. However, in accordance with the present invention, the attending physician can deduce whether an aggressive or less aggressive/moderate medical intervention is indicated in a given patient. The present invention is particularly useful in the deduction of patients who will suffer from a more prominent and/or early joint destruction/joint erosion. If a patient has the herein described point mutation, the likelihood to suffer from early joint destruction is much higher and said patient requires more aggressive and early treatment.

The sequence provided herein above as SEQ ID NO: 2 comprises the encoded IL4R α-chain and an additional original signal peptide of 25 amino acids. Accordingly, the SNP or variant described herein relates to an amino acid exchange from isoleucine to valine, which can be depicted and illustrated as “I75V” (relating to the polypeptide comprising the additional 25 amino acid signal sequence) and as “I50V” (relating to the encoded polypeptide without signal sequence).

The person skilled in the art also finds IL4R coding regions (located on human chromosome 16) under the gene accession number X52425 (gi: 33833) or under AF421855 (gi:15987825)

In the following SEQ ID NO: 3, the complete genomic DNA of the human IL4R gene is shown and in positions 5758 to 5760 the variant nucleotide sequence “gtc”/“guc” is shown which leads to the encoded I50V/I75V variant.

| (SEQ ID NO: 3) | |
|---|---|
| 1 | tacaggtgtg agctactgtg tctggcctga ataataaaat ttaaaacaat ttttcaaaaa |
| 61 | ttcaccatga ggtctcacta tattccctag gctggtctca aacccctgga ctccaagtga |
| 121 | tccaccccac cttcccgagt agctgggact agagatgcac accattgcac ccaatagagc |
| 181 | aatacgtttc tgttctttgt aaattacctg ctctaaggta tttttgttat agcagcctgt |
| 241 | atggactaag ctgacttgta acgttacttg agactttaaa gtgttccggt cactgttgga |
| 301 | gggctctgtc tgtgttagct catttaatcc ccacaacacc tcaatcagat ggggctattc |
| 361 | ttagtcccac tttatagata aggaaactga ggcatggaag cacagcttgc tcaaggttca |
| 421 | catctagtca gtgacagagc aggtatttaa acctcaggaa ataatcagag aaacatgtgt |
| 481 | agagggttgt ccaaggaagg ccacatccag aagcatctcc caggacagtt gttgtgtagc |
| 541 | tcaccctctg gactttgtgg gtctgggtgt tgtttcatga ttatagagag agctctgtga |
| 601 | acgtggagga cctgttgtcg gcagagacac aaatggccag ggcatggctg ggcagccgca |
| 661 | gtggctcagg cctgtaatcc cagcacttcg agaagaccag aggggcagat catgaggtca |
| 721 | gaagttcaag accagcctgg ccaacatggt gaaaccccgt ctctactaaa aatacaaaaa |
| 781 | ttagccaggt gtggtggtgg gcacctgtaa tcccagctac tcgggaggct gaggcagaag |
| 841 | aatcgcttga acccgggagg tggaggttgc agtgagctga gattgcaccg ctgcactcca |
| 901 | gccttggaga cagagcgaga ctctgtctcg gaaaaacaaa caaacaagca aacaaacaaa |
| 961 | caaataaatg gccagggcag gggagggttg catattgaat aagatgagct ctgctggaag |
| 1021 | cacaggtcag cactaacctg cttcctctct ctctgcaggt gccttggcat ctcccaatgg |
| 1081 | ggtggctttg ctctgggctc ctgttccctg tgagctgcct ggtcctgctg caggtggcaa |
| 1141 | gctctggtaa gtcaccactt ctcaatcatt catttgttgg ctattaatgg cgtgccaggg |
| 1201 | tcctgcagta tgtcacctgg ccttatggag attacactgc agtgggaggg gacagccaat |
| 1261 | gacaagtggc cctgattatc agtaaattct aaagattgtt agaaagtgat gggagccggg |
| 1321 | tgcagtggct cacacctgta atcccagcac ttcaggaggc cgaggcagga ggatcgcttg |
| 1381 | agcccaggag ttcgaggtca gcttgggcaa catagggaga ccttgtctct acaaataata |
| 1441 | aaatattagc caggtgtggc agtgcacgcc tgtagcccca gctactcagg aggccgaggt |
| 1501 | gggaggatcc cttgaactca ggaggtcaag gctgcagtga actgtgatcg cgccactcca |
| 1561 | ctccagcctg cgtgagaaag tgagaccctg tcaaaaaaaa agagaaggtg atggggaaag |
| 1621 | aacacagaac agcataagag ggggttgggg aagctgggtg gagtgggggg gattgcagtt |
| 1681 | gaaagtaggg aagtcaggga aggcctcatt gagctgactt ggaggaagcg ggaaccgtgc |
| 1741 | agatgtctgg ggaaggctca ttcttggcag agaggccctg cactgagcct ggcgggaggg |
| 1801 | ttgagcacag gagggaatgt ggtggaggag agtgagcagc aggagggagc agtgaaggtc |
| 1861 | agcaaggtga cagagtggct gaatcaaaaa agaccttgca gtgtttgagc agaggatcca |
| 1921 | tatcatccat tatgttccaa aggactcttc aggatgccgt gtggagaaag gaagagggtg |
| 1981 | gaagccagga ggtctggagg gaggtctgga gtggaggaga tgagaggctc cggatccctc |
| 2041 | tgggaggtag atttgaggac agattggaat tgaggtgaaa gacagagaaa gagaagtggc |
| 2101 | caggatgact ccaagatttc tgacctaaac tactgggaag gacgcggttg tcatttctga |
| 2161 | aatgcagaag gatgccagaa gagaaggtac tttggggagg ggcgggaatc aggagttagt |
| 2221 | tttggacatg agataagctt ggaatattta tttgctatct aagacagctc cttaacatgg |
| 2281 | taagccctta tgcaagttgt tgtcagctga gatgggcgtg gcactgagca tgggagcatg |
| 2341 | gaggcgcctg agtggtctca tgctcaggtg gtttagcaaa ctcagtgtac atcctgccaa |
| 2401 | ttccagtcct gccatggcca ctgacaagct aggagggcgc tgaaaggaga aggaccccga |
| 2461 | tgtctcctcc agcccatcca tctcctctct cccattggcc aaacccaacc ggaaactaaa |
| 2521 | ggccaagggt acccggtgat gaagactgtg gtatcagcct cctgagcaca gagagggcag |
| 2581 | aaaggggtgg agacaaagag gggcgcagat agtgggcaaa tggggaagtg gcacttcccc |
| 2641 | tagctcgagg gcagaggctt ggtgtgatgg aatggcactc cttaaactgc tacatatttt |
| 2701 | ccctttaatt tggccaagaa caagttgtca agtttgtgtg agataaaggt gcacttggtt |
| 2761 | cgttcttgtc taatggcccc cgcacccatg ggtatttctt cagcttccac agtcatcccg |
| 2821 | acactagctg ggaagctcca gcagccctgg tcctggcccc agctctgtgg gcgctggccc |
| 2881 | tcaactttgc ctgcactgtg cttttgtgct attccccttg gtcctgtttg ggtgcaagtc |
| 2941 | cccctcacgc attgagttcc tgggccgctc aggctgctcc tgtgtctccc cagggaacat |
| 3001 | gaaggtcttg caggagccca cctgcgtctc cgactacatg agcatctcta cttgcgagtg |
| 3061 | gaagatgaat ggtcccacca attgcagcac cgagctccgc ctgttgtacc agctggtttt |
| 3121 | tctgctctcc gagtaagcct gcgctggagc tggaggtttg gggaggttgt gcccaaaggg |
| 3181 | tttgccccaa gagtgagctg ggtccaggtg gtgcgctgga gtgcaggatg ctgagtatgg |
| 3241 | tttgctgctg tttatatggt gttagagggg aggtcccatc tccagggaca tgttatgtaa |
| 3301 | gatacagtgg agcgcatggt gggagtgttg gtccacgtgg cacatggata cggctggaat |
| 3361 | actggactag accagcagtt ctcacacttt ttggtctcag gacccttttt cacacttaaa |
| 3421 | aatgagtgag gacccaaagg gctttggtgt aggtaacaca tcattctatg tttacctaat |
| 3481 | tagaacttgc aatgaagaaa tggtgtaatt tttaaaaaat taaaacaatt aaaaattttt |
| 3541 | tttcttactg aaatggaggt ctcactgtgt tgcccaggct gctctcaaac tcctgggctc |
| 3601 | cagtgatcct cctgcctccg cctcccaaag tgctgggatt acaagcgtga gccgctgtat |
| 3661 | ccggcccaaa atggagaaat tttaagtccc aacaacatgc aagcccgcat tcaacaaatc |
| 3721 | ttcagatcaa ttacatgatc acaggtcatg tagcctctag aaaattccac tgtacgccag |
| 3781 | tgagagagag tgaaaaggca aataacgtcc ctgtattatg atgaaaagag ttttacctgg |
| 3841 | tgggcccaga ccacactttg agaaccactg gactagaccc ttgattgagg agtacggtgt |
| 3901 | tgagagtgga gtcctctgtg atggtggatg gaccaggaca catggcatag gagtcaggtg |
| 3961 | gttccctggg ctactccatg gtgcacagga tgcttcgtta cactggtgcc caggacataa |
| 4021 | tcacgtacac aagacacaca gttacggggc agactgggga tatacggcac accagcatgc |
| 4081 | agggttcacc agtaaaggtg gtattccatg attattctaa ggtagatggg ctgtgctttg |
| 4141 | tttccattgg cttagtccag ggattggcaa actatggccc gtgagccaaa tccggcccac |
| 4201 | tgcttgtttt tgtaaataaa gttttattgg aacacactgg ctgctgtagt tgtaacagaa |
| 4261 | actgcatggc cctcctttat gttttttgtt tgtttgtttg tttgtttgtt tgttttcttt |
| 4321 | gagacagagt ttcgctcttg ttgcccaggc tggagtgcag tggcacaatc tcggctcact |
| 4381 | gcaacctctg cctcccgggt tcaagcgatt ctcctgtctc agcctcccga gtagttggga |
| 4441 | ttaatggtgc ctgccaccac acccggctaa tttttcgtat ttttagtaga gaccggtttt |
| 4501 | catcatgttg gccaagctgg tctcgaactc ctgaactcag gtgatccacc cgcctcagcc |
| 4561 | tcccaaagtg ctgggattac aggcatgagc cactgagccc ggcctcctcc tttatcttaa |
| 4621 | ttgaaataat tcagaaatgg aaagtcaaat actgcatgtt ctcacttata agtaagagtt |
| 4681 | aaataatgtg tacacatggg cattattcca tgtaccatgg aataacagac attgaagact |
| 4741 | tgggagggtg ggagaggggt gaaggaagag aagttactta atgggcatag tgtacaccat |
| 4801 | ttgggtgacg gacccaccag aaccccagac ttcaccacta ggcagcatat ccagtgagaa |
| 4861 | cagatctgag gcttgccatc aaaattgcac ttgtaaggcc gggcactgtg gtggctcgcg |
| 4921 | gctgtaatcc cagccctttg ggaggccgag gtgggcagat cacttgaggt caggagttcg |
| 4981 | agaccggcct ggccaacatg gtgaagctcc atctctacta aaaatacaac aattaactgg |
| 5041 | gtgtagtggc gcacacctgt aatcccagct actagggagg ctgaggcggg agaattgctt |
| 5101 | gagcccagga ggtggaggtt gcagtgagcc gagatcacat cactgtactc tagcctgggt |
| 5161 | gacagtgaga ctttgtctca ggaaaaaaaa acaaaaacaa aaaacaaaaa actcgtaccc |
| 5221 | cctaaattta tacaaataac caaaaaaaaa aaaaaaaaag gaaattgtgt ggctttgaag |
| 5281 | tccaaaatat taactatctg gcctgttaca gaaaaagttt gcagacccct ggcctagccc |
| 5341 | gtgagatgtg ggttggctgt taaggtggaa cattggaatt atcttacgat ggccaaactg |
| 5401 | tgcgatgcag agcttatgtt gttctaaatt aattagtgcc accggttctt ccctttcatg |
| 5461 | ggctttcagg aacaagctaa gtcccaggac cagggccggc agctaggcag gtgtgaggag |
| 5521 | catccttggt gcatgtggta agaggctgtg gccagcaaga gaggcaaccc tagtcggctg |
| 5581 | ccccagcaca ccctggccgc tcccaagccc ccagatctgt cctcacatcc gtgatcggga |
| 5641 | agctggaaga gtctgatgcg gttcctggag gcatgtcccg gacacagctg tggggcccag |
| 5701 | ccagcctaca ggtgaccagc ctaacccagc ccctgtgtct gcagagccca cacgtgtgtc |
| 5761 | cctgagaaca acggaggcgc ggggtgcgtg tgccacctgc tcatggatga cgtggtcagt |
| 5821 | gcggataact atacactgga cctgtgggct gggcagcagc tgctgtggaa gggctccttc |
| 5881 | aagcccagcg agcatggtga gcagggcgga gtgcggcagg ggtggctggg tgtgttccca |
| 5941 | cagctgcctg ggctgagggt ggggtgggca ggggaggagg tggggtcata gcaacagcag |
| 6001 | gaggaagccg cctgtatttt cccaaatctg atgggattcc tgcccctgcc tgggcctcag |
| 6061 | tcctcccacc tttgaaacgg agctggtcgc agtagaccac caagccccct tcagcccagc |
| 6121 | tgtttccacc cctgaactta agtgcccagg aaggcgtatt gagatgaggt gtgcttgctg |
| 6181 | gaaggcatgc ctgctgctga ttgaaaaccg aactgggaac attccttcca ttctgtgtcc |
| 6241 | actggtcagc tgctgcggct ttggatggtc ttgaccgtgg aaggctgacc ttcttctggt |
| 6301 | acccggagtc cctgcaggaa tcccccttga gcttgctggg ctgtggtgac aggagtttaa |
| 6361 | aacatgcgtt gtattccagt gatgcatgat atgacatgca tcacaggaat aaaaacctga |
| 6421 | ggtctcatgg atatgattgc ttcaaaggag accaagtttt aaaacagatg aatcaaaata |
| 6481 | aagaaaaata ctcagtaaat catcataaag tacagagatg tggccaaagg tgtgaaggat |
| 6541 | gcagctgtaa aagctgaagt ttgaggccgg gtgtggtggt tcatgcctat aatcccagca |
| 6601 | ctttgggagg ccgagcccag cggatcaccg gaggtcagga gttcgagacc agcctggaca |
| 6661 | acatggtaaa accccgtctc tactaaaaat acaaaaaatt agtctggcat ggtggcaggc |
| 6721 | gcctgtaatc ccagctactt gggaggctga ggtaggagaa tggcttgaac ccaggagaag |
| 6781 | gaggttgcag tgagcttaga tcatgctact gccctccagc ctgggcgaca gagtgagatt |
| 6841 | acgtctcaaa aaaataaaaa taaataaaaa taaaaagatt ttttaaaagg ctgaagtttg |
| 6901 | ggttactttg gctcatacac tttgccttca ctgtagaaag gtggttagta aagaccaggc |
| 6961 | gcggtggctc atgcctggaa tcccagcact ttgggagccc agcgcaggca gatcacttga |
| 7021 | gccctgggct attgaggctg cagtgagctg ggattgtgcc actgcactcc agcctgggca |
| 7081 | acagagtggg accctgtctc aaaaaagaag aaaaaaaggg taattaataa acactaaagt |
| 7141 | tctatgtaga attttagcaa cattattgtt attataatct tctttgctat ggctctgaat |
| 7201 | ctgtgtggtg ctccagaagt atgctatgga ggttttgtcg accaaaaatc tgggtggtgg |
| 7261 | ctgtggtttg taggccgggg ctgggctggg tgatggggga gtcactgcat agatcctcac |
| 7321 | atagaggccg cttctcccgc agtgaaaccc agggccccag gaaacctgac agttcacacc |
| 7381 | aatgtctccg acactctgct gctgacctgg agcaacccgt atccccctga caattacctg |
| 7441 | tataatcatc tcacctatgc agtcaacatt tggagtgaaa acgacccggc agatgtgagt |
| 7501 | gggcatgctt tgacgttttt ctgtgacctc tggggaacag ggtgggtgac cagcagaggc |
| 7561 | ccagtccctg gagccaggag cctgggaggc aagccctggg gctggatagc aaatcccagg |
| 7621 | agctagagac ctggcttctc acctggctct gccctaggca agtccctttg cttcctggcc |
| 7681 | ccccacccct cacatcagag aaggggagtt atctctgcat gccgctcctc ctctgtaaag |
| 7741 | gtagggctgt gggccacatc tgtgtttccc agtttggggg acacaagtga tcgtaggtgg |
| 7801 | cacattgaca gctcacttga ataaccctat tattgaagag aataatactg actcaagaga |
| 7861 | cagtgacccg tgtcagttcc cttttgaggc caacgggtta aggaggaagt ccccatacag |
| 7921 | ctgactcgtt tactaattcc tcttaatgaa gagagcagag gccacacccc aggcttagac |
| 7981 | tttcccaaga aaacaagatc agtttgttgg ttgttcccca tggaagctgg tcctgacatt |
| 8041 | cccttcacag tagtgttggt ggagtttttg ttgttgtttg ttttgagaca gagtctcact |
| 8101 | ctgtcaccca gggtggaaca cagtggcgtg atcttggctc actgcaacct ccgcctcctg |
| 8161 | ggttctagcg attctcctgc ctcagcctcc tgagcagccg ggactacagg cacctgccac |
| 8221 | cgtgcccagc taatttttgt atatttagta gagatggggt ttcactgcgt tggccaggct |
| 8281 | ggtctcaaac tcctgacctc agatgatcca ctcgcctcgg cctcccaaag tgctgggatt |
| 8341 | acaggtgtga gccaccgcac ctggccagtg gagttccttc ttaagtacat gtattgacat |
| 8401 | ctttaaaaag ggcgagagga tttacaggaa actatcaggt cagtaatggc aggggccgtc |
| 8461 | cacagtgggt ggctgagtcc ccctattttt ctgctggtgt gcggggaggt catttcctgc |
| 8521 | cacccatgtt tccccaccct gaatccacct tcctcacatt cccattggag ggacactctc |
| 8581 | tggacatatg ggacctgggg tcccacaggg ctgcattcca atgcctgctg tgccactcgc |
| 8641 | cagctgtgtg atgttgggca tatcccataa cctctttgtg cctcagtttc ctcatctgta |
| 8701 | acacaggagt gacaagagca cccgcccaca gggctatgac agtacaaggt gtgtgataca |
| 8761 | gatgagctcc cctgtttggc ccacatgtgt cctaaaagcc atgtgccctt tctcttgagt |
| 8821 | gccccaggcc acagagatcc ccatctgccc gctgtcccac acactggtct gtcatttgtt |
| 8881 | ccttgaggtt tgtgagggcc ggctctgtgc atcccagggg cccaggctgg gcctggttgg |
| 8941 | ctctcaggga gcaggcaccc gccaccttaa gctcccatgc tggtgtctgt cactgcttcc |
| 9001 | tctcaatctg gccaagccag gggtgtcgat ttatatctct caggtctggt ttcccctttg |
| 9061 | gcactgggcc aggtatgggg aaagagcagg aatggggcag ttggctcaca cagcagaggc |
| 9121 | tcagaaagcg gggggcatgg ggggaaggag tgcacagatg ctagagagtg gggcaagttt |
| 9181 | tgtttggtca ataaatctcc ttctcatgcc ccaggcctgt gcaagaccta cagagagtcc |
| 9241 | caaggatggg ctggggggaa gagaaaggta ccaccttcag agtccaaaga tatgttattt |
| 9301 | aatattttca tatttctaga tctgccttca ggcatggctg gatccagctt ctaggaacct |
| 9361 | gtccagctct gcgccctgct ttattctgta ctggcttcgt ttttaggcag gctcttccct |
| 9421 | catgtagtgg cagatatgcc tactagttgc tccaggccta catcccaaag ccacagtggg |
| 9481 | aaaagggttt tttttcttga cggttctaat aagagtccta aggctgctgc tcagtggcct |
| 9541 | ggcttcgatg ctgtgccagc ctctgaacca atcactggct gtgggtggag agagggtgct |
| 9601 | ggtggagggc cctgcttgtc cagggaggag tcacatacct gcctctaggg ctgcaggtgg |
| 9661 | gctcagctcc atccaaacca gatgaactga aaataaggca ggagtggctt ccccagggga |
| 9721 | aactggggaa gaggaagcag gactgtgctg gctaaaatgc cagccaggtt taagacgtgg |
| 9781 | caccagatgc cagtcatggg attggattgg tcagcatgcc tgggctatgg cttaggggta |
| 9841 | tgttggtgct cagggatgcc acaggcctcc agataccagg tctgaggcag aagaatgaag |
| 9901 | tccagcttct cttgtgggtg gaacagtggc aactgagata ccccatctct cccttcccaa |
| 9961 | gaacagagct gaacataaag aatttagtga ttggccagag cttggccaca tgctcccctc |
| 10021 | tgatgaatga taggccaggt gatgggattg gcacaattgg cttagactaa tgagggttgg |
| 10081 | ccctggagtt gcaggcagtg gagttctgtc ctaagcagtg ggcacctaaa cccgatggca |
| 10141 | taaaagctgg gcgggtgtcc acctgcatct gccacagcac tgcaggcacc aactgtggct |
| 10201 | catactgagt gggataaatt ccagaaagaa acattaggaa cttactatag aattttgggg |
| 10261 | ctagagctac tcattcattc ccctagataa tttctaggca aggttccata gtggaggggg |
| 10321 | agttttggct tgggcattga aggatgcata ggagttttct agatggggaa agaagggaac |
| 10381 | ggtagaccag gcagagggaa ctgcatgata aaaggtttat gggtgtgaaa attcatggaa |
| 10441 | tgtttgagga ttatggggtt gggggatgtg ggaatatgtg tagcgataaa gcaccaaaca |
| 10501 | aagccaaaag tttagttaga gccctgaatg cctgcctcat aatggtttcc atattttata |
| 10561 | tgcctactat gtgccaggca cattgctcag ggtcacacag ctggaaatgg cagggctgag |
| 10621 | tttttgttgt tgttgttgtt gttgttgaga cagagtctca ctctatcacc caggctggaa |
| 10681 | tgcaggggcg tgatcatggc tcactgcatc cttgacttcc tgggatcagg tgattctccc |
| 10741 | acctctgcct cccaggtagc tgggactaca ggcacaggcc accacgccag gctaattttt |
| 10801 | tgtattttta gtagcgacag ggtctcgcca tgttgtccgg gctggtctgg atctcctggc |
| 10861 | ttcaagtgat ccccctggct cagcctccca aggtgctggg attacaggct tgagccaccg |
| 10921 | catccagccc agatctgaga tttgcaccca gtatttgaac tcccaagcct gtgctctttt |
| 10981 | tcctcccatg gacatttctc tcagagatgg tctcccaaac acctgtcctt cttgttaaaa |
| 11041 | aacagacaaa ccgcaagtag ttctttggaa gctcagattt ctcttttgtt tcttagtaaa |
| 11101 | acatttccca gttcccagct cccttccagg gtgtaagatt tcttcggtaa cttacatcta |
| 11161 | gctgttgctt cttgtttgct catgtttaga aagaaagaca aaagagagtg agaattttct |
| 11221 | ctcccttccc cagtctcccc acaactcaca ccccaccctc agctccctct gtaataggaa |
| 11281 | aatctctgaa ctctctgtag ttgctccagc aatcttttgg aactttgctt ctttcttgtg |
| 11341 | aaaaaacctc cccttggctc actttgcacc aggtttcccc aaatgtgctt ccaaccacaa |
| 11401 | gcagaaatgg agctgccagt aaccaggaag aaactgccgg gggctgagga agaggagagg |
| 11461 | gaggtgcata gccctggatc tcgcagggag aggggtgaca ggatgagaac tcaggttgct |
| 11521 | cacttgccat cagggtcagt catgaatata gcgttcatgt atcacttttt aaagcttttt |
| 11581 | tggagggtaa aagtaatagt tacacaaaat aaaaatacaa atggtacaaa aggacttaga |
| 11641 | atggaaacat gtttctctcc cgactccagc ctcctgtttt tcttcccaga gactgaccac |
| 11701 | tgctgtctgt ctcttgccag aagggaaagg gaggcaaggt tagggcaggc agagggcatg |
| 11761 | tgcatccttt agagagagct tatgtctata caagcaaatg tgtgtgttca gtcatcgctg |
| 11821 | tcttagtttt ctattgctgc ataataatgg tactaccagc ttcacagctt taaacaacac |
| 11881 | ccatttatta tctcatagtt tctgtggttg ggagtctgga catagcttag ccaggttctc |
| 11941 | tgctttagag tctcgtgagg ctataatcaa ggtgtgggat ggggctgcag tttcatctga |
| 12001 | ggctcaattg gggaagggtc acttctaagc tcatacaata ttggtgacat tcagtccctg |
| 12061 | gcaggctgtt gaactgagag cctcagtttc gtgctggctg ttggttgtag ttaaccctga |
| 12121 | attccttccc atgtgccctt tgcaaagcca tcaaggcaga gagacttgcc tagcaagtag |
| 12181 | gatattacag tcttctgtaa tataatcaca tccatgaaat cctctatata tcccatcacc |
| 12241 | tttaccatat tctgtgggtt agaaacaagt agcaggtcct gcccacactc gagaagacca |
| 12301 | gatgacacaa agatgtgatt caaagtgggg atcatcgggg ccatcttagg tttgtctgca |
| 12361 | gtgatcactg tgccatctct ctctctctct ctcttttttt tttttttccg agacgaagtc |
| 12421 | gtcactctgt cacccaggct ggagtgcagt ggcatgatct cagcttacca caatctctgc |
| 12481 | ctcccaggtt caaatgattc ttctgcctca gcctcctgag tagctgggat tacaggtgcc |
| 12541 | cgccaccaca cccagctaat ttttgtattt ttagtagaga cagagtttca ccatgttggc |
| 12601 | caggctggtc ttgaactcct cacctcaagt gatccaccca cttcggcctc ccaaagtgct |
| 12661 | gggattacag gcatgagcca ccatgcccag ccccatctct ctttaaaaaa caaacaaaca |
| 12721 | aacaaaaaac ataaaaagaa gcagagaaca catacacatc tgcatcttcc cttgtttact |
| 12781 | taacaataga tcttggaagt cacttctcag tagaggctag gttgggcaga gcattggatt |
| 12841 | ctaggccagt gagtttggac ttgaccatgg agacactagg aagcccatga aggacagaga |
| 12901 | gagatgcctc gaccctgcca gtcctttaga aagatcaccc agtgcttttt gtataccaaa |
| 12961 | ccctatttga aatacttacg tatattaacc catttcctta tcaccacaac cctgcgggaa |
| 13021 | gggagatagg cacttttatt atcttcattt tgcagatgag gacattgagg tccagagagg |
| 13081 | ttatgtcact tacttaaggt cacacagcca ggaagtggta gtagggactc ttacccttgt |
| 13141 | tttacagatg agattgaatt atctcacgaa aactcagaaa ggttaaacaa cttgcctaag |
| 13201 | taacatacag ctaattagtc gaggagcctg acgcatgttg ctctagcctg gtcacagtta |
| 13261 | cagaggtggc aagcaatggc ctgaacagga cgaacaacca aatacccagg ctggtggctc |
| 13321 | ttaaacatgg tggggtcagc taacgacagc aaccagggtg ggcactggtg cccctcgccc |
| 13381 | ccggctggtg ccctaacatc tcccttttct ctaccagttc agaatctata acgtgaccta |
| 13441 | cctagaaccc tccctccgca tcgcagccag caccctgaag tctgggattt cctacagggc |
| 13501 | acgggtgagg gcctgggctc agtgctataa caccacctgg agtgagtgga gccccagcac |
| 13561 | caagtggcac aactgtgagt atcaagaggc ctaagcaatg gtaatctcca ctctccattc |
| 13621 | ttcccctgtg gccagacact tcccctggct gagtctctgg gcttttatat cataggatgc |
| 13681 | ctctaatggc aatcctgcca ttagatacac ctgccgtggt gtatctgcca ggtaggcagg |
| 13741 | ctaggctgca gtaacacaca agcccacaat ttccatggct taacactata ggaatatatt |
| 13801 | tcttgctcac gtaacaagct aacgtgaatg ttgctggttt gtaggtggtt tccctccctg |
| 13861 | tagaaatctg gggagtgagg ttctttccat cttgtggtgc catcattctc caggacaaag |
| 13921 | attcttacct acttttgtgt cctggtttcc tttggcagcc tggtgaagcc tatggacctc |
| 13981 | atttcagaat atttttaaat acataaaatc ccagcctggg caatatagtg aaacccccat |
| 14041 | ctgtacaaaa attagccagg catggtggca tgcacctgta gtcccaggta ctgggaaggc |
| 14101 | tgaggtggga ggatcacttg agcccaggag tttgaggctg cagtgagccg tgatcgtacc |
| 14161 | actttactcc cacctgggtg acagagcaag agcccatctc taaaaataaa taaatacaat |
| 14221 | gaaataaaat aaaataaaat aaatagaact acagaggaaa ctaattgtat tgaaatgcag |
| 14281 | ttataaaaca tttaaacaca tttttaatct agagatatat gtgcttcttt attaagatct |
| 14341 | ataaataata agttctaggg gtagctcgca taaatactgt aatttcaaag tagataagca |
| 14401 | taaataatac tttatgatac tgaaattgtg atgtgatatg agaatagctg tgagttttgt |
| 14461 | tttgctgggg acaggatcac tgatgctgtc attactgggg tctcttccct ccattctttt |
| 14521 | tttaaaattg tattttattt tatttttaaa attttaaaat aaatagagac agggtatcac |
| 14581 | tatgttgccc aggctgcttt tgacctcctg ggctccagtg atcttcccat cttggcttcc |
| 14641 | caaagtgctg ggattacaag tgggagccag tgttcctggc cccttcctcc attcttaatg |
| 14701 | gaaggagatg ctaggtgtga gaggttaggg aaagtaaaga tgtaatttct ttcccatcca |
| 14761 | agttctcaga cccctgaatt ctacctgcag ccatgttggt ccatcaaccc caagtgaaga |
| 14821 | atccctgctc tagggcccca ccattgtctg tatccagcca gcagaagagg cgtgattatg |
| 14881 | gagatcacat ctgcttcttg aaagcagaca gcccggaagt gggccgcatc acttcctctc |
| 14941 | aaattctatt ggtgaaaatg gtcacatgac tacacatagc cacaaaggag gctgggaact |
| 15001 | ttctcacttg gaacctacat cccagaaaca actcttttca gtgaggtatc ccacaggtct |
| 15061 | ttcgcagtag aaatattgat tatctcacat aaaatgaagt cttacaaatg gacctactgg |
| 15121 | gttttgtaca gcagccaagt gatatctctt cccttctgct gtcttccctt ctgccatcct |
| 15181 | tcacatggtg gcattgtatc cttagacttg ccacccatgc cctcaggttg gccgttgcac |
| 15241 | actgtcttac ataaagcagg aaggaaagga aaggctgcta cgagagagtg taccttgtgc |
| 15301 | atctcttttt taatcaggaa gcaaacatct ttctagaagc ttccctagca aaattcccct |
| 15361 | tacatctcat tggccaagac tgttacatgt tacatggtta ctgttattac ttgctcattg |
| 15421 | caaggaagac tgggaactca aatgcctgga aaaaggaaca ggataatcgt gattggctca |
| 15481 | agccttaggg tgggcatggc tccctgacaa gggagagagg aaaaagctgt tgagtgaaga |
| 15541 | agactgcttc agtttcccca tctgtataat gggaggagta agggctgtcg tgaaaactca |
| 15601 | atgaaagaag attcttcaac gtggtaggtg cagtggcagc tggcagtacc ctgaccctgc |
| 15661 | caccgcacag ccctctcagc attgctcatc ctgcactgtg gatatcagtt gagccacgtg |
| 15721 | tctcctgccc tgggctgtga gctccatagg cagggtctcc atggctgtat ctccagaacc |
| 15781 | cagcacagaa ccaggtgctt gggaaagttt tgaattgatt ctcatctgcc attggcatgg |
| 15841 | ggaagggaac tagcttgtat gaaacagata acaatgtatg ggaccctcat tcattatttc |
| 15901 | agcaaatatt tgctgagttc ctcctacatg gctagccctg tgctagacac tggggaatcg |
| 15961 | gcgatgaaca aagcagatag aaatccccac tcttgtggag ctgacattct ggagggagag |
| 16021 | acaaaaagca aacatataaa gaaagaaaga aatcacatgg atctggatga cagtgagtgc |
| 16081 | tgggaagaaa ataaaagcag aggaagggga tggagcgatg ggcagggggc aacggtaggg |
| 16141 | agggtgtcgg ggaaaacttt ttggagaatg tgacgatgaa agtgaacaag gagaagtcaa |
| 16201 | ccgtgttgag atgatggcag ctaatgatgt ggacaggcca ctctgttctg agtgcattat |
| 16261 | ctattgattc atcatgtcat cctcgcaaca gccctgcacg atcaattctg tcattaaccc |
| 16321 | catagtacag atgaggatgc ggaggcacag agaagataag ggacttgtcc tgtgtcacac |
| 16381 | agcaaggagc catccggctc ctaagttggt gcatttgact tctgtgcttc cggaaagaaa |
| 16441 | gagcagcaag tttaagatct ggaggtggca ctgagctttg gaggagcagg gggcaatgag |
| 16501 | gtggccggtg tgacgaggac tcaatgtgca agagggagag tggtggggag atgaggtgga |
| 16561 | ggggtggtcg gcggtcagat cgtggagggt ctcggacgag ggtcctgacc ctgggtctcc |
| 16621 | agtcctggga agtggagccc aggctgtacc atggctgacc tcagctcatg gcttcccctc |
| 16681 | ccacttccag cctacaggga gcccttcgag cagcacctcc tgctgggcgt cagcgtttcc |
| 16741 | tgcattgtca tcctggccgt ctgcctgttg tgctatgtca gcatcaccaa gtgagtcctg |
| 16801 | ggcccagtgc tgccgagcag tccctctgga gtgcagggtg gcagggactt gcccctctag |
| 16861 | tctgcccctt tgcagtcctc tcagtcaata atacgtattt actgagcagc tactacacac |
| 16921 | cttgagagta gagctgagaa catatcgaca aggaccccac ttttttcttt tttttttttt |
| 16981 | tttttttttt tgagacggag tctcactctg tcacccaggc tggagtatag tggcacaatc |
| 17041 | ttgcctaaca gtaacctccg cctcccgggt tcaagcaatt cttctgcctc agcctccaga |
| 17101 | gtagctggga ttacaggcgc atgccactat gcccggctaa ttttttgtat ttttggtaga |
| 17161 | gatggggttt caccatgttg gtcaggctgg tctcgaactc ctgacctcat gatctgcctg |
| 17221 | cctcagcctc ccaaagtgct gggattacag gtgtgagcca ctgcacccaa ccaggactcc |
| 17281 | acatttctaa aaccggcatc ctactgggga gactgaaaat acatatcaat cacaaacagg |
| 17341 | tggttttcca tagtgaccca ctctctgaat gcactagacc agggtggagg ccagagatct |
| 17401 | tctggggtgc tttttgcaag ggggaccagg ataaggctct ccaaggaggg aaaatttgag |
| 17461 | gggggccctg actggggaga atgagctggc cagggataag caagatggag tcatcccaca |
| 17521 | tccccttaca acactgggtg cctgggcaac tgggggcatt tgggggcatg tggtaggagc |
| 17581 | cagaggaatt tgcgacgatt gccctgatgg agtcaggaga cctgggtttg aatcctggcc |
| 17641 | ttggagcttg gtagctggcg gccgacaagt tgctgaaacc cctgagcctg gggttcctgc |
| 17701 | tttgcagagt gacagtgatg gtgagaacat atttcatcag ccagaagagg ccaaatcaca |
| 17761 | gtaaaggctg agggaggaga tgagtggcga gtggctggga ggtggtggaa ggagcctcgt |
| 17821 | ttccagagag ctcttgccag cccttggaat catggtgtct cagagcctca gtcctcccat |
| 17881 | ctctgaaatg ggactagcaa gctcaacctc actaagtcag gattagaggt ggctaaggat |
| 17941 | tattaacatg attgatgaaa gtgcccactc ttggcccagc acacactagg taggcaggga |
| 18001 | atgcaaattc ccctccatat cttgtcactg atgcctccga gcaaccttgg actgatcgcc |
| 18061 | ttgctctgag cctcagtttc cccatcacct gtacctcttc ccactcccca tcactatatc |
| 18121 | ccagcatgcc agcctctttg ctgttctttg tctttggttt cttgttttgt tctgtttttt |
| 18181 | agacagggtc tcactctgtt agccaggctg aagtgcagtg gcgcggttac ggctcactgc |
| 18241 | agcctccaat tcctgggcta aagagatcct cccatttcaa cttccagagc agctgggaca |
| 18301 | acaggcgctt gccaccacac ctggctaatt ttcttatttt aatttaattt tattttattt |
| 18361 | tttgggacag agtggagtct caaaaaccaa gctagagtgc agtggtgcga tctcgactca |
| 18421 | ctgcaatctc tgcctcccgg gttcaagcga ttctcctgcc ttagcctccc gactagctgg |
| 18481 | gattacaggc gtgtgccacg acacccagct aatttttgta tttttagtag agatggggtt |
| 18541 | tcaccatgtt ggccaggatg gtcttgaact cctgacctca agtgatccac ccacctcgtt |
| 18601 | ctcccaaggt gctgggtaca ggcatgagcc actgtgcctg gccaattttc ttacattttg |
| 18661 | tagagactgg ctgtcactta tgtagcccag gctgatcttg aacttctacc cctttatctt |
| 18721 | tattcatggc acttattacc atgaatgaat gacctcatat aagcatttct ttcgtttttt |
| 18781 | tttttttttc tttgagatgg agtctcatgt tgtcccccag gctggagtgc agtggcgcga |
| 18841 | tctcagctca ctgcaacctc cgccttccgg gttcaagcga ttctcctgcc tcagcctcct |
| 18901 | gagtagctgg gattgcaggc gcctgccacc atgcctggct aagttttgca tttttagtag |
| 18961 | agacggtgtt tcaccatatt ggccaggctg gtctcgaact tctgacctca ggtgatacac |
| 19021 | ctgccttggc ctcccaaagt gctgggatta caggcgtgag ccgccatgcc tggcctcata |
| 19081 | taagcatttc tgtctccatt tatcatccat ctttccctct tgaaggtcag tttcaccaag |
| 19141 | gcaggcatct ttgtctcgtt cactgttgtg gcctcagggc caggcacagt gagtcaaaca |
| 19201 | tagaaggtgc tcaataaata tgtgtttatt tattgaaacc atgggcagag gctaattcag |
| 19261 | aagcggtctg aggaccttac ctcccagtga tgatgcacca tggccccagg caggccagga |
| 19321 | agagagaagg gttgtgtttc tccgtaggtc ccccagcttc ccaggccatc ccaggccatt |
| 19381 | ccctggtcat ttgccctcag ctgctctgaa aaagggattg ttgaggggaa cctagaatcc |
| 19441 | tctctctgca gtttgagtct ttcctaatcc cctggggtct cattcccact gaggacatag |
| 19501 | gtggcctcct caggaactct gtgctgggta acagaatgcg ggagtgtgaa cctggctctg |
| 19561 | ccacctacca gctgtcactc cacctccttg ggcctcactc tcctcatctg tagaataggg |
| 19621 | ttagcaatag aatccatgtc accaggttag aatgatgagt cagtggtttg acctccagaa |
| 19681 | actaatcagc ctgatctctg atgccaaata agtattggtg ataacgacca cttttatggg |
| 19741 | aggagcgttc acctgtcaat aattcagaga tcaacacctt ttccttttgt ttttcaggat |
| 19801 | taagaaagaa tggtgggatc agattcccaa cccagcccgc agccgcctcg tggctataat |
| 19861 | aatccaggat gctcaggtag gagtaggcgt ggatgaggac atgtgggact gtgtacatga |
| 19921 | agaagtgtgg ttcagaacac ctgggctgtt aaggaccttc actggcttct ggaatggcaa |
| 19981 | atagacagtc aggagggttg caggggagac agaggcagaa gccgaatgag gtcattagca |
| 20041 | gaccagaggc tttcccgccc ttccccttgg caatcccagc ctggggtggg cttctctggg |
| 20101 | gttggtttcc tgtttttttc cctccccttg ggagaatgac ccttgggtca tcatcactgt |
| 20161 | gtcattccct ggggaggtgc cagtaccagg gctagaggcc agaaggagtg gaggaaggag |
| 20221 | agggtgacag gctttctgtg tcttcttctt aagcatagga aactgccccc gaagcactag |
| 20281 | caaatccctt ccgggttctc attggcctga aatgtatccc acccctaagc caggggtgga |
| 20341 | gtcagcttcc ccaaggcgat ggtcctgtgg gtgagtgggt ggggtttgcc tgagcaagat |
| 20401 | gagagttctc taggtaggag aaagggggat tataggtcct gtctagaaga gaaggtctga |
| 20461 | gggtccttgc ttttccaggg actctggaat ctagtggtgt tggctttgaa tcctgactct |
| 20521 | gccactcact ggcagtgtgg acttgagcaa gttgcttaat tctctgagcc tcagtttcct |
| 20581 | cttgtgggtt ataacagtgt ttacctggta ggacagatat tggaatttat tgagacaata |
| 20641 | catataaagt gcatattcca gcctcttgca aataccaagt gccatttatg tatcagttag |
| 20701 | tgtttgctgt gtaacaaatg accccgaaat gtagagggtt acaacaactt tatttagctt |
| 20761 | atgcttctgc aggctggcat ttggggctgg gctcagcagt gagggtggcg ggggaggctg |
| 20821 | ggctgggctg ggctgggcag atctgaattg agctgacccg tccccgtagc ctccctccgt |
| 20881 | gtctgacagt tggctttttt tttttttttc tttttctgag acggagtttt gctcttattg |
| 20941 | cccaggagtg caatggcgtg atcttggctc actgcaacct ctgcctcctg ggttcaagca |
| 21001 | attttcttgc ctcagcctcc caagtagctg ggattacagg catgtgccac cacgccaggc |
| 21061 | taattttgta tttttaatag agatggggtt tcttcatgtt ggtcaggctg gtctggaact |
| 21121 | cctaatatca gatgatccac ccacctcagc ctcccaaagt gctgggatta caggcgtgag |
| 21181 | ccactgcacc cagcctagtt ggctgacttt tacctgggac agtgcaggtg cctgagccat |
| 21241 | gtgcctctca ctctccagca ggccggccca ggcttgttta cagagtggct cagttttcaa |
| 21301 | gggtgggaag tcccaaggct tcttgaggcc taggcgcagc actggcatga tatcacttcc |
| 21361 | atcacattct atgggcccaa gcaagtccca gggccagtgt agattcaagg gatgggagga |
| 21421 | gattcagagc actcctctgt ggccactttt gccatcgacc acagtccctg taaatattag |
| 21481 | gacaatgtaa ttaattccca ggaatctgag gctcagaaag cgtaagtgac ctgttggact |
| 21541 | tctgatctgt gtgatgtcga ggcttgtacc ccttcctgag cattgccgta ctccaggccg |
| 21601 | ggctgcaagg ccactctgct ctttcattgg ctgtctctgt attttagggg tcacagtggg |
| 21661 | agaagcggtc ccgaggccag gaaccagcca agtgcccgta tgtatctgaa cttaggtcac |
| 21721 | agcctgcatg cattgggaag gtgatagaat tggagaggca agcccctagc tccatgtctg |
| 21781 | ccttctcttc cctgcattcg gtaattgccc tgtgacatta gccttcaagg gacggcagga |
| 21841 | ggaggggtgt tctggaaacg tggactgctg gccaagcccc ctgagtttca ctggtgtgtc |
| 21901 | aggtacatgg tgatacccct tgggagtgct gttatagtta acaaccagag cagccgtgcc |
| 21961 | tgttgttaaa atcttgacct aattgtatac ttgtcggcaa atagccacta tcctgaacac |
| 22021 | tcccctcctt ttfrttaata tacaggatct cactctgtgg cccaggctgg tgtgcagtgg |
| 22081 | tgcgatcata gctcactgca ccttcaaact cctgagctca agtgatcctc ccatcttagc |
| 22141 | ctcccgagta gctgatacta cagatgtgca ttaccacgcc tggctatttt aaaaggtttt |
| 22201 | tgcctgtaat tccagctact caggaggctg aggcatgaga atcacttgaa cccgggaggc |
| 22261 | agaggttgca gtgagcgcag attgtgccac tgcactccag cctgggcgac agagtgagac |
| 22321 | tcttgtctca aaaaaaataa taccaaaaaa agtttttgta aagacaagct ctcgctgtgt |
| 22381 | tgccccgcca ctgtggcctc cttagcttct tccctggggc ctgctggacc tttccatact |
| 22441 | ccagaaacta aagggggtcc aggaccctgc ttcaacccta ggatcccgca tctttttttt |
| 22501 | tttttttttt ttggacgcag ggtcttgctg tgtccctcag gctggagtgc agtgattcac |
| 22561 | tgcagcctca aactcgtggg ctcaagtgat tctctagcct cagccttcta agtagctggg |
| 22621 | actacagtca tacaccaaca tgcccagcta attttccttt tttttaattc ttgtagagat |
| 22681 | gtttgagacg gcttgggctc tgttgcccag gctgttctca aactcctgag ctcaagcgat |
| 22741 | cctccctcct cagcctccta aagtgctggg attacaggcg tgagccaccg cacccggctt |
| 22801 | ccatatcctt tctaattggt catggcttgg gataatggtg ttgcttttaa ttatcatcat |
| 22861 | ccataaagac tttttcttac tcaacagatc tgagcttgta tttggtgccc aggacatgtg |
| 22921 | ctgggttccc gaaatcccaa agacacagac cctaccctca gggatttctc attctagcaa |
| 22981 | catagactga tcaattactg attataacgt tagaaggcat gtctgaagta gacagccatc |
| 23041 | aggacatggt gatttcaggc tgggctttga agaatgaata ggagtttttc aagtgtcgaa |
| 23101 | actgaaccct gaccaacctt tgcttttgca gacactggaa gaattgtctt accaagctct |
| 23161 | tgccctgttt tctggagcac aacatgaaaa gggatgaaga tcctcacaag gctgccaaag |
| 23221 | agatgccttt ccagggctct ggaaaatcag catggtgccc agtggagatc agcaagacag |
| 23281 | tcctctggcc agagagcatc agcgtggtgc gatgtgtgga gttgtttgag gccccggtgg |
| 23341 | agtgtgagga ggaggaggag gtagaggaag aaaaagggag cttctgtgca tcgcctgaga |
| 23401 | gcagcaggga tgacttccag gagggaaggg agggcattgt ggcccggcta acagagagcc |
| 23461 | tgttcctgga cctgctcgga gaggagaatg ggggcttttg ccagcaggac atgggggagt |
| 23521 | catgccttct tccaccttcg ggaagtacga gtgctcacat gccctgggat gagttcccaa |
| 23581 | gtgcagggcc caaggaggca cctccctggg gcaaggagca gcctctccac ctggagccaa |
| 23641 | gtcctcctgc cagcccgacc cagagtccag acaacctgac ttgcacagag acgcccctcg |
| 23701 | tcatcgcagg caaccctgct taccgcagct tcagcaactc cctgagccag tcaccgtgtc |
| 23761 | ccagagagct gggtccagac ccactgctgg ccagacacct ggaggaagta gaacccgaga |
| 23821 | tgccctgtgt cccccagctc tctgagccaa ccactgtgcc ccaacctgag ccagaaacct |
| 23881 | gggagcagat cctccgccga aatgtcctcc agcatggggc agctgcagcc cccgtctcgg |
| 23941 | cccccaccag tggctatcag gagtttgtac atgcggtgga gcagggtggc acccaggcca |
| 24001 | gtgcggtggt gggcttgggt cccccaggag aggctggtta caaggccttc tcaagcctgc |
| 24061 | ttgccagcag tgctgtgtcc ccagagaaat gtgggtttgg ggctagcagt ggggaagagg |
| 24121 | ggtataagcc tttccaagac ctcattcctg gctgccctgg ggaccctgcc ccagtccctg |
| 24181 | tccccttgtt cacctttgga ctggacaggg agccacctcg cagtccgcag agctcacatc |
| 24241 | tcccaagcag ctccccagag cacctgggtc tggagccggg ggaaaaggta gaggacatgc |
| 24301 | caaagccccc acttccccag gagcaggcca cagaccccct tgtggacagc ctgggcagtg |
| 24361 | gcattgtcta ctcagccctt acctgccacc tgtgcggcca cctgaaacag tgtcatggcc |
| 24421 | aggaggatgg tggccagacc cctgtcatgg ccagtccttg ctgtggctgc tgctgtggag |
| 24481 | acaggtcctc gccccctaca acccccctga gggccccaga cccctctcca ggtggggttc |
| 24541 | cactggaggc cagtctgtgt ccggcctccc tggcaccctc gggcatctca gagaagagta |
| 24601 | aatcctcatc atccttccat cctgcccctg gcaatgctca gagctcaagc cagaccccca |
| 24661 | aaatcgtgaa ctttgtctcc gtgggaccca catacatgag ggtctcttag gtgcatgtcc |
| 24721 | tcttgttgct gagtctgcag atgaggacta gggcttatcc atgcctggga aatgccacct |
| 24781 | cctggaaggc agccaggctg gcagatttcc aaaagacttg aagaaccatg gtatgaaggt |
| 24841 | gattggcccc actgacgttg gcctaacact gggctgcaga gactggaccc cgcccagcat |
| 24901 | tgggctgggc tcgccacatc ccatgagagt agagggcact gggtcgccgt gccccacggc |
| 24961 | aggcccctgc aggaaaactg aggcccttgg gcacctcgac ttgtgaacga gttgttggct |
| 25021 | gctccctcca cagcttctgc agcagactgt ccctgttgta actgcccaag gcatgttttg |
| 25081 | cccaccagat catggcccac gtggaggccc acctgcctct gtctcactga actagaagcc |
| 25141 | gagcctagaa actaacacag ccatcaaggg aatgacttgg gcggccttgg gaaatcgatg |
| 25201 | agaaattgaa cttcagggag ggtggtcatt gcctagaggt gctcattcat ttaacagagc |
| 25261 | ttccttaggt tgatgctgga ggcagaatcc cggctgtcaa ggggtgttca gttaagggga |
| 25321 | gcaacagagg acatgaaaaa ttgctatgac taaagcaggg acaatttgct gccaaacacc |
| 25381 | catgcccagc tgtatggctg ggggctcctc gtatgcatgg aacccccaga ataaatatgc |
| 25441 | tcagccaccc tgtgggccgg gcaatccaga cagcaggcat aaggcaccag ttaccctgca |
| 25501 | tgttggccca gacctcaggt gctagggaag gcgggaacct tgggttgagt aatgctcgtc |
| 25561 | tgtgtgtttt agtttcatca cctgttatct gtgtttgctg aggagagtgg aacagaaggg |
| 25621 | gtggagtttt gtataaataa agtttctttg tctctttatt ttttatgtat taaccaaaca |
| 25681 | tacctccaga cactgctgtg agtgctgtgt ctctgttaac tcctggaatt cacccatcca |
| 25741 | gaggaaccag gatgcaagag gttaagaaac ttgccatctg ggtttgggtt ccccatacaa |
| 25801 | ggattcaaat agttgattta ggaagtaatc ccgggaaacc ctgctaaggt agtggggaac |
| 25861 | tgaggcaggg aaggacacaa accaagaaag tgttacctga aaggggtcca gatgcagacc |
| 25921 | ccaaaagagg gttcttgaat ctcatgcaag aaagaattca gagcgagtcc atagagtcag |
| 25981 | tgaaagcaag ttaatgagga aagtaaagga ataaaagaat ggctactccg tagacagagc |
| 26041 | agccctgagg gttgctggct gcctattttt atggttattg attaattata ttccaaacaa |
| 26101 | ggggtggatt attatgcctc ccttttagac catatagggt aacttcctga tgttgccatg |
| 26161 | gcatttgtaa actgtcatgg cgctgttggg agtgtagcag tgaggacaac cagaggtcac |
| 26221 | tcttgttgcc atcttggttt tggtgggtta gagccatctt ctttactgca acctgtttta |
| 26281 | tcagcaaggt ctttatgact tgtatcggtg acgacctcct gtctcattct atgactaaga |
| 26341 | atgccctaac ctcccaggaa tgcagcccag taagtctcag cctcatttta cccagcccct |
| 26401 | cttcaaagct ccagtttaaa taaacctctg acaaaagggt gagttattca acagattacc |
| 26461 | agcatgagca actgatgctt acctgccggg gatctctgga agaccatgca tggcacatgc |
| 26521 | ccagttatgc ctgcaaagga gagggagctg |

SEQ ID NO: 3 is also illustrative for an IL4R coding sequence which is homologous to the wild-type IL4R sequence shown in SEQ ID NO: 1 and which comprises additional identified variations to the wild-type sequence. Said SEQ ID NO: 3 is accessible under Accession number AF421855 (gi: 15987825) of the NCBI database.

Variations to the wild-type sequence of IL4R are deducible from this NCBI entry and are also illustrated by the following information. These variations may also be comprised (and in addition) be detected in the methods provided herein. Accordingly, the exemplified variations provided below represent some naturally occurring variants or isoforms of human IL4R. The means, methods and uses provided in this invention are also related to the determination of the I50V/I75V variant of IL4R in these isoforms/variants.

Such isoforms/variants/variations comprise the following (in respect to SEQ ID No. 3 provided above):

| | |
|---|---|
| repeat region | 1216 . . . 1313 |
| | /rpt_family = “L2” |
| | /rpt_type = dispersed |
| repeat region | 1314 . . . 1606 |
| | /rpt_family = “AluJo” |
| | /rpt_type = dispersed |
| variation | 1454 |
| | /gene = “IL4R” |
| | /frequency = “0.01” |
| | /replace = “a” |
| variation | 1596 |
| | /gene = “IL4R” |
| | /frequency = “0.01” |
| | /replace = “c” |
| repeat region | 1607 . . . 2263 |
| | /rpt_family = “L2” |
| | /rpt_type = dispersed |
| variation | 1673 |
| | /gene = “IL4R” |
| | /frequency = “0.02” |
| | /replace = “c” |
| variation | 2265 |
| | /gene = “IL4R” |
| | /frequency = “0.12” |
| | /replace = “t” |
| variation | 2371 |
| | /gene = “IL4R” |
| | /frequency = “0.14” |
| | /replace = “a” |
| repeat region | 2404 . . . 2593 |
| | /rpt_family = “LTR33” |
| | /rpt_type = dispersed |
| variation | 2438 |
| | /gene = “IL4R” |
| | /frequency = “0.02” |
| | /replace = “t” |
| variation | 2487 |
| | /gene = “IL4R” |
| | /frequency = “0.02” |
| | /replace = “g” |
| variation | 2490 |
| | /gene = “IL4R” |
| | /frequency = “0.02” |
| | /replace = “a” |
| variation | 2517 |
| | /gene = “IL4R” |
| | /frequency = “0.02” |
| | /replace = “c” |
| variation | 2740 |
| | /gene = “IL4R” |
| | /frequency = “0.01” |
| | /replace = “a” |
| variation | 2782 |
| | /gene = “IL4R” |
| | /frequency = “0.17” |
| | /replace = “a” |
| variation | 2820 |
| | /gene = “IL4R” |
| | /frequency = “0.02” |
| | /replace = “a” |
| variation | 2909 |
| | /gene = “IL4R” |
| | /frequency = “0.13” |
| | /replace = “t” |
| variation | 3337 |
| | /gene = “IL4R” |
| | /frequency = “0.03” |
| | /replace = “a” |
| repeat region | 3373 . . . 3536 |
| | /rpt_family = “Charlie1” |
| | /rpt_type = dispersed |
| repeat region | 3537 . . . 3666 |
| | /rpt_family = “FLAM_C” |
| | /rpt_type = dispersed |
| variation | 3619 |
| | /gene = “IL4R” |
| | /frequency = “0.15” |
| | /replace = “a” |
| repeat region | 3677 . . . 3847 |
| | /rpt_family = “Charlie1” |
| | /rpt_type = dispersed |
| repeat region | 3849 . . . 3870 |
| | /rpt_family = “MER5A” |
| | /rpt_type = dispersed |
| variation | 3913 |
| | /gene = “IL4R” |
| | /frequency = “0.17” |
| | /replace = “t” |
| variation | 4083 |
| | /gene = “IL4R” |
| | /frequency = “0.44” |
| | /replace = “c” |
| repeat region | 4158 . . . 4236 |
| | /rpt_family = “MER58A” |
| | /rpt_type = dispersed |
| repeat region | 4280 . . . 4604 |
| | /rpt_family = “AluSq” |
| | /rpt_type = dispersed |
| variation | 4395 |
| | /gene = “IL4R” |
| | /frequency = “0.04” |
| | /replace = “g” |
| variation | 4503 |
| | /gene = “IL4R” |
| | /frequency = “0.16” |
| | /replace = “c” |
| variation | 4557 |
| | /gene = “IL4R” |
| | /frequency = “0.01” |
| | /replace = “g” |
| variation | 4560 |
| | /gene = “IL4R” |
| | /frequency = “0.47” |
| | /replace = “g” |
| variation | 4606 |
| | /gene = “IL4R” |
| | /frequency = “0.01” |
| | /replace = “t” |
| repeat region | 4609 . . . 4896 |
| | /rpt_family = “L1PB4” |
| | /rpt_type = dispersed |
| variation | 4841 |
| | /gene = “IL4R” |
| | /frequency = “0.16” |
| | /replace = “t” |
| repeat region | 4897 . . . 5209 |
| | /rpt_family = “AluSx” |
| | /rpt_type = dispersed |
| variation | 4905 |
| | /gene = “IL4R” |
| | /frequency = “0.19” |
| | /replace = “g” |
| variation | 4918 |
| | /gene = “IL4R” |
| | /frequency = “0.34” |
| | /replace = “a” |
| variation | 4986 |
| | /gene = “IL4R” |
| | /frequency = “0.02” |
| | /replace = “a” |
| misc feature | 5165 . . . 5307 |
| | /gene = “IL4R” |
| | /note = “Region not scanned for variation” |
| repeat region | 5210 . . . 5232 |
| | /rpt_family = “L1PB4” |
| | /rpt_type = dispersed |
| repeat region | 5234 . . . 5331 |
| | /rpt_family = “MER58B” |
| | /rpt_type = dispersed |
| variation | 5758 |
| | /gene = “IL4R” |
| | /frequency = “0.48” |
| | /replace = “a” |
| variation | 5914 |
| | /gene = “IL4R” |
| | /frequency = “0.35” |
| | /replace = “t” |
| variation | 5953 |
| | /gene = “IL4R” |
| | /frequency = “0.38” |
| | /replace = “t” |
| variation | 6162 |
| | /gene = “IL4R” |
| | /frequency = “0.02” |
| | /replace = “g” |
| variation | 6222 |
| | /gene = “IL4R” |
| | /frequency = “0.42” |
| | /replace = “g” |
| variation | 6235 |
| | /gene = “IL4R” |
| | /frequency = “0.01” |
| | /replace = “t” |
| variation | 6235 |
| | /gene = “IL4R” |
| | /frequency = “0.02” |
| | /replace = “a” |
| variation | 6244 |
| | /gene = “IL4R” |
| | /frequency = “0.02” |
| | /replace = “c” |
| variation | 6296 |
| | /gene = “IL4R” |
| | /frequency = “0.01” |
| | /replace = “t” |
| variation | 6447 |
| | /gene = “IL4R” |
| | /frequency = “0.08” |
| | /replace = “a” |
| repeat region | 6565 . . . 6876 |
| | /rpt_family = “AluSx” |
| | /rpt_type = dispersed |
| variation | 6568 |
| | /gene = “IL4R” |
| | /frequency = “0.02” |
| | /replace = “t” |
| variation | 6619 |
| | /gene = “IL4R” |
| | /frequency = “0.02” |
| | /replace = “g” |
| variation | 6663 |
| | /gene = “IL4R” |
| | /frequency = “0.02” |
| | /replace = “g” |
| variation | 6834 |
| | /gene = “IL4R” |
| | /frequency = “0.02” |
| | /replace = “c” |
| repeat region | 6955 . . . 7117 |
| | /rpt_family = “AluJb” |
| | /rpt_type = dispersed |
| variation | 7086 |
| | /gene = “IL4R” |
| | /frequency = “0.01” |
| | /replace = “a” |
| variation | 7339 |
| | /gene = “IL4R” |
| | /frequency = “0.05” |
| | /replace = “a” |
| variation | 7482 |
| | /gene = “IL4R” |
| | /frequency = “0.06” |
| | /replace = “t” |
| variation | 7595 |
| | /gene = “IL4R” |
| | /frequency = “0.01” |
| | /replace = “t” |
| variation | 7613 |
| | /gene = “IL4R” |
| | /frequency = “0.01” |
| | /replace = “t” |
| variation | 7653 |
| | /gene = “IL4R” |
| | /frequency = “0.04” |
| | /replace = “a” |
| variation | 7685 |
| | /gene = “IL4R” |
| | /frequency = “0.01” |
| | /replace = “g” |
| variation | 7687 |
| | /gene = “IL4R” |
| | /frequency = “0.37” |
| | /replace = “t” |
| variation | 7745 |
| | /gene = “IL4R” |
| | /frequency = “0.08” |
| | /replace = “a” |
| variation | 7758 |
| | /gene = “IL4R” |
| | /frequency = “0.50” |
| | /replace = “g” |
| variation | 7906 |
| | /gene = “IL4R” |
| | /frequency = “0.05” |
| | /replace = “a” |
| repeat region | 8065 . . . 8366 |
| | /rpt_family = “AluSx” |
| | /rpt_type = dispersed |
| variation | 8199 |
| | /gene = “IL4R” |
| | /frequency = “0.02” |
| | /replace = “t” |
| variation | 8293 |
| | /gene = “IL4R” |
| | /frequency = “0.01” |
| | /replace = “t” |
| variation | 8318 |
| | /gene = “IL4R” |
| | /frequency = “0.42” |
| | /replace = “t” |
| variation | 8414 |
| | /gene = “IL4R” |
| | /frequency = “0.01” |
| | /replace = “a” |
| variation | 8443 |
| | /gene = “IL4R” |
| | /frequency = “0.14” |
| | /replace = “a” |
| variation | 8503 |
| | /gene = “IL4R” |
| | /frequency = “0.35” |
| | /replace = “a” |
| variation | 8576 |
| | /gene = “IL4R” |
| | /frequency = “0.48” |
| | /replace = “a” |
| repeat region | 8595 . . . 8737 |
| | /rpt_family = “MIR” |
| | /rpt_type = dispersed |
| variation | 8616 |
| | /gene = “IL4R” |
| | /frequency = “0.49” |
| | /replace = “a” |
| variation | 8749 |
| | /gene = “IL4R” |
| | /frequency = “0.07” |
| | /replace = “t” |
| variation | 8851 |
| | /gene = “IL4R” |
| | /frequency = “0.08” |
| | /replace = “a” |
| variation | 9130 |
| | /gene = “IL4R” |
| | /frequency = “0.01” |
| | /replace = “a” |
| repeat region | 9322 . . . 9751 |
| | /rpt_family = “MLT1K” |
| | /rpt_type = dispersed |
| variation | 9399 |
| | /gene = “IL4R” |
| | /frequency = “0.49” |
| | /replace = “a” |
| variation | 9596 |
| | /gene = “IL4R” |
| | /frequency = “0.10” |
| | /replace = “a” |
| variation | 9640 |
| | /gene = “IL4R” |
| | /frequency = “0.01” |
| | /replace = “c” |
| variation | 9787 |
| | /gene = “IL4R” |
| | /frequency = “0.05” |
| | /replace = “” |
| repeat region | 9990 . . . 10091 |
| | /rpt_family = “MLT1L” |
| | /rpt_type = dispersed |
| variation | 10182 |
| | /gene = “IL4R” |
| | /frequency = “0.47” |
| | /replace = “a” |
| variation | 10183 |
| | /gene = “IL4R” |
| | /frequency = “0.47” |
| | /replace = “t” |
| repeat region | 10296 . . . 10450 |
| | /rpt_family = “L2” |
| | /rpt_type = dispersed |
| variation | 10411 |
| | /gene = “IL4R” |
| | /frequency = “0.04” |
| | /replace = “t” |
| variation | 10487 |
| | /gene = “IL4R” |
| | /frequency = “0.05” |
| | /replace = “a” |
| repeat region | 10583 . . . 10619 |
| | /rpt_family = “MIR” |
| | /rpt_type = dispersed |
| variation | 10607 |
| | /gene = “IL4R” |
| | /frequency = “0.04” |
| | /replace = “g” |
| repeat region | 10620 . . . 10929 |
| | /rpt_family = “AluJb” |
| | /rpt_type = dispersed |
| variation | 10644 . . . 10646 |
| | /gene = “IL4R” |
| | /frequency = “0.96” |
| | /replace = “” |
| variation | 10725 |
| | /gene = “IL4R” |
| | /frequency = “0.43” |
| | /replace = “c” |
| variation | 10871 |
| | /gene = “IL4R” |
| | /frequency = “0.05” |
| | /replace = “a” |
| variation | 10895 |
| | /gene = “IL4R” |
| | /frequency = “0.02” |
| | /replace = “a” |
| variation | 10920 |
| | /gene = “IL4R” |
| | /frequency = “0.09” |
| | /replace = “a” |
| repeat region | 10930 . . . 10978 |
| | /rpt_family = “MIR” |
| | /rpt_type = dispersed |
| variation | 11135 |
| | /gene = “IL4R” |
| | /frequency = “0.02” |
| | /replace = “g” |
| variation | 11351 |
| | /gene = “IL4R” |
| | /frequency = “0.08” |
| | /replace = “t” |
| repeat region | 11819 . . . 12359 |
| | /rpt_family = “MLT1E” |
| | /rpt_type = dispersed |
| variation | 12007 |
| | /gene = “IL4R” |
| | /frequency = “0.02” |
| | /replace = “g” |
| variation | 12091 |
| | /gene = “IL4R” |
| | /frequency = “0.06” |
| | /replace = “c” |
| repeat region | 12380 . . . 12692 |
| | /rpt_family = “AluSx” |
| | /rpt_type = dispersed |
| variation | 12408 |
| | /gene = “IL4R” |
| | /frequency = “0.43” |
| | /replace = “t” |
| variation | 12598 |
| | /gene = “IL4R” |
| | /frequency = “0.06” |
| | /replace = “a” |
| variation | 12604 |
| | /gene = “IL4R” |
| | /frequency = “0.06” |
| | /replace = “a” |
| repeat region | 12729 . . . 12811 |
| | /rpt_family = “L1MC4a” |
| | /rpt_type = dispersed |
| variation | 12787 |
| | /gene = “IL4R” |
| | /frequency = “0.04” |
| | /replace = “c” |
| variation | 12822 |
| | /gene = “IL4R” |
| | /frequency = “0.02” |
| | /replace = “c” |
| repeat region | 12941 . . . 13228 |
| | /rpt_family = “MIR” |
| | /rpt_type = dispersed |
| variation | 12951 |
| | /gene = “IL4R” |
| | /frequency = “0.01” |
| | /replace = “a” |
| variation | 13060 |
| | /gene = “IL4R” |
| | /frequency = “0.01” |
| | /replace = “c” |
| variation | 13168 |
| | /gene = “IL4R” |
| | /frequency = “0.06” |
| | /replace = “a” |
| variation | 13243 |
| | /gene = “IL4R” |
| | /frequency = “0.13” |
| | /replace = “g” |
| variation | 13291 |
| | /gene = “IL4R” |
| | /frequency = “0.02” |
| | /replace = “t” |
| variation | 13715 |
| | /gene = “IL4R” |
| | /frequency = “0.33” |
| | /replace = “t” |
| repeat region | 13720 . . . 13915 |
| | /rpt_family = “MLT1J2” |
| | /rpt_type = dispersed |
| variation | 13757 |
| | /gene = “IL4R” |
| | /frequency = “0.29” |
| | /replace = “a” |
| variation | 13790 |
| | /gene = “IL4R” |
| | /frequency = “0.24” |
| | /replace = “g” |
| variation | 13810 |
| | /gene = “IL4R” |
| | /frequency = “0.33” |
| | /replace = “t” |
| variation | 13902 |
| | /gene = “IL4R” |
| | /frequency = “0.23” |
| | /replace = “g” |
| variation | 13912 |
| | /gene = “IL4R” |
| | /frequency = “0.02” |
| | /replace = “c” |
| repeat region | 13916 . . . 14009 |
| | /rpt_family = “Charlie4a” |
| | /rpt_type = dispersed |
| variation | 13995 |
| | /gene = “IL4R” |
| | /frequency = “0.01” |
| | /replace = “a” |
| repeat region | 14011 . . . 14243 |
| | /rpt_family = “AluJb” |
| | /rpt_type = dispersed |
| variation | 14029 |
| | /gene = “IL4R” |
| | /frequency = “0.03” |
| | /replace = “a” |
| variation | 14129 |
| | /gene = “IL4R” |
| | /frequency = “0.03” |
| | /replace = “g” |
| variation | 14239 . . . 14243 |
| | /gene = “IL4R” |
| | /frequency = “0.95” |
| | /replace = “” |
| repeat region | 14244 . . . 14516 |
| | /rpt_family = “Charlie4a” |
| | /rpt_type = dispersed |
| variation | 14368 |
| | /gene = “IL4R” |
| | /frequency = “0.36” |
| | /replace = “a” |
| variation | 14480 |
| | /gene = “IL4R” |
| | /frequency = “0.13” |
| | /replace = “g” |
| repeat region | 14564 . . . 14680 |
| | /rpt_family = “AluJb” |
| | /rpt_type = dispersed |
| variation | 14639 |
| | /gene = “IL4R” |
| | /frequency = “0.03” |
| | /replace = “t” |
| variation | 14680 |
| | /gene = “IL4R” |
| | /frequency = “0.04” |
| | /replace = “t” |
| repeat region | 14686 . . . 14827 |
| | /rpt_family = “Charlie4a” |
| | /rpt_type = dispersed |
| repeat region | 14828 . . . 14998 |
| | /rpt_family = “MLT1J” |
| | /rpt_type = dispersed |
| variation | 15015 |
| | /gene = “IL4R” |
| | /frequency = “0.36” |
| | /replace = “g” |
| variation | 15030 |
| | /gene = “IL4R” |
| | /frequency = “0.05” |
| | /replace = “g” |
| repeat region | 15069 . . . 15435 |
| | /rpt_family = “MLT1L” |
| | /rpt_type = dispersed |
| variation | 15176 |
| | /gene = “IL4R” |
| | /frequency = “0.32” |
| | /replace = “g” |
| variation | 15234 |
| | /gene = “IL4R” |
| | /frequency = “0.01” |
| | /replace = “a” |
| variation | 15242 |
| | /gene = “IL4R” |
| | /frequency = “0.01” |
| | /replace = “t” |
| variation | 15244 |
| | /gene = “IL4R” |
| | /frequency = “0.01” |
| | /replace = “a” |
| variation | 15389 |
| | /gene = “IL4R” |
| | /frequency = “0.01” |
| | /replace = “a” |
| repeat region | 15545 . . . 15604 |
| | /rpt_family = “MIR” |
| | /rpt_type = dispersed |
| variation | 15592 |
| | /gene = “IL4R” |
| | /frequency = “0.12” |
| | /replace = “a” |
| variation | 15688 |
| | /gene = “IL4R” |
| | /frequency = “0.05” |
| | /replace = “g” |
| variation | 15708 |
| | /gene = “IL4R” |
| | /frequency = “0.01” |
| | /replace = “a” |
| repeat region | 15719 . . . 15819 |
| | /rpt_family = “L2” |
| | /rpt_type = dispersed |
| variation | 15858 |
| | /gene = “IL4R” |
| | /frequency = “0.08” |
| | /replace = “c” |
| repeat region | 15889 . . . 16212 |
| | /rpt_family = “L2” |
| | /rpt_type = dispersed |
| variation | 15960 |
| | /gene = “IL4R” |
| | /frequency = “0.01” |
| | /replace = “a” |
| variation | 16061 |
| | /gene = “IL4R” |
| | /frequency = “0.05” |
| | /replace = “g” |
| variation | 16124 |
| | /gene = “IL4R” |
| | /frequency = “0.26” |
| | /replace = “g” |
| repeat region | 16220 . . . 16386 |
| | /rpt_family = “MIR” |
| | /rpt_type = dispersed |
| repeat region | 16428 . . . 16594 |
| | /rpt_family = “L2” |
| | /rpt_type = dispersed |
| variation | 16431 |
| | /gene = “IL4R” |
| | /frequency = “0.10” |
| | /replace = “t” |
| variation | 16544 |
| | /gene = “IL4R” |
| | /frequency = “0.05” |
| | /replace = “c” |
| variation | 16656 |
| | /gene = “IL4R” |
| | /frequency = “0.01” |
| | /replace = “g” |
| variation | 16788 |
| | /gene = “IL4R” |
| | /frequency = “0.03” |
| | /replace = “g” |
| misc feature | 16817 . . . 16969 |
| | /gene = “IL4R” |
| | /note = “Region not scanned for variation” |
| repeat region | 16962 . . . 17272 |
| | /rpt_family = “AluSg” |
| | /rpt_type = dispersed |
| variation | 17088 |
| | /gene = “IL4R” |
| | /frequency = “0.02” |
| | /replace = “t” |
| variation | 17134 |
| | /gene = “IL4R” |
| | /frequency = “0.01” |
| | /replace = “t” |
| variation | 17135 |
| | /gene = “IL4R” |
| | /frequency = “0.01” |
| | /replace = “a” |
| variation | 17296 |
| | /gene = “IL4R” |
| | /frequency = “0.07” |
| | /replace = “c” |
| variation | 17298 |
| | /gene = “IL4R” |
| | /frequency = “0.08” |
| | /replace = “” |
| variation | 17387 |
| | /gene = “IL4R” |
| | /frequency = “0.26” |
| | /replace = “c” |
| variation | 17399 |
| | /gene = “IL4R” |
| | /frequency = “0.01” |
| | /replace = “g” |
| repeat region | 17404 . . . 17492 |
| | /rpt_family = “L2” |
| | /rpt_type = dispersed |
| variation | 17424 |
| | /gene = “IL4R” |
| | /frequency = “0.02” |
| | /replace = “c” |
| variation | 17468 |
| | /gene = “IL4R” |
| | /frequency = “0.01” |
| | /replace = “t” |
| variation | 17533 |
| | /gene = “IL4R” |
| | /frequency = “0.26” |
| | /replace = “g” |
| variation | 17560 |
| | /gene = “IL4R” |
| | /frequency = “0.17” |
| | /replace = “c” |
| repeat region | 17608 . . . 17737 |
| | /rpt_family = “MIR” |
| | /rpt_type = dispersed |
| variation | 17659 |
| | /gene = “IL4R” |
| | /frequency = “0.02” |
| | /replace = “t” |
| repeat region | 17978 . . . 18086 |
| | /rpt_family = “MIR” |
| | /rpt_type = dispersed |
| repeat region | 18158 . . . 18329 |
| | /rpt_family = “AluJo” |
| | /rpt_type = dispersed |
| variation | 18306 |
| | /gene = “IL4R” |
| | /frequency = “0.02” |
| | /replace = “t” |
| repeat region | 18330 . . . 18643 |
| | /rpt_family = “AluSx” |
| | /rpt_type = dispersed |
| variation | 18351 |
| | /gene = “IL4R” |
| | /frequency = “0.35” |
| | /replace = “a” |
| variation | 18394 |
| | /gene = “IL4R” |
| | /frequency = “0.36” |
| | /replace = “g” |
| repeat region | 18644 . . . 18707 |
| | /rpt_family = “AluJo” |
| | /rpt_type = dispersed |
| variation | 18665 . . . 18666 |
| | /gene = “IL4R” |
| | /frequency = “0.11” |
| | /replace = “” |
| repeat region | 18709 . . . 18765 |
| | /rpt_family = “L2” |
| | /rpt_type = dispersed |
| repeat region | 18766 . . . 19075 |
| | /rpt_family = “AluSx” |
| | /rpt_type = dispersed |
| variation | 19053 |
| | /gene = “IL4R” |
| | /frequency = “0.19” |
| | /replace = “a” |
| variation | 19056 |
| | /gene = “IL4R” |
| | /frequency = “0.01” |
| | /replace = “a” |
| variation | 19063 |
| | /gene = “IL4R” |
| | /frequency = “0.31” |
| | /replace = “a” |
| repeat region | 19076 . . . 19237 |
| | /rpt_family = “L2” |
| | /rpt_type = dispersed |
| variation | 19170 |
| | /gene = “IL4R” |
| | /frequency = “0.17” |
| | /replace = “a” |
| variation | 19300 |
| | /gene = “IL4R” |
| | /frequency = “0.01” |
| | /replace = “g” |
| repeat region | 19532 . . . 19671 |
| | /rpt_family = “MIR” |
| | /rpt_type = dispersed |
| variation | 20015 |
| | /gene = “IL4R” |
| | /frequency = “0.17” |
| | /replace = “a” |
| repeat region | 20210 . . . 20332 |
| | /rpt_family = “MLT1L” |
| | /rpt_type = dispersed |
| misc feature | 20330 . . . 20467 |
| | /gene = “IL4R” |
| | /note = “Region not scanned for variation” |
| repeat region | 20476 . . . 20682 |
| | /rpt_family = “MIR” |
| | /rpt_type = dispersed |
| variation | 20497 . . . 20499 |
| | /gene = “IL4R” |
| | /frequency = “0.63” |
| | /replace = “” |
| repeat region | 20689 . . . 20815 |
| | /rpt_family = “MLT1H” |
| | /rpt_type = dispersed |
| variation | 20719 |
| | /gene = “IL4R” |
| | /frequency = “0.36” |
| | /replace = “c” |
| variation | 20809 |
| | /gene = “IL4R” |
| | /frequency = “0.03” |
| | /replace = “t” |
| repeat region | 20839 . . . 20894 |
| | /rpt_family = “MLT1H” |
| | /rpt_type = dispersed |
| repeat region | 20895 . . . 21195 |
| | /rpt_family = “AluSp” |
| | /rpt_type = dispersed |
| variation | 20958 |
| | /gene = “IL4R” |
| | /frequency = “0.31” |
| | /replace = “a” |
| variation | 20981 |
| | /gene = “IL4R” |
| | /frequency = “0.02” |
| | /replace = “g” |
| variation | 20985 |
| | /gene = “IL4R” |
| | /frequency = “0.18” |
| | /replace = “t” |
| variation | 21100 |
| | /gene = “IL4R” |
| | /frequency = “0.32” |
| | /replace = “c” |
| variation | 21132 |
| | /gene = “IL4R” |
| | /frequency = “0.39” |
| | /replace = “g” |
| variation | 21144 |
| | /gene = “IL4R” |
| | /frequency = “0.01” |
| | /replace = “t” |
| variation | 21145 |
| | /gene = “IL4R” |
| | /frequency = “0.31” |
| | /replace = “t” |
| repeat region | 21196 . . . 21438 |
| | /rpt_family = “MLT1H” |
| | /rpt_type = dispersed |
| variation | 21510 |
| | /gene = “IL4R” |
| | /frequency = “0.24” |
| | /replace = “a” |
| variation | 21877 |
| | /gene = “IL4R” |
| | /frequency = “0.02” |
| | /replace = “a” |
| variation | 21956 |
| | /gene = “IL4R” |
| | /frequency = “0.05” |
| | /replace = “t” |
| repeat region | 22029 . . . 22190 |
| | /rpt_family = “FRAM” |
| | /rpt_type = dispersed |
| variation | 22084 |
| | /gene = “IL4R” |
| | /frequency = “0.04” |
| | /replace = “a” |
| repeat region | 22202 . . . 22351 |
| | /rpt_family = “AluSg” |
| | /rpt_type = dispersed |
| variation | 22211 |
| | /gene = “IL4R” |
| | /frequency = “0.08” |
| | /replace = “g” |
| repeat region | 22491 . . . 22799 |
| | /rpt_family = “AluJo” |
| | /rpt_type = dispersed |
| variation | 22652 |
| | /gene = “IL4R” |
| | /frequency = “0.04” |
| | /replace = “” |
| variation | 22690 |
| | /gene = “IL4R” |
| | /frequency = “0.21” |
| | /replace = “a” |
| variation | 22738 |
| | /gene = “IL4R” |
| | /frequency = “0.01” |
| | /replace = “a” |
| variation | 22825 |
| | /gene = “IL4R” |
| | /frequency = “0.02” |
| | /replace = “a” |
| repeat region | 22877 . . . 22977 |
| | /rpt_family = “L2” |
| | /rpt_type = dispersed |
| variation | 22889 |
| | /gene = “IL4R” |
| | /frequency = “0.01” |
| | /replace = “c” |
| variation | 23026 |
| | /gene = “IL4R” |
| | /frequency = “0.08” |
| | /replace = “c” |
| variation | 23117 |
| | /gene = “IL4R” |
| | /frequency = “0.37” |
| | /replace = “a” |
| variation | 23171 |
| | /gene = “IL4R” |
| | /frequency = “0.02” |
| | /replace = “c” |
| variation | 23431 |
| | /gene = “IL4R” |
| | /frequency = “0.30” |
| | /replace = “c” |
| variation | 23474 |
| | /gene = “IL4R” |
| | /frequency = “0.30” |
| | /replace = “t” |
| variation | 23523 |
| | /gene = “IL4R” |
| | /frequency = “0.11” |
| | /replace = “c” |
| variation | 23525 |
| | /gene = “IL4R” |
| | /frequency = “0.02” |
| | /replace = “t” |
| variation | 23531 |
| | /gene = “IL4R” |
| | /frequency = “0.31” |
| | /replace = “c” |
| variation | 23539 |
| | /gene = “IL4R” |
| | /frequency = “0.04” |
| | /replace = “t” |
| variation | 23739 |
| | /gene = “IL4R” |
| | /frequency = “0.26” |
| | /replace = “c” |
| variation | 23959 |
| | /gene = “IL4R” |
| | /frequency = “0.43” |
| | /replace = “g” |
| variation | 23967 |
| | /gene = “IL4R” |
| | /frequency = “0.06” |
| | /replace = “a” |
| variation | 24255 |
| | /gene = “IL4R” |
| | /frequency = “0.02” |
| | /replace = “t” |
| variation | 24486 |
| | /gene = “IL4R” |
| | /frequency = “0.14” |
| | /replace = “g” |
| variation | 24629 |
| | /gene = “IL4R” |
| | /frequency = “0.06” |
| | /replace = “c” |
| variation | 24716 |
| | /gene = “IL4R” |
| | /frequency = “0.09” |
| | /replace = “c” |
| variation | 24718 |
| | /gene = “IL4R” |
| | /frequency = “0.08” |
| | /replace = “c” |
| variation | 24759 |
| | /gene = “IL4R” |
| | /frequency = “0.02” |
| | /replace = “t” |
| variation | 24798 |
| | /gene = “IL4R” |
| | /frequency = “0.04” |
| | /replace = “g” |
| variation | 24982 |
| | /gene = “IL4R” |
| | /frequency = “0.01” |
| | /replace = “a” |
| variation | 24992 |
| | /gene = “IL4R” |
| | /frequency = “0.01” |
| | /replace = “a” |
| variation | 25101 |
| | /gene = “IL4R” |
| | /frequency = “0.38” |
| | /replace = “a” |
| variation | 25346 |
| | /gene = “IL4R” |
| | /frequency = “0.39” |
| | /replace = “g” |
| variation | 25448 |
| | /gene = “IL4R” |
| | /frequency = “0.21” |
| | /replace = “t” |
| variation | 25776 |
| | /frequency = “0.39” |
| | /replace = “g” |
| repeat region | 25777 . . . 25890 |
| | /rpt_family = “LTR16C” |
| | /rpt_type = dispersed |
| repeat region | 25891 . . . 26433 |
| | /rpt_family = “MER41A” |
| | /rpt_type = dispersed |
| variation | 26042 |
| | /frequency = “0.01” |
| | /replace = “c” |
| variation | 26196 |
| | /frequency = “0.30” |
| | /replace = “g” |
| repeat region | 26434 . . . 26550 |
| | /rpt_family = “LTR16C” |
| | /rpt_type = dispersed |
| variation | 26457 |
| | /frequency = “0.01” |
| | /replace = “a” |
| variation | 26469 |
| | /frequency = “0.40” |
| | /replace = “t” |

Particularly relevant isoforms/variants/variations are indicated herein above in bold print.

The above depicted SEQ ID NO: 3 encodes for a polypeptide, comprising the herein described “I50V” (or “I75V”) SNP/variant. Said encoded polypeptide is shown in SEQ ID NO: 4, comprising the “V” at position 75 of the IL4R/CD124 polypeptide with a signal peptide of 25 amino acids (resulting in the “I75V” denomination).

(SEQ ID NO: 4)

MGWLCSGLLFPVSCLVLLQVASSGNMKVLQEPTCVSDYMSISTCEWKMNG
PTNCSTELRLLYQLVFLLSEAHTCVPENNGGAGCVCHLLMDDVVSADNYT
LDLWAGQQLLWKGSFKPSEHVKPRAPGNLTVHTNVSDTLLLTWSNPYPPD
NYLYNHLTYAVNIWSENDPADFRIYNVTYLEPSLRIAASTLKSGISYRAR
VRAWAQCYNTTWSEWSPSTKWHNSYREPFEQHLLLGVSVSCIVILAVCLL
CYVSITKIKKEWWDQIPNPARSRLVAIIIQDAQGSQWEKRSRGQEPAKCP
HWKNCLTKLLPCFLEHNMKRDEDPHKAAKEMPFQGSGKSAWCPVEISKTV
LWPESISVVRCVELFEAPVECEEEEEVEEEKGSFCASPESSRDDFQEGRE
GIVARLTESLFLDLLGEENGGFCQQDMGESCLLPPSGSTSAHMPWDEFPS
AGPKEAPPWGKEQPLHLEPSPPASPTQSPDNLTCTETPLVIAGNPAYRSF
SNSLSQSPCPRELGPDPLLARHLEEVEPEMPCVPQLSEPTTVPQPEPETW
EQILRRNVLQHGAAAAPVSAPTSGYQEFVHAVEQGGTQASAVVGLGPPGE
AGYKAFSSLLASSAVSPEKCGFGASSGEEGYKPFQDLIPGCPGDPAPVPV
PLFTFGLDREPPRSPQSSHLPSSSPEHLGLEPGEKVEDMPKPPLPQEQAT
DPLVDSLGSGIVYSALTCHLCGHLKQCHGQEDGGQTPVMASPCCGCCCGD
RSSPPTTPLRAPDPSPGGVPLEASLCPASLAPSGISEKSKSSSSFHPAPG
NAQSSSQTPKIVNFVSVGPTYMRVS

The present invention provides for a method of diagnosing and/or predicting joint destruction, in particular early joint destruction, rapidly erosive rheumatoid arthritis and/or accelerated joint destruction, said method comprising determining in a sample obtained from an individual the presence of an encoded IL-4 receptor (IL-4R) which comprises at the homologous position 75 of IL-4 receptor as depicted in SEQ ID NO: 2 a mutation, said mutation comprising the exchange from an isoleucine to a valine. As mentioned above, it is evident for the skilled artisan that also the determination of the wild-type (“I”) allele has predictive and/or diagnostic character, i.e. is of diagnostic relevance. Is, e.g. the wild-type allel (I50/I75) determined in a human patient, i.e. a patient suffering from rheumatoid arthritis (or a related disorder), is determined to have on position 50 or 75 of the IL4R as described herein the wild-type allele(s) I, it can be assumed that the disorder is of less erosive character or even non-erosive character in terms of joint destruction. In such a case, a less aggressive and/or moderate treatment/medical intervention is indicated. In contrast, in cases wherein the human individual/human patient has at least one mutant allele on position 50/75 of IL4R (i.e. at least one “V” allele) a more aggressive therapy is indicated since this mutant allele is the predictor for an erosive development and/or character of joint destruction. Accordingly, the marker provided herein is a predictor for an erosive course of (rheumatoid) arthritis versus a non-erosive course. The corresponding genetic disposition of an individual can, accordingly, influence the medical intervention, i.e. aggressive versus non- or less aggressive (e.g. DMARDs-treatment versus NSAIDs-treatment).

It will also be understood that further mutations might occur within the IL4R-gene, which as such may correlate with the presence of the mutation described herein. However, the appended examples also document that the herein defined “I50V”/“I75V” variant of IL4R (CD124) is solely indicative for the herein described early joint destruction, accelerated joint destruction and/or rapidly erosive rheumatoid arthritis. Particularly, it is envisaged that e.g. future SNPs or modification are detected within the IL4R-gene which are also correlated with the presence of the “I50V”/“I75V” variant as described herein.

The methods described herein, i.e. the methods of diagnosing and/or predicting early joint destruction, accelerated joint destruction, rapid/rapidly erosive rheumatoid arthritis etc. may comprise PCR, MALDI-TOF, ligase chain reaction, NASBA, restriction digestion, direct sequencing, nucleic acid amplification techniques, hybridization techniques or immunoassays. Also further techniques are envisaged. In accordance with this embodiment of the present invention, the diagnosis of a rapid erosive rheumatoid disorder can, e.g., be effected by isolating cells from an individual, and isolating the genomic DNA of said cells. Such cells can be collected from body fluids, skin, hair, biopsies and other sources. As documented in the appended examples, in particular peripheral blood is useful. Collection and analysis of cells from bodily fluids such as blood, urine and cerebrospinal fluid is well known to the art; see for example, Rodak, “Haematology: Clinical Principles & Applications” second ed., WB Saunders Co, 2002; Brunzel, “Fundamentals of Urine and Body Fluids Analysis”, WB Saunders Co, 1994; Herndon and Brumback (Ed.), “Cerebrospinal Fluid”, Kluwer Academic Pub., 1989. In addition, methods for DNA isolation are well described in the art; see, for example, Sambrook et al., “Molecular Cloning: A Laboratory Manual”, 3^{rd} edition, Cold Spring Harbor Laboratory, 2001.

Once DNA has been isolated, various oligonucleotide primers spanning the variant (I50V) coding region of IL4R (CD124) as described herein locus may be designed in order to amplify the genetic material by Polymerase Chain Reaction (PCR). Conventional methods for designing, synthesizing, producing said oligonucleotide primers and performing PCR amplification may be found in standard textbooks, see, for example Agrawal (Ed.), “Protocols for Oligonucleotides and Analogs: Synthesis and Properties (Methods in Molecular Biology, 20)”, Humana Press, 1993; Innis et al. (Ed.), “PCR Applications: Protocols for Functional Genomics”, Academic Press, 1999; Chen and Janes (Ed.), “PCR Cloning Protocols: From Molecular Cloning to Genetic”, 2^{nd} edition, Humana Press, 2002. Primers for the detection of IL4R variation(s) is/are also given in the appended examples. Once DNA has been amplified, nucleotide structure can be analysed by sequencing methods and compared to normal IL4R (CD124) DNA, i.e. comprising a codon encoding for “isoleucine” at position 75 of SEQ ID NO: 2. Sequencing may be performed manually by any molecular biologist of ordinary skills or by an automated sequencing apparatus. These procedures are common in the art, see, for example, Adams et al. (Ed.), “Automated DNA Sequencing and Analysis”, Academic Press, 1994; Alphey, “DNA Sequencing: From Experimental Methods to Bioinformatics”, Springer Verlag Publishing, 1997. Also RNA analysis may be employed in order to detect the presence or absence of the herein described I50V/I75V variant of IL4R (CD124). Also RT-PCR methods and techniques may be employed in accordance with this invention.

Detection and analysis of variations, in particular the variant described herein, in IL4R/CD124 encoding nucleic acid sequences can also be performed using amplification refractory mutation system (ARMSTM), amplification refractory mutation system linear extension (ALEXTM), single-strand conformation polymorphism (SSCP), heteroduplex analysis, PCR-SSCP, fluorescent SSCP in an automated DNA sequencer, denaturing gradient gel electrophoresis, RNase protection assays, detection of mutations by sequence specific oligonucleotide hybridization, chemical cleavage methods, enzyme mismatch cleavage methods, cleavage fragment length methods, allele-specific oligonucleotide hybridization on DNA chips or wafers or arrays, and other such methods known in the art, see, for example Nollau et al, Clin. Chem. 43 (1997), 1114-1128; Burczak and Mardis (Ed.), “Polymorphism Detection & Analysis Techniques”, Eaton Pub Co, 2000; Cotton et al. (Ed.), “Mutation Detection: A Practical Approach”, Irl Press, 1998; Taylor (Ed.), “Laboratory Methods for the Detection of Mutations and Polymorphisms in DNA”, CRC Press, 1997.

Therefore, the person skilled in the art is readily in a position to work the present invention over the full scope. SNPs and single point mutations, in particular the mutation (I50V/I75V) described herein, can, inter alia, also be detected using various methods, like the analysis of restriction fragment length polymorphism and mobility shift of single-stranded DNAs (Botstein, 1980 Am J Hum Genet 32: 314-31; Orita, 1989 PNAS 86: 2766-2770). Furthermore, PCR related methods or methods used in genotyping studies including automated DNA sequencing may provide important information about the occurrence of SNP's and single point mutations (Saiki, 1989 PNAS 86: 6230-6234; Verpy, 1994 PNAS 91: 1873-1877). Corresponding techniques are also shown in the appended examples.

Alternatively, fluorescently labelled allele- or SNP-specific hybridization probes are methods which allow rapid and reliable detection of DNA point mutations. Typical hybridization probes may comprise a short recognition sequence and two fluorophores whose spatial separation is governed by strand-specific hybridization. One unit serves as the reporting element and is modulated (turned on/off) by the proximity of the second by using Fluorescence resonance energy transfer (FRET) technology. Non-limiting examples include stem-loop molecular beacons and TaqMan-type probes, which are of current interest in real-time SNP screening (Bonnet, 1999 PNAS 96: 6171-6176; Marras, 1999 Genet Anal 14: 151-156; Livak, 1999 Genet Anal 14: 143-149; Gaylord, 2005 PNAS 102: 34-39). High-throughput screening at the nucleotide level for previously identified point mutations may be accomplished by using genotyping chips (Wang, 1998 Science 280: 1077-1082).

On the protein level, point mutations may be detected by using mass spectrometry techniques such as electrospray ionization-time of flight (ESI-TOF) or matrix-assisted laser desorption/ionization-time of flight (MALDI-TOF) analysis (Taranenko, 1996 Genet Anal 13: 87-94; Ball, 1998 Protein Sci 7: 758-764). It is also envisaged that antibody-based techniques be employed, like Western-blot analysis, ELISA, RIA, IRMA and the like. These techniques may employ specific antibodies directed against the mutant “V” or the wild-type “I” variant of IL-4R. The generation of antibodies is well-known in the art, see, inter alia, Harlow & Lane, “Antibodies: A Laboratory Manual” (1988) Cold Spring Harbor Laboratory Press. In accordance with the present invention, the term “nucleic acid sequence” means the sequence of bases comprising purine- and pyrimidine bases which are comprised by nucleic acid molecules, whereby said bases represent the primary structure of a nucleic acid molecule. Nucleic acid sequences include DNA, cDNA (e.g. obtained from spliced and/or unspliced mRNA), genomic DNA, RNA, synthetic forms and mixed polymers, both sense and antisense strands, or may contain non-natural or derivatized nucleotide bases, as will be readily appreciated by those skilled in the art.

In the context of the present invention the term “molecular variant”/“molecular isoform” IL4R/CD124 gene or protein as used herein means that said IL4R (CD124) gene or protein differs from the wild type IL4R (CD124) gene or protein by at least one nucleotide or amino acid substitution, deletion and/or addition. Variations and isoforms of IL4R are also provided above in context of SEQ ID NO. 3. The cDNA sequences of the wildtype IL4R gene as used herein is shown in SEQ ID NO: 1, and the wild-type IL4R amino acid sequence is shown in SEQ ID NO: 2. It will be understood that the methods of the present invention include that instead of determining the presence of a nucleotide sequence having the mutation described herein it is, alternatively, also possible to detect the presence of the encoded variant polypeptide of the described IL4R variant (namely the exchange from an isoleucine to a valine at position 75 of the IL4R as shown in SEQ ID NO: 2 or a similar or homologous substitution in a homologous variant (allelic variant) of the ILR as shown in SEQ ID NO: 2). As pointed out above, also the determination of the wild-type allel (I50/I75 and their corresponding encoding nucleotides in the coding nucleotide sequence) is of high diagnostic relevance since it allows for the determination of a less or even non-erosive character of the disease, in particular in RA.

A nucleotide sequence of human IL4R/CD124 is disclosed in NCBI nucleotide entry NCBI NM_{—}000418 (gi: 56788409). Under the accession number AF421855 the complete cds of the IL4R gene is provided and specific variations, inter alia, the herein defined I50V/I75V variation is described. However, in the context of the present invention, the sequence as depicted in SEQ ID No: 1 is referred to as the “wildtype” sequence.

When used herein, the term “polypeptide” means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The terms polypeptide and protein are often used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature.

When used in the context of the present invention the term “a predisposition for a disease” in particular the early joint destruction and/or rapidly erosive rheumatoid arthritis includes a tendency or susceptibility to develop a certain disease that can be triggered under certain conditions, e.g. influence of the environment, personal circumstances of a subject, such as the nutritional behavior of a subject. The tendency or susceptibility to develop a certain disease is associated with a genotype that increases the risk for developing a disease, if other certain conditions, such as those mentioned above, are present. Genetic testing is able to identify subjects who are genetically predisposed to certain health problems as defined herein. The disease for which a subject has a tendency or susceptibility to develop is preferably a disease related to the presence of a molecular variant of IL4R/CD124 as described herein and comprises early joint destruction, accelerated joint destruction and/or rapidly erosive rheumatoid arthritis.

The term “position” when used in accordance with the present invention means the position of either an amino acid within an amino acid sequence depicted herein or the position of a nucleotide within a nucleic acid sequence depicted herein. The term “corresponding” as used herein also includes that a position is not only determined by the number of the preceding nucleotides/amino acids. The exact position of a given nucleotide in accordance with the present invention which may be substituted may vary due to deletions or additional nucleotides elsewhere in the IL4R/CD124 promoter or gene. Thus, under a “corresponding position” in accordance with the present invention it is to be understood that nucleotides may differ in the indicated number (for example position “75” of the full IL4R (with signal sequence) or position 50 of the mature protein (without signal sequence) or position 465 of the nucleotide sequence representing the cDNA nucleotide sequence of a wildtype IL4R as shown in SEQ ID NO: 1 but may still have similar neighboring nucleotides. Said nucleotides which may be exchanged, deleted or comprise additional nucleotides are also comprised by the term “corresponding position”.

The position with respect to nucleotide sequences mentioned herein refers to the sequence shown in SEQ ID NO: 1. This sequence represents the cDNA nucleic acid sequence of the IL4R gene encoding the interleukin 4 receptor (CD124). It is possible for the skilled person to identify the position in the cDNA sequence corresponding to a position in SEQ ID NO: 1 by aligning the sequences.

In order to determine whether an amino acid residue or nucleotide residue in a given IL4R sequence corresponds to a certain position in the amino acid sequence or nucleotide sequence of SEQ ID NO: 1 or 2, the skilled person can use means and methods well-known in the art, e.g., alignments, either manually or by using computer programs such as those mentioned further down below in connection with the definition of the term “hybridization” and degrees of homology.

For example, BLAST2.0, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be used to search for local sequence alignments. BLAST produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

% sequence identity×% maximum BLAST score/100

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson, Nucl. Acids Res. 2 (1994), 4673-4680) or FASTDB (Brutlag Comp. App. Biosci. 6 (1990), 237-245), as known in the art.

As described herein, the present invention describes a specific informative variant of one IL4R gene which means that at a certain position (465 of the full cDNA sequence as shown in SEQ ID NO: 1) of the wild-type IL4R/CD124 nucleotide sequence a nucleotide is replaced by a different nucleotide as described herein. The term “IL4R gene” or “CD124 gene” includes the 5′UTR, exons, introns and the 3′UTR of IL4R/CD124 whose cDNA wild-type nucleotide sequences is shown in SEQ ID NO: 1. The term “is replaced by a different nucleotide” when used in the context of the present invention means that the nucleotide residing at a position of the wild-type IL4R nucleic acid sequences mentioned herein is replaced by a nucleotide which is not the same nucleotide as that residing on the said position of the wild-type IL4R nucleic acid sequences.

The present invention also relates to the use of nucleic acid molecules which hybridize to one of the above described nucleic acid molecules and which shows a mutation as described hereinabove in the methods and means provided herein.

The term “hybridizes” as used in accordance with the present invention may relate to hybridizations under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which code for a IL-4 receptor molecule (CD124) fragment thereof, and which have a length of at least 12 nucleotides, preferably at least 15, more preferably at least 18, more preferably of at least 21 nucleotides, more preferably at least 30 nucleotides, even more preferably at least 40 nucleotides and most preferably at least 60 nucleotides and which are capable to hybridize to a region to be amplified by recombinant techniques, said region comprising a mutation in position 465 as defined herein in context of SEQ ID NO: 1 (the cDNA wild-type IL4R sequence) and said position to be amplified or said region whereto these fragments hybridize to comprise the herein defined diagnostic mutation. Also indicative is the case where the herein defined mutation is not detected, i.e. wherein no early joint destruction/rapid (rapidly) erosive rheumatoid arthritis and/or accelerated joint destruction is to be detected, diagnosed and/or predicted.

Hybridizing sequences in accordance with this invention may be specific primers and/or probes useful in the detection of the herein described variant/mutation of the IL4R (CD124) “I50V”/“I75V”. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementartity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity with a nucleic acid sequence as described above encoding an IL4R (CD124) protein having a described mutation. Moreover, the term “hybridizing sequences” preferably refers to sequences encoding an IL4R (CD124) protein having a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity with an amino acid sequence of an IL4R (CD124) variant comprising the herein described mutation/variation isoleucine to valine as described herein above.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands.

Moreover, the present invention also relates to the use of nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. Also these nucleic acid molecules are useful in the methods and means (like diagnostic kits) provided herein.

In another embodiment of the methods described herein, the “I50V”/“I75V” SNP or point mutation is detected in homozygous alleles/homozygosity, in particular said mutation/SNP is detected in a sample from a homozygous individual. As discussed herein, the detection in a homozygous individual is advantageous, however, also the detection of the I50V/I75V SNP/variant in a heterozygous subject or individual is conclusive and may successfully be employed in the methods and means of the invention. However, as pointed out above, also the determination of the wild-type allel(s) (I50/I75) is/are of high diagnostic value, since it allows for the prediction whether the diseases is of less (or even non-) erosive character. Individuals which are diagnosed in accordance with this invention to be bearer of at least one mutant allel as described herein (i.e. V501V75 of IL4R) will suffer from a more erosive disease and more aggressive medical intervention is indicated in these patients. A more aggressive medical intervention in this erosive joint destruction is e.g. anti-TNF treatment, anti-IL-15 treatment, anti-IL-6 treatment, treatment with CTLA-4Ig, depletion of B-cells (like with antibodies directed against CD20 or CD22; see e.g. Rituximab®), or the “DMARD-therapy”.

Some patients with rheumatoid arthritis (RA) are more likely than others to show joint damage within the first 2 years of disease onset (early onset and early joint destruction). Some studies have shown that patients who have active RA in multiple joints and those who show high levels of certain antibodies based on blood tests have a 70% chance of developing joint damage within this early time period. The present invention provides for a reliable test in particular for these subjects/patients, since it is now possible to scrutinize now at an early point of time whether a given patient will suffer from erosive, early joint destruction or whether the diseases is less or even non-erosive, in case he/she is “wild-type” for the I50/I75 allel of IL4R as described herein.

In the context of the present invention, the term “subject” means an individual in need of a treatment of an affective disorder. Preferably, the subject is a mammalian, particularly preferred a human, a horse, a camel, a dog, a cat, a pig, a cow, a goat or a fowl. The present invention is particularly useful in the determination whether a given or suspected RA of a human patient is erosive or non-erosive or whether a joint destruction is to be expected early on. If this is the case, since the human patient is either homo- or heterozygous for the herein described point mutation (V50N75 of IL4R), an aggressive medical intervention is indicated.

As discussed herein and shown in the experimental part, the present invention provides for the prediction and/or diagnosis of erosive joint destruction, in particular an early joint destruction. Said joint destruction is in particular associated with early erosion(s) and/or a higher severity in the progression of rheumatoid arthritis (RA). The means and methods provided herein are particularly useful, since a positive result, i.e. the presence of the I50V/I75V IL4R SNP/mutation will ideally prompt an early and more aggressive therapeutic intervention in the positively screened/scored individuals. As pointed out herein, is a given subject patient diagnosed as comprising the herein defined “wild-type” allel (I50/I75 of IL4R), a more moderate or less aggressive therapy is indicated. Early prognosis of an early or an erosive joint destruction may prevent further damage, like bone destruction. “Aggressive treatment” that has been shown to prevent joint damage includes treatment with substances to inhibit the activity of TNF, treatment with substances to inhibit the activity of IL-1, treatment with substances to inhibit T cell costimulation (such as neutralizing antibodies to CD28 or fusion proteins such as CTLA-4Ig or derivatives thereof), treatment with substances to inhibit the activity of IL-6, treatment with substances to inhibit the activity of IL-15, treatment with substances to inhibit the activity of IL-17 or treatment with substances to deplete B cells (such as antibodies to CD20 or CD22, like Rituximab®&, Rituxan® and MabThera®, Epratuzumab® and the like). Aggressive treatment also refers to any combination of two or more so called “disease modifying anti-rheumatic drugs (DMARDs)”, such as methotrexate, cyclosporine, azathioprine, sulfasalazine, mycophenolate mofetil, chloroquine, hydroxychloroquine, gold, D-penicillamine, bucillamine, cyclophosphamide and leflunomide. In particular, when the herein described mutant “I50V”/“I75V” SNP (mutant allel V50N75 of IL4R) is detected in a homozygous setting, the treatment of choice would preferably be an “aggressive treatment” as described above. Particularly, patients who may show signs of bone and/or joint destruction but who appear to be homozygously “wild-type” at position 50 or 75 of the IL4R α-chain as described herein (i.e. “I50V”/“I75V”) may be treated with commonly used non-steroidal anti-inflammatory drugs/NSAIDs, in particular during an early phase of the disease. However, it is of note that also patients with a “wild-type” genetic setting of IL4R on position I50/I75 of 114R as described herein, but already suffering from bone erosions should be treated aggressively.

Yet, such homozygous “wild-type” (I50/I75) subject or patients who are diagnosed early on (for example during their first visit at the attending physician or rheumatologist) as suffering from RA can be treated less aggressively, for example with non-steroidal anti-inflammatory drugs/NSAIDs such as acetylsalicylic acid, indomethazine, acemathazine, proglumethazine, mefenaminic acid, diclofenac, aceclofenac, lonacolac, ibuprofen, ketoprofen, naproxene, flurbiprofen, tiaprofenic acid, piroxicam, tenoxicam, meloxicam, lomoxicam, celecoxib, etoricoxib, lumiracoxib, phenylbutazone, mofebutazone or solely with (low-dose) corticosteroids. Accordingly, with the teaching of the present invention, different potential treatments may be offered to different patients, in particular potential arthritis patients, depending on the allelic setting in position 50 (75) of the amino acid sequence of IL4R.

Wild-type individuals (“I50I”/“I75I” in the IL4R SNP, e.g. “I” on the herein defined position 50 or 75 of alpha-chain IL4R protein sequence;) showing no signs of joint and/or bone destruction may be treated with, inter alia, NSAIDs and/or low dose steroids, heterozygous patients (comprising one “I50V”/“I75V” allele/SNP in IL4R) may be treated with one DMARD, low dose steroids or NSAIDs (and should be frequently controlled by radiography) and homozygous patients (comprising the “I50V”/“I75V” SNP in both IL4R alleles) should be treated with “aggressive therapy” with biologicals as identified above, e.g. TNF neutralizing agents and/or a combination of two or more DMARDs in order to prevent bone erosions.

Without being bound by theory, the following treatment regime is envisaged as a non-limiting example. The determination of the allels at the IL4R I50V locus can be used for a stratification of the therapy in patients with recent onset rheumatoid arthritis. If a patient with recent onset rheumatoid arthritis has not developed bone erosions at the time of presentation, a patient with homozygosity for the wildtype allel (I50I/I75I) could be treated conservatively with low dose corticosteroids (for example and non-limiting:<10 mg/d) and NSAIDS, but without DMARDs or biologics. The expected mild course of the disease would also merit control visits in intervals of six months durings the first two years of the disease. A patient with a heterozygosity at the IL4R I50V/I75V locus (i.e. one “mutant allel V50/V75) would require more aggressive treatment regimens, such as medium doses of corticosteroids at the beginning (for example and non-limiting: 20 mg/d initially and a reduction to 10 mg over a period of six weeks), NSAIDS and one DMARD, such as methotrexate. The follow-up visits would be scheduled in three to six months intervals. In case of homozygosity for the mutant allele (V50V/V75V) the patients should be started with most aggressive therapy, such as high dose steroids (for example and non-limiting: 50 mg/d with a reduction to 10 mg/d over a period of 6 weeks), a DMARD such as methotrexate or biologics (such as TNF inhibitors). Follow-up visit should be scheduled every six weeks to three months, and any indication of persistently active disease should result in the decision to add a second DMARD or to switch to a biologic, like Rituximab or Abatacept.

As pointed out above, the method of detecting the herein described I50V/I75V SNP of IL4R may comprise detection on the protein as well as the nucleic acid molecule level and common methods comprise PCR-technology, ligase-chain reaction, NASBA, restriction digestion, direct sequencing, nucleic acid amplification techniques, MALDI, MALDI-TOF, hybridization techniques and/or immuno assays. Accordingly, in e.g. immunoassays specific antibodies and/or binding molecules directed against an epitope, comprising the defined “V” in context of the surrounding amino acids may be employed. Therefore, preferably binding molecules and/or antibodies detecting an epitope comprise, e.g. the amino acid stretch SEAHTCVPENN or a part thereof may be employed. The specific antibody or binding molecule may also comprise a binding molecule that specifically detects a conformational and/or split epitope which is present in the I50V variant polypeptide described herein and which is not present in a wild-type IL4R α-chain or a homologous IL4R a-chain which does not comprise the “I50V”/“I75V” SNP/variant. Such a binding molecule may also be a specific aptamer and the like.

Antibodies to be employed in the present methods of the invention are not limited to polyclonal or monoclonal antibodies or antibodies derived from immunization of e.g. corresponding (laboratory) animals. The term “antibody” also relates to modified antibodies (like humanized antibodies, CDR-grafted antibodies, single chain antibodies) or antibody fragments, like F(ab) fragments and the like.

Therefore, in context of the present invention, the term “antibody molecule” or “antibody” relates to full immunoglobulin molecules, preferably IgMs, IgDs, IgEs, IgAs or IgGs, more preferably IgG1, IgG2, IgG2a, IgG2b, IgG3 or IgG4 as well as to parts of such immunoglobulin molecules, like Fab-fragments or V_{L}-, V_{H}- or CDR-regions. Furthermore, the term relates to modified and/or altered antibody molecules, like chimeric, CDR-grafted and humanized antibodies. The term also relates to modified or altered monoclonal or polyclonal antibodies as well as to recombinantly or synthetically generated/synthesized antibodies. The term also relates to intact antibodies as well as to antibody fragments/parts thereof, like, separated light and heavy chains, Fab, Fab/c, Fv, Fab′, F(ab′)₂. The term “antibody molecule” also comprises antibody derivatives, the bifunctional antibodies and antibody constructs, like single chain Fvs (scFv) or antibody-fusion proteins.

The invention also relates to the use of specific probes and/or primers, specific antibody molecules or specific binding molecules (like aptamers) in the preparation of a diagnostic composition for diagnosing and/or predicting joint destruction, early joint destruction and/or accelerated joint destruction and/or the erosive character of the disease Said joint destruction may be associated with early erosion(s) and/or a higher severity in the progression of rheumatoid arthritis (RA).

In a further aspect, the present invention relates to the use of specific probes, like nucleic acid probes, primers as well as specific binding molecules, like antibodies or aptamers in the preparation of a diagnostic composition. Accordingly, said diagnostic composition may, inter alia, comprise one or more of the nucleic acid sequences as defined herein above as well as other specific reagents, like specific antibodies.

The corresponding probes/primers/antibodies and/or binding molecules comprised in the diagnostic composition of the present invention can also be packaged in a “kit”. Accordingly, the present invention also relates to a kit comprising specific probes and/or primers for diagnosing and/or predicting joint destruction, early joint destruction and/or accelerated joint destruction, said probes and/or primers being capable of detecting at least one nucleic acid sequence selected encoding for point mutation as described herein, i.e. the exchange from an isoleucine to a valine on position 50 (or 75) of the herein shown IL4R (and its corresponding isoforms and variants). Also part of this invention is a kit comprising antibodies, binding molecules, aptamers and the like which are capable of selectively binding to an encoded protein (or fragment thereof) which comprises the point mutation as described herein, e.g the I50V/I75V mutation of IL4R.

The diagnostic composition or kit of the present invention optionally comprises suitable means for detection. The nucleic acid molecule(s), or polypeptide(s) described above are, for example, suitable for use in immunoassays in which they can be utilized in liquid phase or bound to a solid phase carrier. Examples of well-known carriers include glass, polystyrene, polyvinyl ion, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

Solid phase carriers are known to those in the art and may comprise polystyrene beads, latex beads, magnetic beads, colloid metal particles, glass and/or silicon chips and surfaces, nitrocellulose strips, membranes, sheets, duracytes and the walls of wells of a reaction tray, plastic tubes or other test tubes. Suitable methods of immobilizing nucleic acid molecule(s), vector(s), host(s), antibody(ies), aptamer(s), polypeptide(s), etc. on solid phases include but are not limited to ionic, hydrophobic, covalent interactions or (chemical) crosslinking and the like. Examples of immunoassays which can utilize said compounds of the invention are competitive and non-competitive immunoassays in either a direct or indirect format. Commonly used detection assays can comprise radioisotopic or non-radioisotopic methods. Examples of such immunoassays are the radioimmunoassay (RIA), the sandwich (immunometric assay) and the Northern or Southern blot assay. Furthermore, these detection methods comprise, inter alia, IRMA (Immune Radioimmunometric Assay), EIA (Enzyme Immuno Assay), ELISA (Enzyme Linked Immuno Assay), FIA (Fluorescent Immuno Assay), and CLIA (Chemiluminescent Immune Assay). Furthermore, the diagnostic compounds of the present invention may be employed in techniques like FRET (Fluorescence Resonance Energy Transfer) assays.

Appropriate labels and methods for labeling are known to those of ordinary skill in the art. Examples of the types of labels which can be used in the present invention include inter alia, fluorochromes (like fluorescein, rhodamine, Texas Red, etc.), enzymes (like horse radish peroxidase, β-galactosidase, alkaline phosphatase), radioactive isotopes (like 32P, 33P, 35S or 125I), biotin, digoxygenin, colloidal metals, chemi- or bioluminescent compounds (like dioxetanes, luminol or acridiniums).

A variety of techniques are available for labeling biomolecules, are well known to the person skilled in the art and are considered to be within the scope of the present invention and comprise, inter alia, covalent coupling of enzymes or biotinyl groups, phosphorylations, biotinylations, random priming, nick-translations, tailing (using terminal transferases). Such techniques are, e.g., described in Tijssen, “Practice and theory of enzyme immunoassays”, Burden and von Knippenburg (Eds), Volume 15 (1985); “Basic methods in molecular biology”, Davis L G, Dibmer M D, Battey Elsevier (1990); Mayer, (Eds) “Immunochemical methods in cell and molecular biology” Academic Press, London (1987); or in the series “Methods in Enzymology”, Academic Press, Inc.

Detection methods comprise, but are not limited to, autoradiography, fluorescence microscopy, direct and indirect enzymatic reactions, etc.

Said diagnostic composition may be used for methods for detecting the presence and/or abundance of a nucleic acid molecule described herein, i.e. a nucleic acid molecule comprising a coding sequence which leads to the I50V (I75V) variant of IL4R (CD124) as described herein, in a biological and/or medical sample and/or for detecting expression of such a nucleic acid molecule (e.g. by determining the mRNA or the expressed polypeptide). As mentioned above, the present invention also relates to a kit comprising the diagnostic composition as described herein or the nucleic acid molecule, the polypeptide the primer or pair of primers of the invention (the mentioned primers are also described in the appended examples and herein below) or the molecule as identified or characterized in a method herein below of the present invention.

Advantageously, the kit of the present invention further comprises, optionally (a) reaction buffer(s), storage solutions and/or remaining reagents or materials required for the conduct of scientific or diagnostic assays or the like. Furthermore, parts of the kit of the invention can be packaged individually in vials or bottles or in combination in containers or multicontainer units.

The kit of the present invention may be advantageously used as diagnostic kits, as research tools or therapeutic tools. Additionally, the kit of the invention may contain means for detection suitable for scientific, medical and/or diagnostic purposes. The manufacture of the kits follows preferably standard procedures which are known to the person skilled in the art.

A specific probe to be used may be 5′-ACACGTGTATCCCTG-3′ (SEQ ID NO: 5) for the detection of “150” allele or 5′-CACGTGTGTCCCTG-3′ (SEQ ID NO: 6) for the detection of “V50” allele.

As documented in the examples primer or primer pairs to be employed in the inventive method or in the corresponding uses may be determined by methods known in the art. Such primers/primer pairs may comprise 5′-ACCCAGCCCCTGTGTCT-3′ (SEQ ID NO: 7) (forward primer), 5′-CGCGCCTCCGTTGTTC-3′ (SEQ ID NO: 8) (reverse primer).

Also comprised in the present invention is the use of the herein described probes/primers/antibodies/binding molecules/aptamers in automated diagnostic devices, like diagnostic robotors, automated analysers, automated PCR assays, automated detection technologies (like nucleic acid hybridization to sequencing, automated ELISA tests. Automated assays known to the skilled artisan are discussed and the person skilled in the art can easily adopt the corresponding assay system to the measurement of the herein disclosed marker/SNP/allelic situation relating to the herein described IL4R single nucleotide polymorphism I50V/I75V. Examples of such automated systems comprise but are not limited to Beckman-Coulter Access, Abbott ARCHITECT and AxSYM, Bayer Advia Centaur, DPC IMMULITE 2000, and Roche Modular Analytics E170 Assay systems and methods in clinical chemistry to be employed in context of this invention are also described in Fraser, C G (1986) “Interpretation of Clinical Chemistry Laboratory Data” Blackwell; Mayne, P D (1994) “Clinical Chemistry in Diagnosis and Treatment” 6th edition. Arnold; Marshall, W J (1996) Clinical Chemistry (3rd) Gower d edition; Price, C P (1990) “Principles and Practice of Immunoassay” Stockton Press; Roitt, I M (1993) “Essential Immunology” Blackwell; Teitz, N W (1996) “Clinical Guide to Laboratory Tests. Saunders”; Walmsley, R N, Watkinson, L R, Koag, E S C (1989). “Cases in Chemical Pathology—a Diagnostic Approach” P. G. Publishing; Whitby, L G, Smith, A F, Beckett, G J (1988) “Cases in Clinical Biochemistry” Blackwell; Burtis and Ashwood, (1999) “Tietz Textbook of Clinical Chemistry” SAUNDERS; or Bishop, (2005) “Clinical Chemistry: Principles, Procedures, Correlations”, Lippincott Williams & Wilkins. PCR technology or hybridization techniques in scientific approaches as well as in diagnosis are, inter alia, described in McPherson and Moller (2006), PCR (The Basics)”, Innis et al (1989), “PCR Protocols: A Guide to Methods and Applications”, Academic Press.

Therefore, the person skilled in the art is readily in a position to work and carry out the present invention, since the determination of a single nucleotide polymorphism is within the normal and routine skill of the skilled artisan. The analysis, as pointed out above, can be carried out on biological samples obtained form the subject, in particular DNA or RNA samples; see also appended examples wherein, for example, DNA isolation and corresponding genotyping is described in an illustrative manner). However, also the determination of the exchanged amino acid “V” versus “I” in IL4R by methods related to protein diagnosis is invisaged and part of this invention.

The figures show:

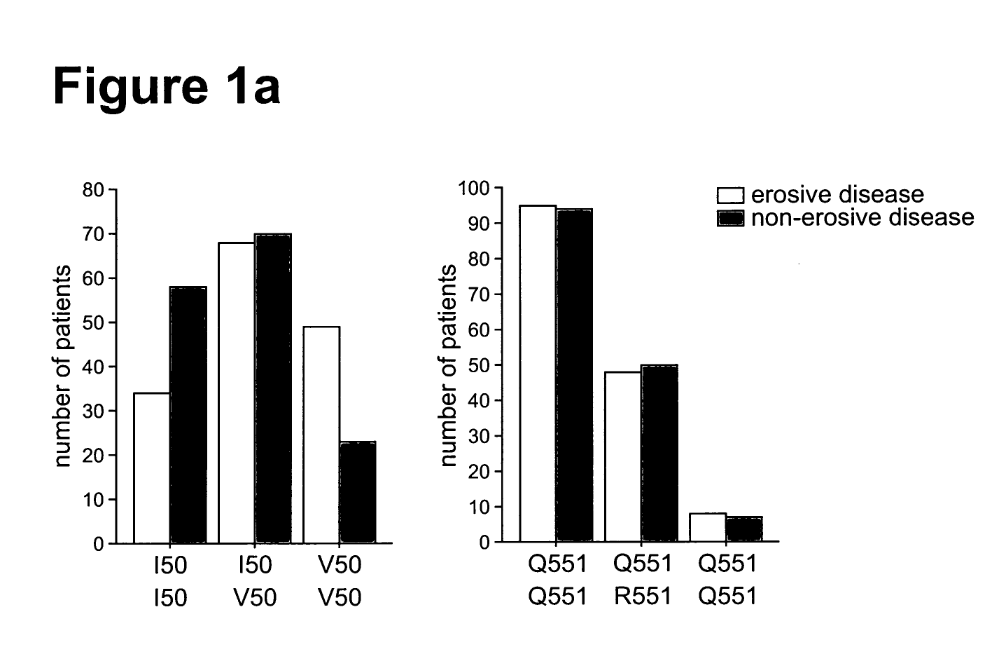

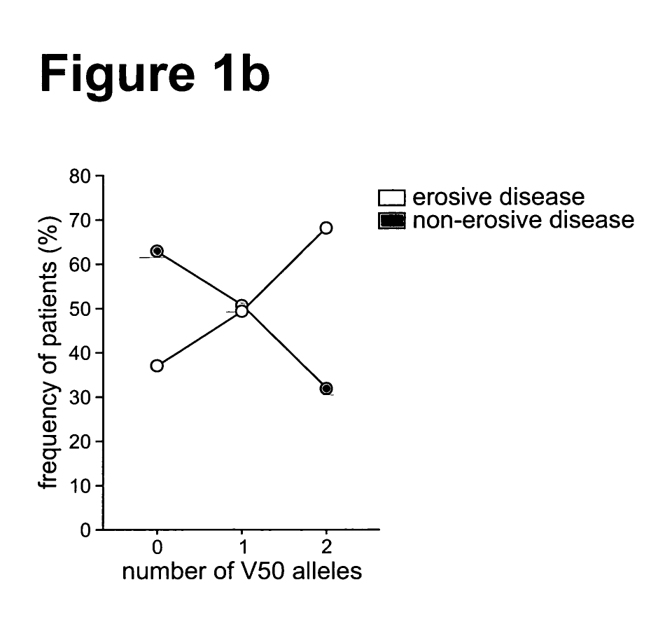

FIG. 1. Contribution of IL4R to the development of erosive RA. (A) Numbers of RA patients with erosive (white bars) or non-erosive (black bars) disease in different I50V or Q551R SNP IL4R groups are shown. (B) Frequencies of RA patients with erosive (white circles) or non-erosive (black circles) disease within different I50V IL4R SNP groups were calculated. Zero V50 alleles correspond to the I50I group, one V50 allele corresponds to the I50V group, and two V50 alleles correspond to the V50V group of the patients.

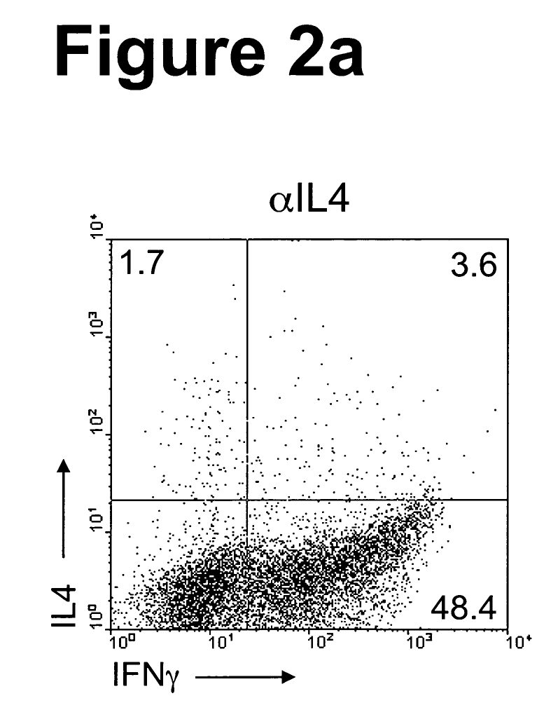

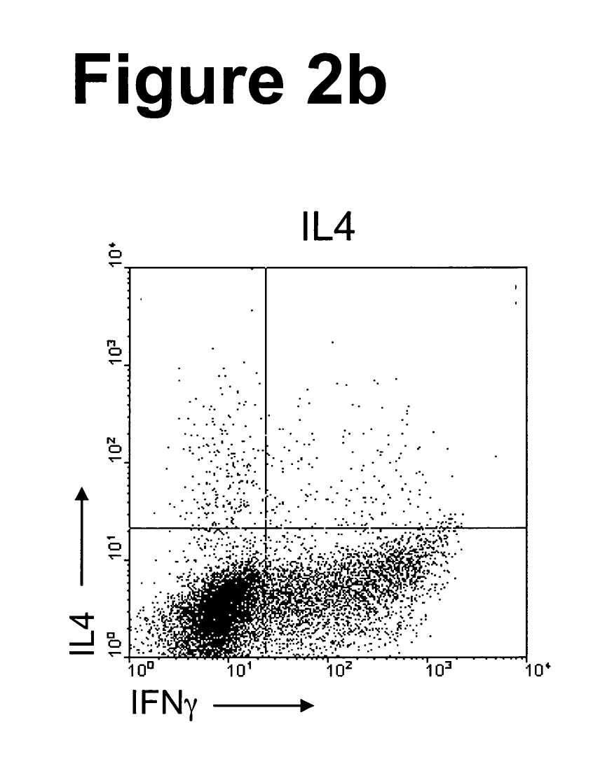

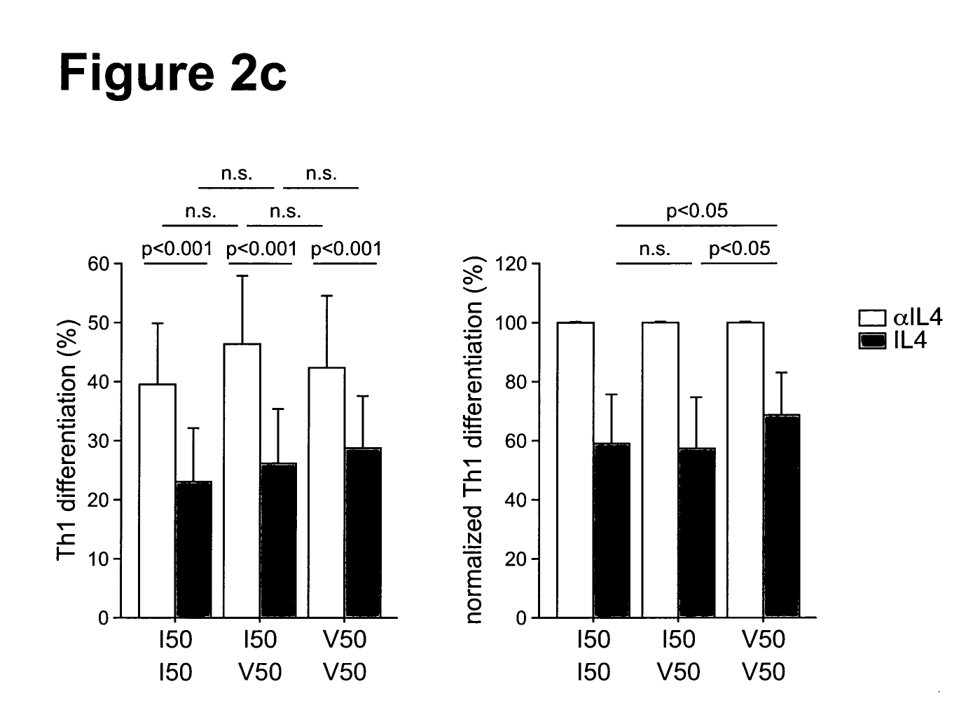

FIG. 2. Diminished IL4 effect on Th1 cell differentiation from V50 homozygous individuals. (A, B) Representative intracellular staining patterns of CD4 T cells that had been cultured in the presence of anti-IL4 (“αIL4”) (A) or IL4 (B) are demonstrated. (C) The left panel shows frequencies of IFNγ-producing Th1 cells after priming with anti-IL4 (black bars) or IL4 (white bars) in three groups of healthy individuals with the known I50V SNP genotype. For each individual group, Th1 cell frequencies were later normalized for the Th1 cell frequency in the respective anti-IL4-containing culture (right panel). Results are indicated as mean±SD of 25, 28, and 23 independent experiments, respectively, using cells from different healthy donors.

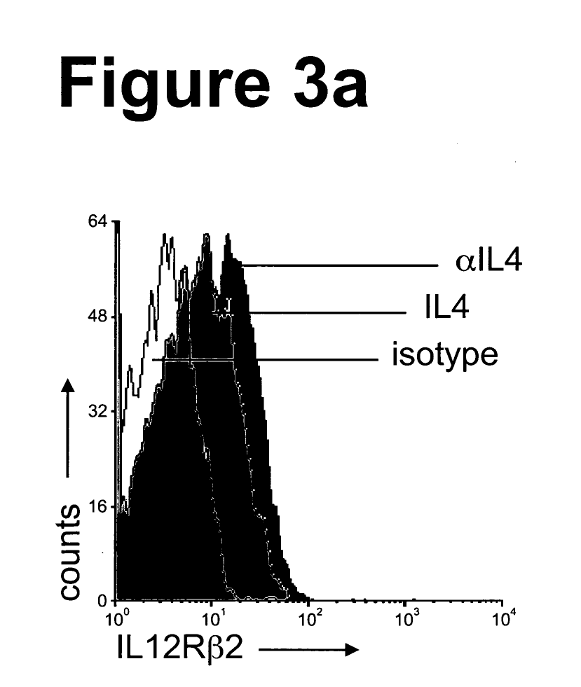

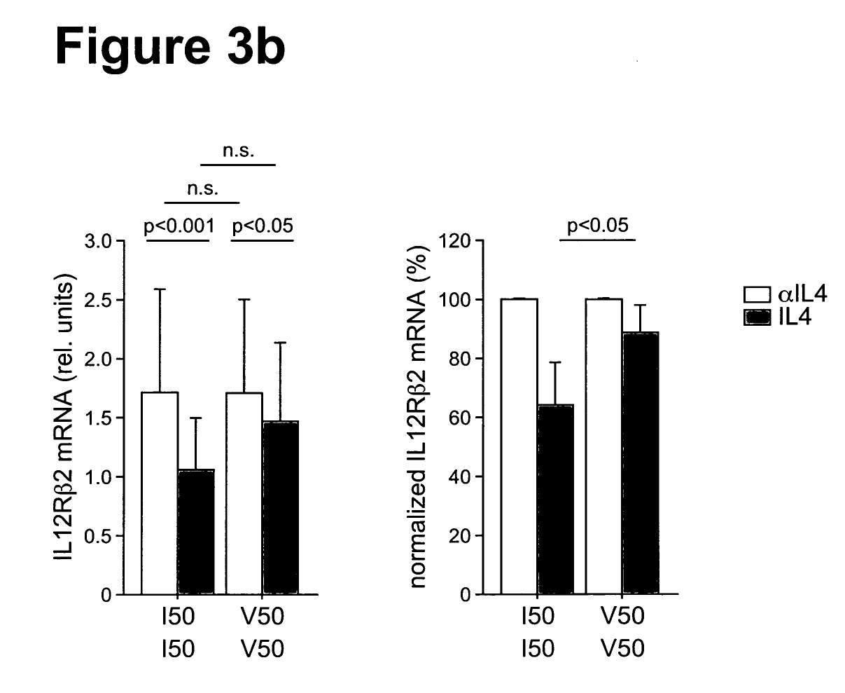

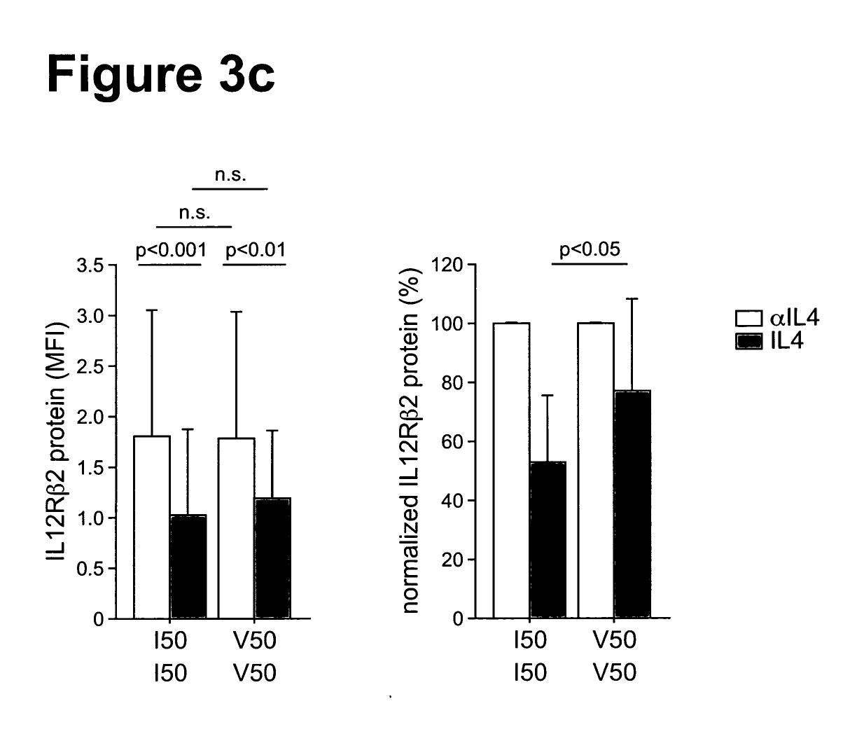

FIG. 3. Diminished IL4 effect on the expression of IL12Rβ2 from V50 homozygous individuals. (A) Representative IL12Rβ2 staining patterns of CD4 T cells that had been cultured in the presence of anti-IL4 (“αIL4”) or IL4 are demonstrated. The open histogram represents the isotype control. (B) The left panel shows relative expression of IL12Rβ2 mRNA in cells cultured in the presence of anti-IL4 (white bars) or IL4 (black bars). On the right panel the values were normalized for the relative expression of IL12Rβ2 mRNA in cells cultured in the presence of anti-IL4. Results are indicated as mean±SD of 6 independent experiments each using cells from different healthy donors. (C) The left panel shows surface expression of IL12Rβ2 on cells cultured in the presence of anti-IL4 (white bars) or IL4 (black bars). On the right panel the values were normalized for IL12Rβ2 expression on cells cultured in the presence of anti-IL4. Results are indicated as mean±SD of 16 independent experiments each using cells from different healthy donors. MFI, mean fluorescence intensity.

The Examples illustrate the invention.

### EXAMPLES

In the present study, the role of two IL4R SNPs (I50V and Q551R) in RA susceptibility and severity in an association study with 371 controls and 471 well-characterized RA patients with erosive disease was evaluated. Radiographs of the hands and feet two years after the disease onset were used to assess disease progression in RA since erosions of the hands and feet are most characteristic for RA and joint destruction is directly associated with work disability and poorer functional status (Kirwan (2001) J Rheumatol, 28(4):881-886; Sokka (2001) J Rheumatol, 28(7):1718-1722; Clarke (2001) J Rheumatol, 28(11):2416-2424). To evaluate the association of IL4R SNPs with disease severity, RA patients were stratified according to the radiological outcome into an erosive and a non-erosive group. Although no association between 150V and Q551R IL4R SNPs and disease susceptibility was identified, the I50V SNP was strongly associated with a fast development of joint destruction. The predictive power of I50V SNP for early erosive disease was higher than that of RF and the shared epitope (SE), identifying the I50V IL4R SNP as a novel genetic marker in RA with a high predictive value for early joint destruction. Furthermore, we addressed the functional role of I50V SNP by analyzing the response of CD4 T cells of healthy individuals with known I50V genotypes to IL4. The cells homozygous for the V50 allele demonstrated a significantly lower responsiveness to IL4 as compared to those homozygous for 150 with regard to the inhibitory effect of IL4 on Th1 cell differentiation, providing thereby a possible mechanism which might underlie the newly identified association of I50V IL4R SNP with early erosions in RA.

### Patients, Materials and Methods Used in the Present Study

Study population and clinical evaluation. 471 patients with an established diagnosis of RA according to the 1987 revised criteria of the American College of Rheumatology (Arnett (1988) Arthritis Rheum, 31:315-324) for the diagnosis of the disease were enrolled in the study (see following Table 1).

TABLE 1
Study population

| | Controls | RA patients |
|---|---|---|
| No. | 371 | 471 |
| Sex (f/m) | 264/107 | 369/102 |
| Age^{a} (yr ± SD) | 55.5 ± 16.8 | 56.9 ± 13.4 |
| Rheumatoid factor positive | n.d. | 353 |
| Shared epitope positive (1xSE/2xSE) | n.d. | 223/86 |

^{a}Age was calculated at the time of the analysis
n.d., not determined

Patients with other autoimmune connective tissue diseases such as psoriasis/psoriatic arthritis, systemic lupus erytematosis or ankylosing spondylitis were excluded from the study. The patient cohort consisted of 283 patients recruited by three rheumatologists from one of the major private rheumatology practices in Central Frankonia, a region with about 300.000 inhabitants, and of 188 patients recruited at the Department of Rheumatology at the University of Erlangen-Nuremberg and the Rheumatology Clinic Ratingen. Clinical data with potential diagnostic value such as age, sex, disease duration (DD), erythrocyte sedimentation rate (ESR), levels of C-reactive protein (CRP) and levels of IgM RF were provided by the physicians. Radiographs two years after disease onset were available from 302 patients. Two independent experienced rheumatologists who were unaware of the study questions assessed the presence or absence of erosive damage. Patients were deemed to have an erosive arthropathy if one or more definitive erosions were apparent on any peripheral joint radiograph. Patients were then stratified at two-year terms into two groups: erosive RA and non-erosive RA. A cohort of 371 healthy individuals matched on the basis of age, sex and origin (Table 1) were used as a healthy control group to evaluate the susceptibility potential of IL4R SNPs. All protocols and recruitment sites have been approved by the local institutional review board, and all subjects were enrolled with informed written consent.

DNA isolation and genotyping. 10 ml of heparinized peripheral blood were obtained from the RA patients and healthy individuals. Genomic DNA was isolated from white blood cells after the lysis of red blood cells by incubating the blood samples three times with 10 ml lysis buffer (10 mM Tris-HCl, pH 7.4, 10 mM NaCl, 3 mM MgCl₂) for 10 min on ice. Afterwards, DNA was isolated using the AquaPure Genomic DNA isolation kit (BioRad Laboratories, Munich, Germany), dissolved in water and stored at −80° C. until analysis.

Genotypes for I50V and Q551R SNPs of IL4R were determined by allele specific PCR using the Assays-by-Design service for SNP genotyping from Applied Biosystems (Darmstadt, Germany). PCR primer and probe sequences for the I50V SNP were: 5′-ACCCAGCCCCTGTGTCT-3′ (SEQ ID NO: 7) (forward primer), 5′-CGCGCCTCCGTTGTTC-3′ (SEQ ID NO: 8) (reverse primer), 5′-ACACGTGTATCCCTG-3′ (SEQ ID NO: 5) (VIC-labeled 150 allele-detecting probe) and 5′-CACGTGTGTCCCTG-3′ (SEQ ID NO: 6) (FAM-labeled V50 allele-detecting probe). PCR primer and probe sequences for the Q551R SNP were: 5′ GCCGAAATGTCCTCCAGCAT-3′ (SEQ ID NO: 9) (forward primer), 5′-TGCTCCACCGCATGTACAA-3′ (SEQ ID NO: 10) (reverse primer), 5′-CAGTGGCTATCAGGAGT-3′ (SEQ ID NO: 11) (VIC-labeled Q551 allele-detecting probe) and 5′-CAGTGGCTATCGGGAGT-3′ (SEQ ID NO: 12) (FAM-labeled R551 allele-detecting probe). PCR was performed using the TaqMan Universal PCR Master Mix (Applied Biosystems) in the ABI PRISM 7000 Sequence Detection System (Applied Biosystems).

To determine shared epitope (SE) homo- or heterozygosity, HLA-DRB1 typing was performed using the DRB high-resolution kit from Biotest (Dreieich, Germany) as instructed by the manufacturer in the HLA-typing laboratory of the University of Erlangen.

Statistical analysis. Odds ratios (ORs), p values, sensitivities and specificities were calculated for each individual analysis, along with 95% confidence intervals (95% CIs) by use of standard contingency tables (or two-tailed Fisher's exact test). Additional analyses were performed to account for individual matching between different RA patient groups using logistic regression (LR) as described elsewhere (Breslow (1980) IARC Sci Publ (32):5-338). To assess the input of HLA-DRB1 genotypes, samples were binned according to the number of SE alleles: high risk, two SE alleles; intermediate risk, one SE allele; low risk, no SE allele (Gorman (2004) Arthritis Rheum, 50(2):400-412). Hardy-Weinberg equilibrium for the distribution of genotypes was assessed by Fisher's exact test. Haplotype frequencies were estimated using the expectation maximization algorithm as described elsewhere (Excoffier (1995) Mol Biol Evol, 12(5):921-927). Results of experiments addressing the functional consequences of the IL4R SNPs were analyzed by one-way analysis of variance followed by the Bonferroni test.

Antibodies and cytokines. The following monoclonal antibodies (mAb) and recombinant cytokines were used for cell purification, culture and staining: anti-CD3 (OKT3), anti-CD8 (OKT8), anti-CD45RO (UCHL1), anti-CD16 (3g8FcIII; all from the American Type Culture Collection); anti-CD19 (Dako Diagnostika, Hamburg, Germany); anti-human CD28 (28.2; BD PharMingen, Heidelberg, Germany); neutralizing anti-human IL4 and human recombinant IL4 (both from Endogen, Woburn, Mass.); human recombinant IL2 (Chiron, Munich, Germany); FITC- or PE-conjugated mAbs to CD3, CD4, CD45RA (all from Dako Diagnostika); PE-labeled anti-IL4 (MP4-25D2), PE-labeled anti-IL2 (MQ1-17H12), FITC-conjugated anti-IFNγ (4S.B3), and PE-conjugated anti-IL12Rβ2 (BD Pharmingen). The isotype-matched control for IL12Rβ2, PE-conjugated rat IgG2a, was purchased from BD Pharmingen.

Cell purification. Total CD4 T cells were isolated from heparinized peripheral blood of healthy individuals with identified IL4R genotypes by gradient centrifugation over a Ficoll layer (Biotest) followed by rosetting of mononuclear cells with sheep erythrocytes and negative selection of CD4 T cells from the rosette positive fraction using mAbs against CD8, CD19, and CD16. For the purification of naive CD4 T cells, an additional panning with mAbs to CD45RO was performed as previously described (Skapenko (2001) J Immunol, 166(7):4283-4292). The purity of the recovered cell population was assessed by flow cytometry. Typically, ≧90% of the cells were positive for CD3 and CD4, ≧90% of isolated naive T cells were positive for CD45RA, and ≧98% of the cells were viable after purification.

Cell culture. Cell cultures were carried out in RPMI 1640 medium supplemented with penicillin G/streptomycin (50 U/ml), L-glutamine (2 mM) (all from Gibco-Invitrogen, Karlsruhe, Germany), and 10% normal human serum. T cell differentiation was assessed employing an in vitro, multiple-step differentiation system as previously described (Schulze-Koops (1998) Eur J Immunol, 28:2517-2529; Skapenko (2001) loc. cit.). In brief, freshly isolated naive CD4 T cells (0.5×10⁶/ml) were primed by plate-bound OKT3 (1 μg/ml) and soluble anti-CD28 mAbs (1 μg/ml) in the presence of IL2 (10 U/ml) and in the presence of either anti-IL4 (10 μg/ml) or IL4 (31.25 ng/ml). After 5 days of priming, the cells were harvested, washed, counted, and rested for 2.5 days at a concentration of 1×10⁶ cells/ml in medium supplemented with IL2 (10 U/ml). The differentiation of the T cells was determined by intracellular flow cytometry of cytoplasmic cytokines. To analyze the IL12Rβ2 chain expression, freshly isolated CD4 T cells were activated with immobilized OKT3 (1 μg/ml) and soluble anti-CD28 mAbs (1 μg/ml) in the presence of IL2 (10 U/ml) and either anti-IL4 (10 μg/ml) or IL4 (31.25 ng/ml for IL12Rβ2 protein or 7.8 ng/ml for IL12Rβ2 mRNA expression analysis). IL12Rβ2 protein surface expression was assessed after 96 h of culture by flow cytometry. IL12Rβ2 mRNA levels were determined by quantitative RT-PCR after 48 h of priming.

Flow cytometry. To determine the phenotype of differentiated CD4 T cells, the cells were stimulated with ionomycin (1 μM, Calbiochem, Schwalbach, Germany) and PMA (20 ng/ml, Sigma-Aldrich, Taufkirchen, Germany) for 5 h at 37° C. in the presence of 2 μM monensin, harvested, washed with PBS (Gibco Invitrogen) and fixed with 3% paraformaldehyde (Sigma-Aldrich) in PBS for 10 min at 37° C. Cells were permeabilized with 0.1% saponin (Sigma-Aldrich) in 2% FCS/PBS, and the unspecific binding sites were blocked with 4% rat and mouse serum (both from Sigma-Aldrich). Cells were incubated for 30 min at 4° C. with saturating amounts of directly fluorochrome-labeled mAbs against IL2, IL4, and IFNγ. After washing with 0.1% saponin in 2% FCS/PBS, cells were resuspended in 2% FCS/PBS and analyzed by flow cytometry (Skapenko (1999) J Immunol, 163:491-499). For measuring IL12Rβ2 expression on the surface of activated cells, 100×10³ cells were washed with 2% FCS/PBS, incubated with saturating amounts of PE-labeled Abs to IL12Rβ2 for 15 min at 4° C. and analyzed by flow cytometry.

Total RNA isolation and quantitative RT-PCR. CD4 T cells were activated as described above, harvested, washed with PBS, and total RNA was isolated using the RNeasy Mini Kit (Qiagen, Hilden, Germany). To exclude genomic DNA contamination, RNA samples were treated with DNase (Qiagen). mRNA was transcribed to cDNA for 1 h at 42° C. in a total volume of 20 μl containing 1×AMV RT buffer (Promega, Mannheim, Germany), 1 mM dNTPs, 100 ng/ml oligo dT₁₂₋₁₈ (both from Amersham Pharmacia Biotech, Freiburg, Germany), and 0.25 U/μl AMV RT (Promega). Real-time PCR was performed in duplicates using the Universal PCR Master Mix in the ABI Prism 7000 Sequence Detection System (all from Applied Biosystem, Darmstadt, Germany). Assays-on-Demand Gene Expression Products for cyclophilin from Applied Biosystem was used to determine cyclophilin mRNA expression. To determine IL12Rβ2 mRNA expression, a set of primers consisting of 5′-TCCAGATCCAGCAAATAGCACTTG-3′ (SEQ ID NO: 13) (forward primer), 5′-AGTCTATCAGGAGCCTGTCCAA-3′ (SEQ ID NO: 14) (reverse primer) and 5′-TTGCAGAGGAGAAGACAC-3′ (SEQ ID NO: 15) (FAM-labeled probe) was designed by the Custom TaqMan Gene Expression Assay Service (Applied Biosystems). Relative quantification of IL12Rβ2 cDNA to cyclophilin was determined by calculating the difference in cross-threshold values (ΔCt) according to the formula 2^{-ΔCt}.

### Example 1

### Patient Characterization; Incidence/Susceptibility and Association with Destruction Disease

The 471 patients represent a geographically diverse, ethnically homogeneous cohort of RA patients who are likely to be broadly representative of patients with RA seen by rheumatologists (see Table 1 above). The mean±SD age of the patients at time of the analysis was 56.9±13.4 years, and 78% were female. 353 patients (75%) were RF positive, and 309 patients (66%) were positive for at least one SE allele. The mean CRP±SEM value was 19.9±0.2 mg/l, and the mean±SEM ESR was 27.8±1.2 mm/h. At the time of analysis mean±SD DD was 7.0±6.1 years. Radiographs of the hands and/or feet two years after disease onset were available from 302 patients.

In order to examine the possible contribution of I50V and Q551R polymorphisms of IL4R to RA susceptibility, a case-control association study was performed. The distribution of the genotypes in RA patient and in control groups is shown in Table 2.

TABLE 2
Frequency (%) of IL4R genotypes in individuals with RA and controls

| | Controls | RA patients | |
|---|---|---|---|
| Genotype | (n = 371) | (n = 471) | p^{a} |
| At nt 148 (I50V) | | | |
| AA (I50I) | 29.9 (n = 111) | 28.5 (n = 134) | — |
| AG (I50V) | 46.9 (n = 174) | 51.4 (n = 242) | 0.42 |
| GG (V50V) | 23.2 (n = 86) | 20.2 (n = 95) | 0.69 |
| At nt 1652 (Q551R) | | | |
| AA (Q551Q) | 61.2 (n = 227) | 64.5 (n = 304) | — |
| AG (Q551R) | 35.3 (n = 131) | 30.8 (n = 145) | 0.21 |
| GG (R551R) | 3.5 (n = 13) | 4.7 (n = 22) | 0.60 |

^{a}Values were calculated based on the individual numbers by use of Fisher's exact test

Each polymorphism was in Hardy-Weinberg equilibrium, and the frequencies were consistent with those previously reported by others (Hackstein (1999) Hum Immunol, 60(11):1119-1127, Howard (2002) Am J Hum Genet, 70(1):230-6). There were no significant differences in the genotype and allele frequencies between RA patients and healthy controls. Moreover, no differences were observed between RA patient and control cohorts in the distribution of possible IL4R haplotypes calculated based on the frequencies of those two SNPs either (data not shown). Thus, IL4R seems not to be associated with RA susceptibility.

To evaluate the impact of IL4R on disease development, we classified the RA patients, from whom radiographs of the hands and/or feet were available two years after disease onset into two groups with erosive and non-erosive disease, respectively. 151 patients showed joint erosions in at least one joint typically affected in RA and 151 patients had no signs of bone destruction at two years DD. Whereas a significant difference in the distribution of the I50V SNP genotypes was observed in the patients with erosive compared to those with non-erosive disease (χ² 15.68, p=0.0004; FIG. 1A, left panel), the distribution of genotypes for the Q551R SNP was similar in both groups (χ² 0.11, p=0.95; FIG. 1A, right panel). Epidemiologic characteristics of the three groups of patients homo- and heterozygous for the I50V SNP are summarized in Table 3 below.

TABLE 3
Epidemiologic parameters of the patients with available radiographs
two years after RA onset

| | Total cohort | AA (I50I) | AG (I50V) | GG (V50V) |
|---|---|---|---|---|
| No. | 302 | 92 | 138 | 72 |
| Sex (f/m) | 229/73 | 66/26 | 104/34 | 59/12 |
| Age^{a} (yr ± SD) | 54.4 ± 13.7 | 53.3 ± 13.9 | 55.9 ± 12.9 | 52.8 ± 14.8 |
| Rheumatoid | 220 | 65 | 98 | 57 |
| factor positive | | | | |
| Shared epitope | 144/57 | 41/18 | 64/28 | 39/11 |
| positive | | | | |
| (1xSE/2xSE) | | | | |

^{a}Age was calculated at the time point of two years after disease onset

No significant differences were observed between the three groups and between any of the groups and the total patient cohort either. Moreover, the epidemiologic characteristics of all the three groups were representative of those from the total cohort. Nevertheless, patients homozygous for the V50 mutation developed significantly more often erosive disease within the first two years of the disease than those homozygous for 150 (OR 3.63, 95% CI 1.89-6.97, p<0.0001; Table 4). 68.1% of the V50 compared to only 37.0% of the 150 homozygous patients showed radiographic erosions at two years DD. The association was independent of individual factors previously associated with severe disease, as CLRs that adjusted for either SE or RF revealed no significant changes in the OR (OR adjusted for SE 3.52, 95% CI 1.84-6.75, p<0.0001; OR adjusted for RF 3.50, 95% CI 1.83-6.69, p<0.0001). Adjusting for HLA-DRB1 genotypes, RF, sex and age resulted even in a slight increase of the OR to 3.86 (95% CI 1.95-7.64, p<0.0001; see following Table 4). Patients heterozygous for I50V SNP had also an almost two times higher risk to develop joint destruction already in the first two years after disease onset (OR 1.69, 95% CI 0.96-2.98; see following Table 4).

TABLE 4
Association of IL4R with bone erosion in RA

| | | | Results for | | | | | |
|---|---|---|---|---|---|---|---|---|
| | Erosive disease^{a} | | Crude analysis^{b} | | | PCLR^{d} | | |
| Genotype | Yes | No | OR^{c} | 95% CI | p | OR^{c} | 95% CI | p |
| AA (I50I) | 34 | 58 | — | — | — | — | — | — |
| AG | 68 | 70 | 1.66 | 0.97-2.84 | 0.08 | 1.69 | 0.96-2.98 | 0.07 |
| (I50V) | | | | | | | | |
| GG | 49 | 23 | 3.63 | 1.89-6.97 | <0.0001 | 3.86 | 1.95-7.64 | <0.0001 |
| (V50V) | | | | | | | | |

^{a}Joint destruction within the first 2 years of the disease was determined by X-ray analysis
^{b}Values were calculated by use of Fisher's exact test
^{c}ORs were calculated relative to the reference allele homozygote (AA)
^{d}CLR was performed to account for individual matching for sex, age, RF, and SE between different RA patient groups

The contribution of I50V IL4R SNP to the development of erosive disease demonstrates, therefore, a significant allele-dose effect of V50 (χ² for trend 15.37, p<0.0001) and can be illustrated by the increase of patients with erosive disease with increasing numbers of the V50 allele (FIG. 1B). These data indicate that the presence of the V50 allele favors accelerated destruction of the joints during early RA independent of the HLA-DRB1 genotype and of RF.

### Example 2

### The Predictive Value of the V50 Allele

The increased risk of RA patients carrying a V50 allele to develop early erosive disease implicates a potential predictive value of IL4R in RA. Evaluation of the sensitivity and the specificity of the V50 allele for early development of joint erosion revealed that they both were comparable to those of the SE or the RF; see Table 5 below.

TABLE 5
Sensitivity and specificity of IL4R as a predictive marker
for early bone erosion in RA^{a}

| | Sensitivity | Specificity |
|---|---|---|
| Predictive parameter | (95% CI) | (95% CI) |
| 1x IL4R/V50^{b} | 0.78 (0.70-0.84) | 0.38 (0.31-0.47) |
| 2x IL4R/V50 | 0.33 (0.25-0.41) | 0.85 (0.78-0.90) |
| 1x IL4R/V50 + HLA-DRB1/SE^{c} | 0.58 (0.49-0.66) | 0.64 (0.55-0.71) |
| 2x IL4R/V50 + HLA-DRB1/SE | 0.23 (0.17-0.31) | 0.90 (0.84-0.94) |
| 1x IL4R/V50 + RF | 0.56 (0.49-0.65) | 0.54 (0.46-0.62) |
| 2x IL4R/V50 + RF | 0.27 (0.20-0.35) | 0.89 (0.83-0.94) |
| 1x IL4R/V50 + HLA-DRB1/SE + | 0.47 (0.39-0.55) | 0.70 (0.62-0.77) |
| RF | | |
| 2x IL4R/V50 + HLA DRB1/SE + | 0.21 (0.15-0.28) | 0.92 (0.87-0.96) |
| RF | | |
| HLA-DRB1/SE | 0.72 (0.64-0.79) | 0.39 (0.31-0.47) |
| RF | 0.73 (0.65-0.80) | 0.27 (0.20-0.35) |
| HLA-DRB1/SE + RF | 0.60 (0.53-0.68) | 0.49 (0.41-0.57) |

^{a}Values were calculated based on the individual numbers by use of Fisher's exact test
^{b}IL4R/V50, V50 allele of IL4R
^{c}HLA-DRB1/SE, HLA-DRB1 alleles that share the SE sequence

The presence of two V50 alleles increased the specificity of IL4R to 0.85. Moreover, in combination with either other investigated predictive marker, SE or RF, the specificity of the presence of two V50 alleles was further increased and, if SE and RF were both present, the sensitivity was as high as 0.92. In marked contrast, the combination of SE and RF without the impact of IL4R did not reach even the predictive power of V50 homozygosity alone. Thus, IL4R appears to be a novel genetic marker with high predictive value for early erosive RA and might therefore be of significance in the clinic to identify patients with accelerated joint destruction early before the development of rheumatoid damage.

### Example 3

### Elucidation of Biological Mechanisms and Interpretation of Results

Based on the observation that an exaggerated Th1 cell differentiation is associated with accelerated disease progression (Kanik (1998) loc. cit.; van der Graaff (1998) loc. cit.) and on the role of IL4 in controlling Th1 differentiation, we carried out experiments to test whether IL4R SNPs can modulate the inhibitory effect of IL4 on Th1 differentiation in healthy individuals with a known I50V SNP genotype. Naive CD4 T cells were primed in vitro for 5 days with anti-CD3 and anti-CD28 antibodies to induce Th1 cell differentiation. Neutralizing anti-IL4 antibodies or IL4 were added to the cultures to allow the assessment of the inhibitory effect of IL4. After additional 2.5 days of resting, the extent of Th1 cell differentiation was estimated by flow cytometry of cytoplasmic IFNγ and IL4 (FIGS. 2A, B). As shown in FIG. 2C (left panel), Th1 cell differentiation that occurred in the presence of neutralizing anti-IL4 antibodies was comparable in all three individual groups. The presence of IL4 in the culture resulted in a significant reduction in the frequencies of IFNγ-producing Th1 cells in all groups. Strikingly, however, when the extent of Th1 cell differentiation in the presence of IL4 was normalized to the respective value of Th1 differentiation in the presence of anti-IL4, a clear difference in the effect of IL4 between the three groups became apparent (FIG. 2C, right panel). In other words, the extent of the inhibition of Th1 cell differentiation by IL4 was significantly less pronounced in the group of individuals homozygous for the variant allele, V50 as compared to the groups of I50I or I50V individuals (FIG. 2C, right panel).

In a similar way, we investigated the inhibitory effect of IL4 on mRNA and protein expression of IL12Rβ2, a subunit of the IL12R expressed on developing Th1 cells (Szabo (1997) J Exp Med, 185:817-824) that facilitates the sensitivity of the cells to the Th1-inducing activity of IL12 (FIG. 3A). Whereas both mRNA and protein expression of IL12Rβ2 were significantly reduced in CD4 cells that were cultured in the presence of IL4 as compared to those cultured with neutralizing anti-IL4 antibodies independent of the genotype group (FIG. 3B, 3C, left panels), normalized expression levels demonstrate significant differences between the groups (FIG. 3B, 3C, right panels). The extent of IL4 inhibition for both, protein and mRNA levels of IL12β2 was about two times as pronounced in T cells from 1501 individuals compared to T cells isolated from V50V individuals. Together, V50 homozygosity mediated a markedly reduced response of the T cells to IL4. This might contribute to a more intensive Th1 cell differentiation in patients homozygous for V50 and lead in those patients to accelerated erosive disease.

The results provided alone are clear evidence for an association of the V50 allele of IL4R with the fast development of radiographic bone erosions in the joints of patients with RA. This is surprising in the light of previously published evidence that the I50V allele/SNP has no predictive value and is not informative; see Goronzy (2004), loc. cit. While the association of one V50 allele of the IL4R with erosive disease was relatively modest (yet informative), it was strong with an OR as high as 3.86 (95% CI 1.95-7.64) if two V50 alleles were present. The data provided here show the first association of the V50 allele of IL-R4 with erosive disease in RA.

Although a majority of previous studies demonstrate an association of the SE sequence-sharing HLA-DRB1 alleles with the development of erosive disease, some striking inconsistencies exist (Meyer (1999) J Rheumatol, 26(5):1024-1034; Combe (1995) Br J Rheumatol, 34(6):529-534; Thomson (1999) loc. cit.; Gorman (2004) loc. cit.), indicating that other genetic loci influencing disease progression might exist. Identification of reliable predictors of severity early in the disease is important, on the other hand, because of the correlation of erosions with clinical outcome such as disability (Kirwan (2001) loc. cit.; Sokka (2001) loc. cit.) and the recent demonstration that early treatment may retard bone destruction (O'Dell (2002) loc. cit.). The development of anti-tumor necrosis factor agents such as etanercept (Bathon (2000) N Engl J Med, 343(22):1586-1593) and infliximab (Lipsky (2000) N Engl J Med 2000; 343(22):1594-1602) has revolutionized the treatment of RA, although at this time, long-term safety information is missing and TNF-neutralizing treatment is extremely expensive. Therefore, the potential to more precisely predict early in the disease which individual will require more aggressive treatment would be especially valuable.

The association of the I50V SNP (or “I75V” SNP) with bone erosions of RA patients already at two years DD suggests a potential of this polymorphism as a predictive marker. In fact, the predictive value estimate of the V50 allele revealed the powerful capacity of this allele to identify individuals with a high risk of early onset of joint destruction. Although typing of the patients just for the presence of the V50 allele demonstrated relatively low specificity, identification of patients homozygous for the V50 allele allows the prediction of a fast destructive course of the disease with reasonable confidence (specificity at 0.85). The predictive value of one V50 allele alone in our cohort of the RA patients was comparable to that of the RF or of one SE allele. The ORs of RF as well as SE alleles observed in our study, on the other hand, were comparable to those reported by others (Thomson, (1999) loc. cit.; Meyer (2003) Ann Rheum Dis, 62(2):120-126; Gorman (2004) loc. cit.). However, in contrast to RF or to SE alleles, the combination of the V50 allele with either other predictive marker resulted in an increase of the predictive power to over 0.50, whereas the combination of RF and SE alleles even did not reach that value. Moreover, the predictive value of the RF and SE alleles in combination was much lower than the predictive value of V50 homozygosity, emphasizing the powerful capacity of IL4R as a newly identified predictive parameter to identify at-risk RA patients early in the disease.

Elucidating the genetic basis of RA is currently one of the major challenges in modern rheumatology. RA is a complex autoimmune disease that is, unlike those Mendelian traits causally related to the highly penetrant rare mutations of single-genes, caused by small individual effects of many low penetrant common alleles. This makes the identification of the autoimmune disease-related genes substantially difficult. As documented herein the V50 allele of IL4R is a reliable marker for susceptibility of RA because of several reasons: 1) its chromosomal position, 2) the important role of its ligand, IL4, in the pathogenesis of the disease, 3) previous identification of SNPs leading to functional alterations of IL4R, and 4) associations of IL4R with other complex diseases characterized by a Th1/Th2 shift. Therefore, we undertook a case-control association study with 471 RA patients and 371 age- and sex-matched healthy individuals. However, no association between the IL4R and RA susceptibility was detected in our cohort of RA patients. In our analysis, though, we focused only on those SNPs, which were previously demonstrated to lead to some functional alterations of the IL4R. An additional analysis of non-synonymous SNPs, or SNPs in the non-coding region of IL4R, or synonymous SNPs that, however, did not result in detectable functional alterations in cellular assays might be required to fully exploit the association of IL4R with RA susceptibility. Initial investigations of IL4R SNPs in case-control studies revealed association of the two individual SNPs, I50V (Mitsuyasu (1998) loc. cit.) and Q551R (Hershey (1997) loc. cit.) with atopic disorders. In contrast, a later investigation analyzing multicase atopic families was able to identify a significant association of atopy with IL4R only if seven IL4R SNPs were determined simultaneously, indicating an association of atopic disorders with particular haplotypes of IL4R (Ober (2000) Am J Hum Genet, 66(2):517-526). Likewise, only a defined haplotype of IL4R could be associated with diabetes in a study of diabetes multiplex families with affected siblings pairs (Mirel (2002) loc. cit.). Even more, only a particular combination of SNPs in IL4R and in another relevant gene, for example IL13, has been demonstrated to be associated with diabetes among Filipinos or with asthma in a Dutch population (Bugawan (2003) loc. cit.; Howard (2002) Am J Hum Genet, 70(1):230-236). All together, these data confirm the difficulties in exploitation of the genetic basis of complex diseases and indicate the necessity to expand the number of investigated SNPs involving even those SNPs that are located in genes others than IL4R.

Because of the well-established linkage between IL4R and atopic disorders (Mitsuyasu (1998) loc. cit.; Hershey (1997) loc. cit.; Ober (2000) loc. cit.) several investigations have been performed in attempts to identify whether IL4R allelic variants may lead to alterations in cell functions. Specific signaling of the IL4R is transmitted mainly via signal transducer and activator of transcription 6 (STAT6) (Nelms (1999) Annu Rev Immunol, 17:701-738). A broad spectrum of genes is transcriptionally regulated via STAT6. STAT6 facilitates the expression of IgE and its receptor, CD23, in B cells and of a number of genes involved in Th2 differentiation in CD4 T cells. On the other hand, STAT6 acts as a repressor of the transcription of genes involved in Th1 cell differentiation, such as IFNγ, IL12Rβ2, and IL18Rα. Despite of the I50V SNP vicinity to the IL4-binding site of the IL4R no alterations in IL4R affinity to IL4 have been observed (Mitsuyasu (1998) loc. cit.; Mitsuyasu (1999) loc. cit.). Nevertheless, a more pronounced STAT6-dependent transcriptional activity in Jurkat T cells transfected with the 150 allelic variant of IL4R has been detected (Stephenson (2004) J Immunol, 173(7):4523-4528). Accordingly, a more pronounced response to IL4 of B cells bearing the 150 variant compared to those bearing the V50 variant with regard to CD23 expression and IgE synthesis has been reported (Mitsuyasu (1999) loc. cit.). We found that CD4 T cells isolated from individuals homozygous for the 150 allele demonstrated significantly better responses to IL4 compared to those CD4 T cells isolated from V50V individuals. I50I CD4 T cells reacted more effectively to the inhibitory effect of IL4 on Th1 cell differentiation. Our observation that the inhibitory effect of IL4 on Th1 cell differentiation can be modulated by IL4R allelic variants appears, however, is in contrast to a recent publication (Stephenson (2004) loc. cit.). Without being bound by theory, this may be explained by the fact that in that report murine CD4 T cells transfected with different IL4R constructs were used, whereas we worked with primary CD4 T cells freshly isolated from individuals with determined I50V genotype. The less pronounced inhibition of Th1 cell differentiation as well as of IL12Rβ2 expression by IL4 in CD4 T cells from V50V individuals as demonstrated here fits well with the association of the V50 homozygosity with accelerated radiographic joint erosions, since it is conceivable that a less controlled Th1-mediated rheumatic inflammation would drive more aggressively destructive joint processes. Herein a mechanism is proposed which might underlie the association of V50 SNP of the IL4R with rapid development of erosions in RA, implicating thereby a functional impact of the IL4R in RA pathophysiology. Yet, it is to be understood that the present invention is not limited to the herein described and proposed mechanism.

In this invention we investigated the association of IL4R with both, disease susceptibility and disease progression. Although no association was observed with the onset of the RA, a strong linkage between the I50V IL4R SNP and rapid rheumatic joint destruction became apparent. The predictive value of the I50V IL4R SNP for the development of erosive disease was superior to that of conventional biomarkers of joint destruction, RF and the presence of the SE. As a possible mechanisms underlying this association, it could be shown that the mutant V50 allele confers reduced responsiveness of T cells to the immunomodulatory effects of IL4, resulting in decreased down-modulation of the IL12β2R and reduced inhibition of Th1 cell differentiation. The data provided herein identify a unique genetic marker with high predictive value that is to be used for the benefit of RA patients to identify those with erosive disease early before irreversible joint damage has occurred. According therapeutic strategies can then be developed in accordance with the genetic pre-disposition of the individual.

### Example 4

### Specificity of the IL4R Single-Nucleotide Polymorphism I50V as a Predictive Marker in Rheumatoid Arthritis: No association with Erosive Disease in Psoriatic Arthritis

IL4 receptor polymorphisms have been demonstrated to predispose to an imbalance in the Th1/Th2 ratio of adaptive immune responses in a variety of immune disorders (Romagnani, Immunol Today (1997) 18:263-6). Moreover, association studies as provided in the above examples of two functional single nucleotide polymorphisms in the coding region of the IL-4 R gene, the I50V and the Q551R polymorphisms (which are on different haplotype blocks) has demonstrated that the inheritance of the 50V encoding SNP enhances the genetic risk for joint destruction and an erosive Th1-biased disease course in rheumatoid arthritis considerably. Since the imbalance of Th1/Th2 immune responses has also been suggested to play an important role in psoriasis (Schlaak, J Invest Dermatol. (1994) 102:145-9) and psoriatic arthritis (Ritchlin, J. Rheumatol. (1998) 25:1544-52; Partsch, Ann Rheum Dis. (1998) 57: 691-3), we have decided to perform an association study of the I50V and Q551R polymorphism in large cohorts of German patients suffering from psoriasis either with or without arthritic manifestations.

The 375 single patients with psoriasis vulgaris (Huffmeier , J Invest Dermatol. (2005) 125: 906-12) were recruited through dermatology clinics at two psoriasis rehabilitation hospitals. An early onset form of psoriasis was diagnosed in all but three patients with an average age of onset of 23±11 years. The majority of patients suffered from plaque type of psoriasis vulgaris. 62% of patients were male. The study including recruitment of controls was approved by the ethical committee of the University of Münster.

The 375 patients (Lascorz, Ann Rheum Dis. (2005) 64: 951-4) with psoriatic arthritis were similarly of German descent and recruited through four different rheumatological centres in Germany: one acute clinic, two rehabilitation hospitals and one private practice. The diagnosis of PsA was made by a board certified rheumatologist according to the criteria of Moll and Wright (Semin Arthritis Rheum 1973; 3: 55-78) in addition all patients included into the investigation also fulfilled the recently developed CASPAR criteria (ClASsification of Psoriatic ARthritis study group). Patients exhibiting erosions in at least one joint on plain radiographs of hand and feet or any other joint investigated were regarded as to have erosive arthritis. Patients without any signs of joint erosion were assigned to have a non-erosive disease course. The study was approved by the Ethics Committee of the Friedrich-Alexander-University of Erlangen-Nuremberg.

Average age of onset for psoriasis vulgaris was 30±13 years. 59.9% of patients were male. All patients were negative for the rheumatoid factor. For 78% of the patients, the diagnosis of psoriatic arthritis was made ≧3 years prior to recruitment. Erosive disease was recorded in 197 patients (53%). In 178 patients (47.5%) the erosive disease was associated with a disease duration of more than 3 years. PsA persisting for a a period longer than three years as a non-erosive disease was recorded in 79 patients (20.8%). 19 patients with erosive and 36 patients with non-erosive arthritis were not included in the association study on erosive outcome due to the limited arthritis duration of less than 3 years, while for 63 patients (17%) no data about the radiographic status were available.

Controls. The 376 controls had no psoriasis vulgaris at the time of recruitment when average age was 32±10 years. All of them were healthy blood donors and recruited in Northern Germany. 59% of probands were male.

All patients and controls were of German origin. The individuals gave their written informed consent. The investigations were conducted according to Declaration of Helsinki principles.

Genotyping of the two SNPs in the IL4R gene was performed with either Taqman SNP Genotyping Assays from Applied Biosystems (Foster City, USA) in the case of rs1805010 (I50V) or with self-designed primers and TaqMan mgb-probes for rs1801275 (Q551R). All Taqman reactions were carried out in an ABI Prism 7900HT Sequence Detection System (Applied Biosystems) with 10 ng of genomic DNA as a template in a 5 μl reaction using standard thermal cycling conditions. Sequences of primers and probes as well as assay IDs are available upon request. Taqman genotypes for both polymorphisms were verified by direct sequencing of 24 randomly chosen samples.

The analysis did not reveal significant differences in allele frequencies between controls and the cohorts of PsA or psoriasis vulgaris patients for any of the two IL-4R variants under investigation, see following table.

| | | Psoriasis vulgaris | | | Controls | Psoriatic arthritis | | |
|---|---|---|---|---|---|---|---|---|
| Polymorphism | | n (%) | χ² | p-value | n (%) | n (%) | χ² | p-value |
| rs1805010 | Allele A | 402 (55) | 0.017 | 0.896 | 405 (55) | 415 (57) | 0.669 | 0.413 |
| (I150V) | Allele G | 328 (45) | | | 335 (45) | 315 (43) | | |
| rs1801275 | Allele A | 588 (80) | 0.033 | 0.856 | 582 (79) | 587 (79) | 0.0 | 1.0 |
| (Q551R) | Allele G | 150 (20) | | | 152 (21) | 153 (21) | | |

Allele frequencies of the two polymorphisms in IL4R in patients and controls and results of the χ² statistics.

Hardy-Weinberg equilibrium for both SNPs was confirmed in all cohorts. By contrast to recent results on the predictive value of the 50V variant of the IL-4 receptor for a more rapid progression of bony erosions in RA our stratification of the PsA cohort according to erosive or non-erosive outcome after a disease course of at least 3 years did not show association to the analyzed two functional SNP polymorphism, see the following table:

| | | PsA erosive | | | PsA non- |
|---|---|---|---|---|---|
| Polymorphism | | n (%) | χ² | p-value | erosive n (%) |
| rs1805010 | Allele A | 200 (57) | 0.152 | 0.696 | 84 (55) |
| (I50V) | Allele G | 150 (43) | | | 68 (45) |
| rs1801275 | Allele A | 283 (80) | 0.161 | 0.688 | 123 (79) |
| (Q551R) | Allele G | 69 (20) | | | 33 (21) |

Allele frequencies of the two polymorphisms in IL4R in the PsA patients with and without erosive arthritis after a disease course >3 years and results of the χ² statistics.

This lack of association did not change upon the consideration of an allele dosage effect in a slightly modified statistical analysis, see the following table:

| Genotype | | | | |
|---|---|---|---|---|
| rs1805010 (I50V) | Yes | No | OR^{§} | P^{†} |
| AA (I) | 54 | 21 | | |
| AG | 92 | 42 | 0.85 | 0.64 |
| GG (V) | 29 | 13 | 0.86 | 0.83 |

^{†}Values were calculated with Fisher's exact test.
^{§}Odds ratios (ORs) were calculated relative to the reference allele homozygote (AA).

Erosive disease was determined by analysis of radiographs.

Thus also homozygosity for the IL4R 50V allele that is a risk for an erosive RA course did not prove to have the same predictive value for radiographic outcome in PsA. Particularly interesting are two aspects of these results. 1. The predictive value of homozygosity for the IL4R-50V allele has been suggested to be used as a genetic marker for the decision making on therapeutic treatment strategies in early RA especially on expensive modalities such as anti-TNF-therapy that is also successfully applied to PsA. These studies suggest that this marker would not improve patient stratification according to individual risk profiles in PsA by contrast to RA where it might be beneficial. 2. Despite the data on the functional impact of the IL-4R50V variant on IL-4 cytokine-induced Stat 6 activation and subsequent modulations of cytokine receptor expression and cell differentiation, the consequences of the same polymorphism for joint pathology in RA, as shown in the example above, PsA still seem to differ considerably despite the fact that a Th1 polarized phenotype is dominating the T cell population in the joints in both diseases.

1. A method of diagnosing and/or predicting joint destruction, early joint destruction and/or accelerated joint destruction comprising determining in a sample obtained from an individual the presence of at least one nucleic acid sequence selected from the group consisting of
(a) a nucleic acid sequence encoding an IL-4 receptor (IL-4R) which contains a mutation in position 465 of the nucleotide sequence of the wild-type IL4R as shown in SEQ ID NO: 1, whereby at said position the nucleotide A is replaced;
(b) a nucleic acid sequence encoding an IL-4 receptor (IL-4R) which contains a mutation in position 465 of the nucleotide sequence of the wild-type IL4R as shown in SEQ ID NO: 1, whereby at said position the nucleotide A is replaced by the nucleotide G;
(c) a nucleic acid sequence encoding an IL-4 receptor (IL-4R), said IL-4R comprising at position 75 as shown in SEQ ID NO: 2 a valine;
(d) a nucleic acid sequence encoding an IL-4 receptor (IL-4R), said IL-4R comprising at position 75 as shown in SEQ ID NO: 2 a valine instead of an isoleucine;
(e) a nucleic acid sequence comprising at least 15 nucleotides of the nucleic acid sequence of any one of (a) to (d) and comprising the mutation as defined in any one of (a) to (d);
(f) a nucleic aid sequence comprising a nucleotide sequence as shown in any one of SEQ ID NO: 3 or a fragment of the sequence as shown in SEQ ID NO: 3, said fragment comprising the nucleotide sequence “gtc”/“guc” as shown in position 5758 to 5760 of SEQ ID NO: 3;
(g) a nucleic acid sequence encoding a polypeptide comprising the amino acid sequence as shown in SEQ ID NO: 4;
(h) a nucleic acid sequence which hybridizes to a nucleotide sequence defined in any one of (a) to (g) and having a mutation as defined in any one of (a) to (d); and
(i) a nucleic acid sequence being degenerate as a result of the genetic code to the nucleic acid sequence as defined in (h).

2. A method of diagnosing and/or predicting early joint destruction and/or accelerated joint destruction comprising determining in a sample obtained from an individual the presence of an encoded IL-4 receptor (IL-4R) which comprises at the homologous position 75 of IL-4 receptor as depicted in SEQ ID NO: 2 a mutation, said mutation comprising the exchange from an isoleucine to a valine.

3. The method of claim 1, wherein said mutation is detected in homozygous alleles/homozygosity.

4. The method of any one of claims 1 to 3, wherein said joint destruction, early joint destruction is associated with erosion(s) and/or a higher severity in the progression of rheumatoid arthritis (RA).

6. The method of any one of claims 1 to 5, said method comprising PCR-technology, ligase-chain reaction, NASBA, restriction digestion, direct sequencing, nucleic acid amplification techniques, MALDI, MALDI-TOF, hybridization techniques and/or immuno assays.

7. Use of specific probes and/or primers for the preparation of a diagnostic composition for diagnosing and/or predicting joint destruction, early joint destruction and/or accelerated joint destruction.

8. Use of specific antibody molecules or specific binding molecules for the preparation of a diagnostic composition for diagnosing and/or predicting joint destruction, early joint destruction and/or accelerated joint destruction.

9. The use of claim 7 or 8, wherein said early joint destruction is associated with early erosion(s) and/or a higher severity in the progression of rheumatoid arthritis (RA).

10. The use of claim 7 and 9, wherein said specific probe is selected from the group consisting of 5′-ACACGTGTATCCCTG-3′ (SEQ ID NO: 5) or 5′-CACGTGTGTCCCTG-3′ (SEQ ID NO: 6).

11. The use of claim 7 or 9 and 10, wherein said primer is selected from the group consisting of 5′-ACCCAGCCCCTGTGTCT-3′ (SEQ ID NO: 7) (forward primer) or 5′-CGCGCCTCCGTTGTTC-3′ (SEQ ID NO: 8) (reverse primer).

12. A kit comprising specific probes and/or primers for diagnosing and/or predicting joint destruction, early joint destruction and/or accelerated joint destruction, said probes and/or primers being capable of detecting at least one nucleic acid sequence selected from the group consisting of
(a) a nucleic acid sequence encoding an IL-4 receptor (IL-4R) which contains a mutation in position 465 of the nucleotide sequence of the wild-type IL4R as shown in SEQ ID NO: 1, whereby at said position the nucleotide A is replaced;
(b) a nucleic acid sequence encoding an IL-4 receptor (IL-4R) which contains a mutation in position 465 of the nucleotide sequence of the wild-type IL4R as shown in SEQ ID NO: 1, whereby at said position the nucleotide A is replaced by the nucleotide G;
(c) a nucleic acid sequence encoding an IL-4 receptor (IL-4R), said IL-4R comprising at position 75 as shown in SEQ ID NO: 2 a valine;
(d) a nucleic acid sequence encoding an IL-4 receptor (IL-4R), said IL-4R comprising at position 75 as shown in SEQ ID NO: 2 a valine instead of an isoleucine;
(e) a nucleic acid sequence comprising at least 15 nucleotides of the nucleic acid sequence of any one of (a) to (d) and comprising the mutation as defined in any one of (a) to (d);
(f) a nucleic aid sequence comprising a nucleotide sequence as shown in any one of SEQ ID NO: 3 or a fragment of the sequence as shown in SEQ ID NO: 3, said fragment comprising the nucleotide sequence “gtc”/“guc” as shown in position 575.8 to 5760 of SEQ ID NO: 3;
(g) a nucleic acid sequence encoding a polypeptide comprising the amino acid sequence as shown in SEQ ID NO: 4;
(h) a nucleic acid sequence which hybridizes to a nucleotide sequence defined in any one of (a) to (g) and having a mutation as defined in any one of (a) to (d); and
(i) a nucleic acid sequence being degenerate as a result of the genetic code to the nucleic acid sequence as defined in (h).

13. A kit comprising of specific antibody molecules or specific binding molecules for diagnosing and/or predicting joint destruction, early joint destruction and/or accelerated joint destruction, wherein said apecific antibody molecule or specific binding molecule is capbel to detect and/or bind to a protein which is encoded by nucleic acid sequence selected from the group consisting of
(a) a nucleic acid sequence encoding an IL-4 receptor (IL-4R) which contains a mutation in position 465 of the nucleotide sequence of the wild-type IL4R as shown in SEQ ID NO: 1, whereby at said position the nucleotide A is replaced;
(b) a nucleic acid sequence encoding an IL-4 receptor (IL-4R) which contains a mutation in position 465 of the nucleotide sequence of the wild-type IL4R as shown in SEQ ID NO: 1, whereby at said position the nucleotide A is replaced by the nucleotide G;
(c) a nucleic acid sequence encoding an IL-4 receptor (IL-4R), said IL-4R comprising at position 75 as shown in SEQ ID NO: 2 a valine;
(d) a nucleic acid sequence encoding an IL-4 receptor (IL-4R), said IL-4R comprising at position 75 as shown in SEQ ID NO: 2 a valine instead of an isoleucine;
(e) a nucleic acid sequence comprising at least 15 nucleotides of the nucleic acid sequence of any one of (a) to (d) and comprising the mutation as defined in any one of (a) to (d);
(f) a nucleic aid sequence comprising a nucleotide sequence as shown in any one of SEQ ID NO: 3 or a fragment of the sequence as shown in SEQ ID NO: 3, said fragment comprising the nucleotide sequence “gtc”/“guc” as shown in position 5758 to 5760 of SEQ ID NO: 3;
(g) a nucleic acid sequence encoding a polypeptide comprising the amino acid sequence as shown in SEQ ID NO: 4;
(h) a nucleic acid sequence which hybridizes to a nucleotide sequence defined in any one of (a) to (g) and having a mutation as defined in any one of (a) to (d); and
(i) a nucleic acid sequence being degenerate as a result of the genetic code to the nucleic acid sequence as defined in (h).


SEQUENCE LISTING (15 sequences)

SEQ ID NO: 1 - DNA, 3678 bp, Homo sapiens
ccccgcgcggcgcgggccagggaagggccacccaggggtcccccacttcccgcttgggcgcccggacggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctctttaaaaaaaaaaa

SEQ ID NO: 2 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 3 - DNA, 26550 bp, Homo sapiens
tacaggtgtgagctactgtgtctggcctgaataataaaatttaaaacaatttttcaaaaattcaccatgaggtctcactatattccctaggctggtctcaaacccctggactccaagtgatccaccccaccttcccgagtagctgggactagagatgcacaccattgcacccaatagagcaatacgtttctgttctttgtaaattacctgctctaaggtatttttgttatagcagcctgtatggactaagctgacttgtaacgttacttgagactttaaagtgttccggtcactgttggagggctctgtctgtgttagctcatttaatccccacaacacctcaatcagatggggctattcttagtcccactttatagataaggaaactgaggcatggaagcacagcttgctcaaggttcacatctagtcagtgacagagcaggtatttaaacctcaggaaataatcagagaaacatgtgtagagggttgtccaaggaaggccacatccagaagcatctcccaggacagttgttgtgtagctcaccctctggactttgtgggtctgggtgttgtttcatgattatagagagagctctgtgaacgtggaggacctgttgtcggcagagacacaaatggccagggcatggctgggcagccgcagtggctcaggcctgtaatcccagcacttcgagaagaccagaggggcagatcatgaggtcagaagttcaagaccagcctggccaacatggtgaaaccccgtctctactaaaaatacaaaaattagccaggtgtggtggtgggcacctgtaatcccagctactcgggaggctgaggcagaagaatcgcttgaacccgggaggtggaggttgcagtgagctgagattgcaccgctgcactccagccttggagacagagcgagactctgtctcggaaaaacaaacaaacaagcaaacaaacaaacaaataaatggccagggcaggggagggttgcatattgaataagatgagctctgctggaagcacaggtcagcactaacctgcttcctctctctctgcaggtgccttggcatctcccaatggggtggctttgctctgggctcctgttccctgtgagctgcctggtcctgctgcaggtggcaagctctggtaagtcaccacttctcaatcattcatttgttggctattaatggcgtgccagggtcctgcagtatgtcacctggccttatggagattacactgcagtgggaggggacagccaatgacaagtggccctgattatcagtaaattctaaagattgttagaaagtgatgggagccgggtgcagtggctcacacctgtaatcccagcacttcaggaggccgaggcaggaggatcgcttgagcccaggagttcgaggtcagcttgggcaacatagggagaccttgtctctacaaataataaaatattagccaggtgtggcagtgcacgcctgtagccccagctactcaggaggccgaggtgggaggatcccttgaactcaggaggtcaaggctgcagtgaactgtgatcgcgccactccactccagcctgcgtgagaaagtgagaccctgtcaaaaaaaaagagaaggtgatggggaaagaacacagaacagcataagagggggttggggaagctgggtggagtgggggggattgcagttgaaagtagggaagtcagggaaggcctcattgagctgacttggaggaagcgggaaccgtgcagatgtctggggaaggctcattcttggcagagaggccctgcactgagcctggcgggagggttgagcacaggagggaatgtggtggaggagagtgagcagcaggagggagcagtgaaggtcagcaaggtgacagagtggctgaatcaaaaaagaccttgcagtgtttgagcagaggatccatatcatccattatgttccaaaggactcttcaggatgccgtgtggagaaaggaagagggtggaagccaggaggtctggagggaggtctggagtggaggagatgagaggctccggatccctctgggaggtagatttgaggacagattggaattgaggtgaaagacagagaaagagaagtggccaggatgactccaagatttctgacctaaactactgggaaggacgcggttgtcatttctgaaatgcagaaggatgccagaagagaaggtactttggggaggggcgggaatcaggagttagttttggacatgagataagcttggaatatttatttgctatctaagacagctccttaacatggtaagcccttatgcaagttgttgtcagctgagatgggcgtggcactgagcatgggagcatggaggcgcctgagtggtctcatgctcaggtggtttagcaaactcagtgtacatcctgccaattccagtcctgccatggccactgacaagctaggagggcgctgaaaggagaaggaccccgatgtctcctccagcccatccatctcctctctcccattggccaaacccaaccggaaactaaaggccaagggtacccggtgatgaagactgtggtatcagcctcctgagcacagagagggcagaaaggggtggagacaaagaggggcgcagatagtgggcaaatggggaagtggcacttcccctagctcgagggcagaggcttggtgtgatggaatggcactccttaaactgctacatattttccctttaatttggccaagaacaagttgtcaagtttgtgtgagataaaggtgcacttggttcgttcttgtctaatggcccccgcacccatgggtatttcttcagcttccacagtcatcccgacactagctgggaagctccagcagccctggtcctggccccagctctgtgggcgctggccctcaactttgcctgcactgtgcttttgtgctattccccttggtcctgtttgggtgcaagtccccctcacgcattgagttcctgggccgctcaggctgctcctgtgtctccccagggaacatgaaggtcttgcaggagcccacctgcgtctccgactacatgagcatctctacttgcgagtggaagatgaatggtcccaccaattgcagcaccgagctccgcctgttgtaccagctggtttttctgctctccgagtaagcctgcgctggagctggaggtttggggaggttgtgcccaaagggtttgccccaagagtgagctgggtccaggtggtgcgctggagtgcaggatgctgagtatggtttgctgctgtttatatggtgttagaggggaggtcccatctccagggacatgttatgtaagatacagtggagcgcatggtgggagtgttggtccacgtggcacatggatacggctggaatactggactagaccagcagttctcacactttttggtctcaggaccctttttcacacttaaaaatgagtgaggacccaaagggctttggtgtaggtaacacatcattctatgtttacctaattagaacttgcaatgaagaaatggtgtaatttttaaaaaattaaaacaattaaaaattttttttcttactgaaatggaggtctcactgtgttgcccaggctgctctcaaactcctgggctccagtgatcctcctgcctccgcctcccaaagtgctgggattacaagcgtgagccgctgtatccggcccaaaatggagaaattttaagtcccaacaacatgcaagcccgcattcaacaaatcttcagatcaattacatgatcacaggtcatgtagcctctagaaaattccactgtacgccagtgagagagagtgaaaaggcaaataacgtccctgtattatgatgaaaagagttttacctggtgggcccagaccacactttgagaaccactggactagacccttgattgaggagtacggtgttgagagtggagtcctctgtgatggtggatggaccaggacacatggcataggagtcaggtggttccctgggctactccatggtgcacaggatgcttcgttacactggtgcccaggacataatcacgtacacaagacacacagttacggggcagactggggatatacggcacaccagcatgcagggttcaccagtaaaggtggtattccatgattattctaaggtagatgggctgtgctttgtttccattggcttagtccagggattggcaaactatggcccgtgagccaaatccggcccactgcttgtttttgtaaataaagttttattggaacacactggctgctgtagttgtaacagaaactgcatggccctcctttatgttttttgtttgtttgtttgtttgtttgtttgttttctttgagacagagtttcgctcttgttgcccaggctggagtgcagtggcacaatctcggctcactgcaacctctgcctcccgggttcaagcgattctcctgtctcagcctcccgagtagttgggattaatggtgcctgccaccacacccggctaatttttcgtatttttagtagagaccggttttcatcatgttggccaagctggtctcgaactcctgaactcaggtgatccacccgcctcagcctcccaaagtgctgggattacaggcatgagccactgagcccggcctcctcctttatcttaattgaaataattcagaaatggaaagtcaaatactgcatgttctcacttataagtaagagttaaataatgtgtacacatgggcattattccatgtaccatggaataacagacattgaagacttgggagggtgggagaggggtgaaggaagagaagttacttaatgggcatagtgtacaccatttgggtgacggacccaccagaaccccagacttcaccactaggcagcatatccagtgagaacagatctgaggcttgccatcaaaattgcacttgtaaggccgggcactgtggtggctcgcggctgtaatcccagccctttgggaggccgaggtgggcagatcacttgaggtcaggagttcgagaccggcctggccaacatggtgaagctccatctctactaaaaatacaacaattaactgggtgtagtggcgcacacctgtaatcccagctactagggaggctgaggcgggagaattgcttgagcccaggaggtggaggttgcagtgagccgagatcacatcactgtactctagcctgggtgacagtgagactttgtctcaggaaaaaaaaacaaaaacaaaaaacaaaaaactcgtaccccctaaatttatacaaataaccaaaaaaaaaaaaaaaaaaggaaattgtgtggctttgaagtccaaaatattaactatctggcctgttacagaaaaagtttgcagacccctggcctagcccgtgagatgtgggttggctgttaaggtggaacattggaattatcttacgatggccaaactgtgcgatgcagagcttatgttgttctaaattaattagtgccaccggttcttccctttcatgggctttcaggaacaagctaagtcccaggaccagggccggcagctaggcaggtgtgaggagcatccttggtgcatgtggtaagaggctgtggccagcaagagaggcaaccctagtcggctgccccagcacaccctggccgctcccaagcccccagatctgtcctcacatccgtgatcgggaagctggaagagtctgatgcggttcctggaggcatgtcccggacacagctgtggggcccagccagcctacaggtgaccagcctaacccagcccctgtgtctgcagagcccacacgtgtgtccctgagaacaacggaggcgcggggtgcgtgtgccacctgctcatggatgacgtggtcagtgcggataactatacactggacctgtgggctgggcagcagctgctgtggaagggctccttcaagcccagcgagcatggtgagcagggcggagtgcggcaggggtggctgggtgtgttcccacagctgcctgggctgagggtggggtgggcaggggaggaggtggggtcatagcaacagcaggaggaagccgcctgtattttcccaaatctgatgggattcctgcccctgcctgggcctcagtcctcccacctttgaaacggagctggtcgcagtagaccaccaagcccccttcagcccagctgtttccacccctgaacttaagtgcccaggaaggcgtattgagatgaggtgtgcttgctggaaggcatgcctgctgctgattgaaaaccgaactgggaacattccttccattctgtgtccactggtcagctgctgcggctttggatggtcttgaccgtggaaggctgaccttcttctggtacccggagtccctgcaggaatcccccttgagcttgctgggctgtggtgacaggagtttaaaacatgcgttgtattccagtgatgcatgatatgacatgcatcacaggaataaaaacctgaggtctcatggatatgattgcttcaaaggagaccaagttttaaaacagatgaatcaaaataaagaaaaatactcagtaaatcatcataaagtacagagatgtggccaaaggtgtgaaggatgcagctgtaaaagctgaagtttgaggccgggtgtggtggttcatgcctataatcccagcactttgggaggccgagcccagcggatcaccggaggtcaggagttcgagaccagcctggacaacatggtaaaaccccgtctctactaaaaatacaaaaaattagtctggcatggtggcaggcgcctgtaatcccagctacttgggaggctgaggtaggagaatggcttgaacccaggagaaggaggttgcagtgagcttagatcatgctactgccctccagcctgggcgacagagtgagattacgtctcaaaaaaataaaaataaataaaaataaaaagattttttaaaaggctgaagtttgggttactttggctcatacactttgccttcactgtagaaaggtggttagtaaagaccaggcgcggtggctcatgcctggaatcccagcactttgggagcccagcgcaggcagatcacttgagccctgggctattgaggctgcagtgagctgggattgtgccactgcactccagcctgggcaacagagtgggaccctgtctcaaaaaagaagaaaaaaagggtaattaataaacactaaagttctatgtagaattttagcaacattattgttattataatcttctttgctatggctctgaatctgtgtggtgctccagaagtatgctatggaggttttgtcgaccaaaaatctgggtggtggctgtggtttgtaggccggggctgggctgggtgatgggggagtcactgcatagatcctcacatagaggccgcttctcccgcagtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatgtgagtgggcatgctttgacgtttttctgtgacctctggggaacagggtgggtgaccagcagaggcccagtccctggagccaggagcctgggaggcaagccctggggctggatagcaaatcccaggagctagagacctggcttctcacctggctctgccctaggcaagtccctttgcttcctggccccccacccctcacatcagagaaggggagttatctctgcatgccgctcctcctctgtaaaggtagggctgtgggccacatctgtgtttcccagtttgggggacacaagtgatcgtaggtggcacattgacagctcacttgaataaccctattattgaagagaataatactgactcaagagacagtgacccgtgtcagttcccttttgaggccaacgggttaaggaggaagtccccatacagctgactcgtttactaattcctcttaatgaagagagcagaggccacaccccaggcttagactttcccaagaaaacaagatcagtttgttggttgttccccatggaagctggtcctgacattcccttcacagtagtgttggtggagtttttgttgttgtttgttttgagacagagtctcactctgtcacccagggtggaacacagtggcgtgatcttggctcactgcaacctccgcctcctgggttctagcgattctcctgcctcagcctcctgagcagccgggactacaggcacctgccaccgtgcccagctaatttttgtatatttagtagagatggggtttcactgcgttggccaggctggtctcaaactcctgacctcagatgatccactcgcctcggcctcccaaagtgctgggattacaggtgtgagccaccgcacctggccagtggagttccttcttaagtacatgtattgacatctttaaaaagggcgagaggatttacaggaaactatcaggtcagtaatggcaggggccgtccacagtgggtggctgagtccccctatttttctgctggtgtgcggggaggtcatttcctgccacccatgtttccccaccctgaatccaccttcctcacattcccattggagggacactctctggacatatgggacctggggtcccacagggctgcattccaatgcctgctgtgccactcgccagctgtgtgatgttgggcatatcccataacctctttgtgcctcagtttcctcatctgtaacacaggagtgacaagagcacccgcccacagggctatgacagtacaaggtgtgtgatacagatgagctcccctgtttggcccacatgtgtcctaaaagccatgtgccctttctcttgagtgccccaggccacagagatccccatctgcccgctgtcccacacactggtctgtcatttgttccttgaggtttgtgagggccggctctgtgcatcccaggggcccaggctgggcctggttggctctcagggagcaggcacccgccaccttaagctcccatgctggtgtctgtcactgcttcctctcaatctggccaagccaggggtgtcgatttatatctctcaggtctggtttcccctttggcactgggccaggtatggggaaagagcaggaatggggcagttggctcacacagcagaggctcagaaagcggggggcatggggggaaggagtgcacagatgctagagagtggggcaagttttgtttggtcaataaatctccttctcatgccccaggcctgtgcaagacctacagagagtcccaaggatgggctggggggaagagaaaggtaccaccttcagagtccaaagatatgttatttaatattttcatatttctagatctgccttcaggcatggctggatccagcttctaggaacctgtccagctctgcgccctgctttattctgtactggcttcgtttttaggcaggctcttccctcatgtagtggcagatatgcctactagttgctccaggcctacatcccaaagccacagtgggaaaagggttttttttcttgacggttctaataagagtcctaaggctgctgctcagtggcctggcttcgatgctgtgccagcctctgaaccaatcactggctgtgggtggagagagggtgctggtggagggccctgcttgtccagggaggagtcacatacctgcctctagggctgcaggtgggctcagctccatccaaaccagatgaactgaaaataaggcaggagtggcttccccaggggaaactggggaagaggaagcaggactgtgctggctaaaatgccagccaggtttaagacgtggcaccagatgccagtcatgggattggattggtcagcatgcctgggctatggcttaggggtatgttggtgctcagggatgccacaggcctccagataccaggtctgaggcagaagaatgaagtccagcttctcttgtgggtggaacagtggcaactgagataccccatctctcccttcccaagaacagagctgaacataaagaatttagtgattggccagagcttggccacatgctcccctctgatgaatgataggccaggtgatgggattggcacaattggcttagactaatgagggttggccctggagttgcaggcagtggagttctgtcctaagcagtgggcacctaaacccgatggcataaaagctgggcgggtgtccacctgcatctgccacagcactgcaggcaccaactgtggctcatactgagtgggataaattccagaaagaaacattaggaacttactatagaattttggggctagagctactcattcattcccctagataatttctaggcaaggttccatagtggagggggagttttggcttgggcattgaaggatgcataggagttttctagatggggaaagaagggaacggtagaccaggcagagggaactgcatgataaaaggtttatgggtgtgaaaattcatggaatgtttgaggattatggggttgggggatgtgggaatatgtgtagcgataaagcaccaaacaaagccaaaagtttagttagagccctgaatgcctgcctcataatggtttccatattttatatgcctactatgtgccaggcacattgctcagggtcacacagctggaaatggcagggctgagtttttgttgttgttgttgttgttgttgagacagagtctcactctatcacccaggctggaatgcaggggcgtgatcatggctcactgcatccttgacttcctgggatcaggtgattctcccacctctgcctcccaggtagctgggactacaggcacaggccaccacgccaggctaattttttgtatttttagtagcgacagggtctcgccatgttgtccgggctggtctggatctcctggcttcaagtgatccccctggctcagcctcccaaggtgctgggattacaggcttgagccaccgcatccagcccagatctgagatttgcacccagtatttgaactcccaagcctgtgctctttttcctcccatggacatttctctcagagatggtctcccaaacacctgtccttcttgttaaaaaacagacaaaccgcaagtagttctttggaagctcagatttctcttttgtttcttagtaaaacatttcccagttcccagctcccttccagggtgtaagatttcttcggtaacttacatctagctgttgcttcttgtttgctcatgtttagaaagaaagacaaaagagagtgagaattttctctcccttccccagtctccccacaactcacaccccaccctcagctccctctgtaataggaaaatctctgaactctctgtagttgctccagcaatcttttggaactttgcttctttcttgtgaaaaaacctccccttggctcactttgcaccaggtttccccaaatgtgcttccaaccacaagcagaaatggagctgccagtaaccaggaagaaactgccgggggctgaggaagaggagagggaggtgcatagccctggatctcgcagggagaggggtgacaggatgagaactcaggttgctcacttgccatcagggtcagtcatgaatatagcgttcatgtatcactttttaaagcttttttggagggtaaaagtaatagttacacaaaataaaaatacaaatggtacaaaaggacttagaatggaaacatgtttctctcccgactccagcctcctgtttttcttcccagagactgaccactgctgtctgtctcttgccagaagggaaagggaggcaaggttagggcaggcagagggcatgtgcatcctttagagagagcttatgtctatacaagcaaatgtgtgtgttcagtcatcgctgtcttagttttctattgctgcataataatggtactaccagcttcacagctttaaacaacacccatttattatctcatagtttctgtggttgggagtctggacatagcttagccaggttctctgctttagagtctcgtgaggctataatcaaggtgtgggatggggctgcagtttcatctgaggctcaattggggaagggtcacttctaagctcatacaatattggtgacattcagtccctggcaggctgttgaactgagagcctcagtttcgtgctggctgttggttgtagttaaccctgaattccttcccatgtgccctttgcaaagccatcaaggcagagagacttgcctagcaagtaggatattacagtcttctgtaatataatcacatccatgaaatcctctatatatcccatcacctttaccatattctgtgggttagaaacaagtagcaggtcctgcccacactcgagaagaccagatgacacaaagatgtgattcaaagtggggatcatcggggccatcttaggtttgtctgcagtgatcactgtgccatctctctctctctctctcttttttttttttttccgagacgaagtcgtcactctgtcacccaggctggagtgcagtggcatgatctcagcttaccacaatctctgcctcccaggttcaaatgattcttctgcctcagcctcctgagtagctgggattacaggtgcccgccaccacacccagctaatttttgtatttttagtagagacagagtttcaccatgttggccaggctggtcttgaactcctcacctcaagtgatccacccacttcggcctcccaaagtgctgggattacaggcatgagccaccatgcccagccccatctctctttaaaaaacaaacaaacaaacaaaaaacataaaaagaagcagagaacacatacacatctgcatcttcccttgtttacttaacaatagatcttggaagtcacttctcagtagaggctaggttgggcagagcattggattctaggccagtgagtttggacttgaccatggagacactaggaagcccatgaaggacagagagagatgcctcgaccctgccagtcctttagaaagatcacccagtgctttttgtataccaaaccctatttgaaatacttacgtatattaacccatttccttatcaccacaaccctgcgggaagggagataggcacttttattatcttcattttgcagatgaggacattgaggtccagagaggttatgtcacttacttaaggtcacacagccaggaagtggtagtagggactcttacccttgttttacagatgagattgaattatctcacgaaaactcagaaaggttaaacaacttgcctaagtaacatacagctaattagtcgaggagcctgacgcatgttgctctagcctggtcacagttacagaggtggcaagcaatggcctgaacaggacgaacaaccaaatacccaggctggtggctcttaaacatggtggggtcagctaacgacagcaaccagggtgggcactggtgcccctcgcccccggctggtgccctaacatctcccttttctctaccagttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactgtgagtatcaagaggcctaagcaatggtaatctccactctccattcttcccctgtggccagacacttcccctggctgagtctctgggcttttatatcataggatgcctctaatggcaatcctgccattagatacacctgccgtggtgtatctgccaggtaggcaggctaggctgcagtaacacacaagcccacaatttccatggcttaacactataggaatatatttcttgctcacgtaacaagctaacgtgaatgttgctggtttgtaggtggtttccctccctgtagaaatctggggagtgaggttctttccatcttgtggtgccatcattctccaggacaaagattcttacctacttttgtgtcctggtttcctttggcagcctggtgaagcctatggacctcatttcagaatatttttaaatacataaaatcccagcctgggcaatatagtgaaacccccatctgtacaaaaattagccaggcatggtggcatgcacctgtagtcccaggtactgggaaggctgaggtgggaggatcacttgagcccaggagtttgaggctgcagtgagccgtgatcgtaccactttactcccacctgggtgacagagcaagagcccatctctaaaaataaataaatacaatgaaataaaataaaataaaataaatagaactacagaggaaactaattgtattgaaatgcagttataaaacatttaaacacatttttaatctagagatatatgtgcttctttattaagatctataaataataagttctaggggtagctcgcataaatactgtaatttcaaagtagataagcataaataatactttatgatactgaaattgtgatgtgatatgagaatagctgtgagttttgttttgctggggacaggatcactgatgctgtcattactggggtctcttccctccattctttttttaaaattgtattttattttatttttaaaattttaaaataaatagagacagggtatcactatgttgcccaggctgcttttgacctcctgggctccagtgatcttcccatcttggcttcccaaagtgctgggattacaagtgggagccagtgttcctggccccttcctccattcttaatggaaggagatgctaggtgtgagaggttagggaaagtaaagatgtaatttctttcccatccaagttctcagacccctgaattctacctgcagccatgttggtccatcaaccccaagtgaagaatccctgctctagggccccaccattgtctgtatccagccagcagaagaggcgtgattatggagatcacatctgcttcttgaaagcagacagcccggaagtgggccgcatcacttcctctcaaattctattggtgaaaatggtcacatgactacacatagccacaaaggaggctgggaactttctcacttggaacctacatcccagaaacaactcttttcagtgaggtatcccacaggtctttcgcagtagaaatattgattatctcacataaaatgaagtcttacaaatggacctactgggttttgtacagcagccaagtgatatctcttcccttctgctgtcttcccttctgccatccttcacatggtggcattgtatccttagacttgccacccatgccctcaggttggccgttgcacactgtcttacataaagcaggaaggaaaggaaaggctgctacgagagagtgtaccttgtgcatctcttttttaatcaggaagcaaacatctttctagaagcttccctagcaaaattccccttacatctcattggccaagactgttacatgttacatggttactgttattacttgctcattgcaaggaagactgggaactcaaatgcctggaaaaaggaacaggataatcgtgattggctcaagccttagggtgggcatggctccctgacaagggagagaggaaaaagctgttgagtgaagaagactgcttcagtttccccatctgtataatgggaggagtaagggctgtcgtgaaaactcaatgaaagaagattcttcaacgtggtaggtgcagtggcagctggcagtaccctgaccctgccaccgcacagccctctcagcattgctcatcctgcactgtggatatcagttgagccacgtgtctcctgccctgggctgtgagctccataggcagggtctccatggctgtatctccagaacccagcacagaaccaggtgcttgggaaagttttgaattgattctcatctgccattggcatggggaagggaactagcttgtatgaaacagataacaatgtatgggaccctcattcattatttcagcaaatatttgctgagttcctcctacatggctagccctgtgctagacactggggaatcggcgatgaacaaagcagatagaaatccccactcttgtggagctgacattctggagggagagacaaaaagcaaacatataaagaaagaaagaaatcacatggatctggatgacagtgagtgctgggaagaaaataaaagcagaggaaggggatggagcgatgggcagggggcaacggtagggagggtgtcggggaaaactttttggagaatgtgacgatgaaagtgaacaaggagaagtcaaccgtgttgagatgatggcagctaatgatgtggacaggccactctgttctgagtgcattatctattgattcatcatgtcatcctcgcaacagccctgcacgatcaattctgtcattaaccccatagtacagatgaggatgcggaggcacagagaagataagggacttgtcctgtgtcacacagcaaggagccatccggctcctaagttggtgcatttgacttctgtgcttccggaaagaaagagcagcaagtttaagatctggaggtggcactgagctttggaggagcagggggcaatgaggtggccggtgtgacgaggactcaatgtgcaagagggagagtggtggggagatgaggtggaggggtggtcggcggtcagatcgtggagggtctcggacgagggtcctgaccctgggtctccagtcctgggaagtggagcccaggctgtaccatggctgacctcagctcatggcttcccctcccacttccagcctacagggagcccttcgagcagcacctcctgctgggcgtcagcgtttcctgcattgtcatcctggccgtctgcctgttgtgctatgtcagcatcaccaagtgagtcctgggcccagtgctgccgagcagtccctctggagtgcagggtggcagggacttgcccctctagtctgcccctttgcagtcctctcagtcaataatacgtatttactgagcagctactacacaccttgagagtagagctgagaacatatcgacaaggaccccacttttttcttttttttttttttttttttttttgagacggagtctcactctgtcacccaggctggagtatagtggcacaatcttgcctaacagtaacctccgcctcccgggttcaagcaattcttctgcctcagcctccagagtagctgggattacaggcgcatgccactatgcccggctaattttttgtatttttggtagagatggggtttcaccatgttggtcaggctggtctcgaactcctgacctcatgatctgcctgcctcagcctcccaaagtgctgggattacaggtgtgagccactgcacccaaccaggactccacatttctaaaaccggcatcctactggggagactgaaaatacatatcaatcacaaacaggtggttttccatagtgacccactctctgaatgcactagaccagggtggaggccagagatcttctggggtgctttttgcaagggggaccaggataaggctctccaaggagggaaaatttgaggggggccctgactggggagaatgagctggccagggataagcaagatggagtcatcccacatccccttacaacactgggtgcctgggcaactgggggcatttgggggcatgtggtaggagccagaggaatttgcgacgattgccctgatggagtcaggagacctgggtttgaatcctggccttggagcttggtagctggcggccgacaagttgctgaaacccctgagcctggggttcctgctttgcagagtgacagtgatggtgagaacatatttcatcagccagaagaggccaaatcacagtaaaggctgagggaggagatgagtggcgagtggctgggaggtggtggaaggagcctcgtttccagagagctcttgccagcccttggaatcatggtgtctcagagcctcagtcctcccatctctgaaatgggactagcaagctcaacctcactaagtcaggattagaggtggctaaggattattaacatgattgatgaaagtgcccactcttggcccagcacacactaggtaggcagggaatgcaaattcccctccatatcttgtcactgatgcctccgagcaaccttggactgatcgccttgctctgagcctcagtttccccatcacctgtacctcttcccactccccatcactatatcccagcatgccagcctctttgctgttctttgtctttggtttcttgttttgttctgttttttagacagggtctcactctgttagccaggctgaagtgcagtggcgcggttacggctcactgcagcctccaattcctgggctaaagagatcctcccatttcaacttccagagcagctgggacaacaggcgcttgccaccacacctggctaattttcttattttaatttaattttattttattttttgggacagagtggagtctcaaaaaccaagctagagtgcagtggtgcgatctcgactcactgcaatctctgcctcccgggttcaagcgattctcctgccttagcctcccgactagctgggattacaggcgtgtgccacgacacccagctaatttttgtatttttagtagagatggggtttcaccatgttggccaggatggtcttgaactcctgacctcaagtgatccacccacctcgttctcccaaggtgctgggtacaggcatgagccactgtgcctggccaattttcttacattttgtagagactggctgtcacttatgtagcccaggctgatcttgaacttctacccctttatctttattcatggcacttattaccatgaatgaatgacctcatataagcatttctttcgtttttttttttttttctttgagatggagtctcatgttgtcccccaggctggagtgcagtggcgcgatctcagctcactgcaacctccgccttccgggttcaagcgattctcctgcctcagcctcctgagtagctgggattgcaggcgcctgccaccatgcctggctaagttttgcatttttagtagagacggtgtttcaccatattggccaggctggtctcgaacttctgacctcaggtgatacacctgccttggcctcccaaagtgctgggattacaggcgtgagccgccatgcctggcctcatataagcatttctgtctccatttatcatccatctttccctcttgaaggtcagtttcaccaaggcaggcatctttgtctcgttcactgttgtggcctcagggccaggcacagtgagtcaaacatagaaggtgctcaataaatatgtgtttatttattgaaaccatgggcagaggctaattcagaagcggtctgaggaccttacctcccagtgatgatgcaccatggccccaggcaggccaggaagagagaagggttgtgtttctccgtaggtcccccagcttcccaggccatcccaggccattccctggtcatttgccctcagctgctctgaaaaagggattgttgaggggaacctagaatcctctctctgcagtttgagtctttcctaatcccctggggtctcattcccactgaggacataggtggcctcctcaggaactctgtgctgggtaacagaatgcgggagtgtgaacctggctctgccacctaccagctgtcactccacctccttgggcctcactctcctcatctgtagaatagggttagcaatagaatccatgtcaccaggttagaatgatgagtcagtggtttgacctccagaaactaatcagcctgatctctgatgccaaataagtattggtgataacgaccacttttatgggaggagcgttcacctgtcaataattcagagatcaacaccttttccttttgtttttcaggattaagaaagaatggtgggatcagattcccaacccagcccgcagccgcctcgtggctataataatccaggatgctcaggtaggagtaggcgtggatgaggacatgtgggactgtgtacatgaagaagtgtggttcagaacacctgggctgttaaggaccttcactggcttctggaatggcaaatagacagtcaggagggttgcaggggagacagaggcagaagccgaatgaggtcattagcagaccagaggctttcccgcccttccccttggcaatcccagcctggggtgggcttctctggggttggtttcctgtttttttccctccccttgggagaatgacccttgggtcatcatcactgtgtcattccctggggaggtgccagtaccagggctagaggccagaaggagtggaggaaggagagggtgacaggctttctgtgtcttcttcttaagcataggaaactgcccccgaagcactagcaaatcccttccgggttctcattggcctgaaatgtatcccacccctaagccaggggtggagtcagcttccccaaggcgatggtcctgtgggtgagtgggtggggtttgcctgagcaagatgagagttctctaggtaggagaaagggggattataggtcctgtctagaagagaaggtctgagggtccttgcttttccagggactctggaatctagtggtgttggctttgaatcctgactctgccactcactggcagtgtggacttgagcaagttgcttaattctctgagcctcagtttcctcttgtgggttataacagtgtttacctggtaggacagatattggaatttattgagacaatacatataaagtgcatattccagcctcttgcaaataccaagtgccatttatgtatcagttagtgtttgctgtgtaacaaatgaccccgaaatgtagagggttacaacaactttatttagcttatgcttctgcaggctggcatttggggctgggctcagcagtgagggtggcgggggaggctgggctgggctgggctgggcagatctgaattgagctgacccgtccccgtagcctccctccgtgtctgacagttggctttttttttttttttctttttctgagacggagttttgctcttattgcccaggagtgcaatggcgtgatcttggctcactgcaacctctgcctcctgggttcaagcaattttcttgcctcagcctcccaagtagctgggattacaggcatgtgccaccacgccaggctaattttgtatttttaatagagatggggtttcttcatgttggtcaggctggtctggaactcctaatatcagatgatccacccacctcagcctcccaaagtgctgggattacaggcgtgagccactgcacccagcctagttggctgacttttacctgggacagtgcaggtgcctgagccatgtgcctctcactctccagcaggccggcccaggcttgtttacagagtggctcagttttcaagggtgggaagtcccaaggcttcttgaggcctaggcgcagcactggcatgatatcacttccatcacattctatgggcccaagcaagtcccagggccagtgtagattcaagggatgggaggagattcagagcactcctctgtggccacttttgccatcgaccacagtccctgtaaatattaggacaatgtaattaattcccaggaatctgaggctcagaaagcgtaagtgacctgttggacttctgatctgtgtgatgtcgaggcttgtaccccttcctgagcattgccgtactccaggccgggctgcaaggccactctgctctttcattggctgtctctgtattttaggggtcacagtgggagaagcggtcccgaggccaggaaccagccaagtgcccgtatgtatctgaacttaggtcacagcctgcatgcattgggaaggtgatagaattggagaggcaagcccctagctccatgtctgccttctcttccctgcattcggtaattgccctgtgacattagccttcaagggacggcaggaggaggggtgttctggaaacgtggactgctggccaagccccctgagtttcactggtgtgtcaggtacatggtgataccccttgggagtgctgttatagttaacaaccagagcagccgtgcctgttgttaaaatcttgacctaattgtatacttgtcggcaaatagccactatcctgaacactcccctccttttttttaatatacaggatctcactctgtggcccaggctggtgtgcagtggtgcgatcatagctcactgcaccttcaaactcctgagctcaagtgatcctcccatcttagcctcccgagtagctgatactacagatgtgcattaccacgcctggctattttaaaaggtttttgcctgtaattccagctactcaggaggctgaggcatgagaatcacttgaacccgggaggcagaggttgcagtgagcgcagattgtgccactgcactccagcctgggcgacagagtgagactcttgtctcaaaaaaaataataccaaaaaaagtttttgtaaagacaagctctcgctgtgttgccccgccactgtggcctccttagcttcttccctggggcctgctggacctttccatactccagaaactaaagggggtccaggaccctgcttcaaccctaggatcccgcatcttttttttttttttttttttggacgcagggtcttgctgtgtccctcaggctggagtgcagtgattcactgcagcctcaaactcgtgggctcaagtgattctctagcctcagccttctaagtagctgggactacagtcatacaccaacatgcccagctaattttccttttttttaattcttgtagagatgtttgagacggcttgggctctgttgcccaggctgttctcaaactcctgagctcaagcgatcctccctcctcagcctcctaaagtgctgggattacaggcgtgagccaccgcacccggcttccatatcctttctaattggtcatggcttgggataatggtgttgcttttaattatcatcatccataaagactttttcttactcaacagatctgagcttgtatttggtgcccaggacatgtgctgggttcccgaaatcccaaagacacagaccctaccctcagggatttctcattctagcaacatagactgatcaattactgattataacgttagaaggcatgtctgaagtagacagccatcaggacatggtgatttcaggctgggctttgaagaatgaataggagtttttcaagtgtcgaaactgaaccctgaccaacctttgcttttgcagacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctctttattttttatgtattaaccaaacatacctccagacactgctgtgagtgctgtgtctctgttaactcctggaattcacccatccagaggaaccaggatgcaagaggttaagaaacttgccatctgggtttgggttccccatacaaggattcaaatagttgatttaggaagtaatcccgggaaaccctgctaaggtagtggggaactgaggcagggaaggacacaaaccaagaaagtgttacctgaaaggggtccagatgcagaccccaaaagagggttcttgaatctcatgcaagaaagaattcagagcgagtccatagagtcagtgaaagcaagttaatgaggaaagtaaaggaataaaagaatggctactccgtagacagagcagccctgagggttgctggctgcctatttttatggttattgattaattatattccaaacaaggggtggattattatgcctcccttttagaccatatagggtaacttcctgatgttgccatggcatttgtaaactgtcatggcgctgttgggagtgtagcagtgaggacaaccagaggtcactcttgttgccatcttggttttggtgggttagagccatcttctttactgcaacctgttttatcagcaaggtctttatgacttgtatcggtgacgacctcctgtctcattctatgactaagaatgccctaacctcccaggaatgcagcccagtaagtctcagcctcattttacccagcccctcttcaaagctccagtttaaataaacctctgacaaaagggtgagttattcaacagattaccagcatgagcaactgatgcttacctgccggggatctctggaagaccatgcatggcacatgcccagttatgcctgcaaaggagagggagctg

SEQ ID NO: 4 - protein, 825 aa, Homo sapiens [sequence omitted]

SEQ ID NO: 5 - DNA, 15 bp, artificial sequence
  primer
acacgtgtatccctg

SEQ ID NO: 6 - DNA, 14 bp, artificial sequence
  Primer
cacgtgtgtccctg

SEQ ID NO: 7 - DNA, 17 bp, artificial sequence
  Primer
acccagcccctgtgtct

SEQ ID NO: 8 - DNA, 16 bp, artificial sequence
  Primer
cgcgcctccgttgttc

SEQ ID NO: 9 - DNA, 20 bp, artificial sequence
  Primer
gccgaaatgtcctccagcat

SEQ ID NO: 10 - DNA, 19 bp, artificial sequence
  Primer
tgctccaccgcatgtacaa

SEQ ID NO: 11 - DNA, 17 bp, artificial sequence
  Primer
cagtggctatcaggagt

SEQ ID NO: 12 - DNA, 17 bp, artificial sequence
  Primer
cagtggctatcgggagt

SEQ ID NO: 13 - DNA, 24 bp, artificial sequence
  Primer
tccagatccagcaaatagcacttg

SEQ ID NO: 14 - DNA, 22 bp, artificial sequence
  Primer
agtctatcaggagcctgtccaa

SEQ ID NO: 15 - DNA, 18 bp, artificial sequence
  Primer
ttgcagaggagaagacac